(19) United States
(12) Patent Application Publication
(10) Pub. No.: US 2018/0264464 A1
(43) Pub. Date: Sep. 20, 2018

(54) NITROCELLULOSE EXTRUSION FOR POROUS FILM STRIPS

(71) Applicant: Grace Bio-Labs, Inc., Bend, OR (US)

(72) Inventors: Charles Greef, Bend, OR (US); Joshua Snider, Aloha, OR (US); Steven Weaver, Bend, OR (US); Jennipher Lyn Grudzien, Bend, OR (US); Kelsey Anne Muller, Bend, OR (US); Stephen E. Moody, Woodinville, WA (US)

(21) Appl. No.: 15/989,045

(22) Filed: May 24, 2018

Related U.S. Application Data
(63) Continuation-in-part of application No. 14/931,512, filed on Nov. 3, 2015, now Pat. No. 9,981,264.
(60) Provisional application No. 62/075,126, filed on Nov. 4, 2014.

(51) Int. Cl. B01L 3/00; G01N 33/487; G01N 21/64

(52) U.S. Cl. CPC B01L 3/5023; G01N 33/4875; B01L 3/502715; G01N 21/6428; B01L 2300/0825; B01L 2300/025; G01N 2021/0112; B01L 2300/0636; B01L 2300/0645; B01L 2300/069; B01L 2300/088; B01L 2300/16; B01L 2300/12; B01L 2300/0609

(57) ABSTRACT

Methods and systems are provided making a porous nitrocellulose strip for use in a lateral flow test. The strip may be formed from a liquid polymer mixture dispensed onto the planar surface via a dispensing device positioned vertically above the planar surface. The strip may be printed with a microarray of binding ligands for use in the lateral flow test. Methods and systems are further provided for developing and viewing the results of the assay.

### CROSS REFERENCE TO RELATED APPLICATION

The present application is a continuation-in-part of U.S. patent application Ser. No. 14/931,512, entitled “NITROCELLULOSE EXTRUSION FOR POROUS FILM STRIPS,” and filed on Nov. 3, 2015. U.S. Non-Provisional application Ser. No. 14/931,512 claims priority to U.S. Provisional Patent Application No. 62/075,126, entitled “NITROCELLULOSE EXTRUSION FOR POROUS FILM STRIPS,” and filed on Nov. 4, 2014. The entire contents of each of the above-listed applications are hereby incorporated by reference for all purposes.

### FIELD

The present application generally relates to methods and systems for making nitrocellulose polymer films, methods and systems for casting those films, and the use of those films in the qualitative and quantitative analysis of biomolecules in a sample.

### BACKGROUND AND SUMMARY

Lateral flow assays (LFA) use a porous polymeric film, usually comprising nitrocellulose (cellulose nitrate) on a carrier substrate, to provide a wicking medium to transfer liquid that contains assay components from an origin through a region of immobilized ligands, wherein interaction of binding pairs and detection of bound ligand pairs can occur.

LFAs are commonly used as diagnostic test devices to detect the presence of biological molecules in a sample. They generally use capillary action to flow solutions containing biomolecules through a porous strip. As the sample passes through the strip's pores and regions, analytes bind to the porous polymeric film. LFAs may also contain binding ligands specific to an analyte of interest, which assist in the capture of any molecules of the analyte of interest. Such binding ligands can be used to detect biomolecules such as antibodies, antigens, oligonucleotides, and RNA for a variety of clinical and research purposes. Labeling methods that allow visualization of the bound biomolecule complex can then provide determination of the presence and/or absence of the biomolecule of interest.

One example of a LFA test is the human pregnancy test. Other common applications are related to the detection of toxic compounds, infectious diseases, allergens, chemical contaminants and illicit drugs, etc. LFA tests are particularly useful in the area of point-of-care testing, eliminating the need for time-consuming laboratory work and providing visual test results within a relatively short time frame, such as in 5-30 minutes. LFA tests are also used in academic and research settings.

Methods of making such lateral flow assays devices as described above are described in WO00/08466 by Freitag et al. (U.S. Pat. No. 6,214,629 B1). Described therein is a diagnostic device that incorporates both a dry porous carrier in the form of a nitrocellulose sheet, and a housing for that carrier that incorporates a sample inlet.

However, the inventors herein have recognized potential issues with such systems. As one example, the LFA devices by Freitag et al. and others are cumbersome and labor intensive to produce because of the cutting and assembly steps required to fabricate the final device. LFA devices are typically constructed in a multi-step process in which the nitrocellulose film is cast to a large sheet, functionalized with immobilized capture ligands, blocked against further protein binding, cut into strips, and assembled into a single use device. The process is time consuming and contributes a large fraction of the production cost as well as introducing variability between tests.

Further, the detection methods of current LFA devices are relatively crude. Many LFAs, such as a pregnancy test, are created to identify a single antibody, antigen, oligonucleotide, or RNA. Current LFA diagnostic tests are available for a limited number of pathogens, and separate LFAs are frequently required to identify each pathogen of interest. Further, LFAs are generally designed to show a positive or negative result, which may not provide sufficient information for a diagnosis to be made. LFAs may also have significant variability in sensitivity and specificity compared with gold-standard diagnostic techniques, such as bacterial and viral culture techniques.

Tests with higher sensitivity and specificity than LFAs include nucleic acid amplification tests (NAATs). However, while NAATs may enable some multiplexing to test for multiple pathogens, their capacity is limited. Furthermore, NAATs are expensive and require extensive infrastructure, limiting their usefulness in resource-limited locations. Additionally, NAAT results may require significant interpretation from a highly trained technician, unlike the simple positive or negative result of LFAs.

Conversely, lateral flow microarrays are useful tools for highly multiplexed determination of the presence or levels of clinically relevant biomarkers in biological samples. The highly multiplexed microarrays may allow for the fast and inexpensive detection of multiple causative agents of a disease or condition from a single patient sample. Furthermore, the ability to use such microarrays in remote, resource-limited testing locations would enable not only a direct medical diagnosis, but also surveillance and public health monitoring. However, such microarrays have had limited utility in disease diagnosis due to the low sensitivity of existing systems.

The issues described above may be addressed in a variety of ways as will be described in further detail herein, including methods and systems for manufacturing polymer strips, the use of such strips in the capture of analytes of interest present in a sample, methods and systems for the quantitative and qualitative analysis of the captured analytes of interest, and methods and systems for analyzing the results of such an analysis for the use in the diagnosis and treatment of various diseases and conditions.

In one example, the issues described above may be addressed by a lateral flow assay device, wherein the device is made by casting a polymer mixture containing nitrocellulose directly to a substrate or device housing. This direct casting method eliminates multiple processing and assembly steps. In other aspects, this direct casting method allows for the modular construction of lateral flow microarrays with single or sets of microarrays on nitrocellulose strips. In one aspect, one or more combinations and formulations of the components of a polymer mixture, including, but not limited to a solvent, non-solvent, and nitrocellulose, as well as the conditions under which the mixture is allowed to polymerize and dry, may be regulated and altered to achieve a desired pore size and uniformity of a porous nitrocellulose strip. For example, the relative humidity and/or the temperature of the environment in which the nitrocellulose strip is cast and cured may be adjusted to regulate the rate at which volatile components of a polymer mixture evaporate. By adjusting the rate at which the volatile components evaporate, the resulting pore size of the nitrocellulose strip may be adjusted to a desired pore size. In this way, the resulting strip has wicking and biomolecular binding properties that allow development of desired lateral flow biomolecular detection assays. In some aspects, the drying and polymerization process may be manipulated so that different sections of the nitrocellulose strip may have different pore sizes, either in distinct sections, or as a gradient from one end to the other, allowing for increased complexity in biomarker analysis as different regions of the nitrocellulose strip may be used to capture differently sized biomolecules.

The polymeric strip may be used by itself, or may be chemically sensitized to encourage binding of analytes of interest. For example, it may be impregnated with binding ligands that bind to the analyte of interest. Such binding ligands may be attached to the polymeric strip in any way generally used. In some examples, the binding ligands may be printed on the polymeric strip, for example as a microarray. They may be printed using any means generally used including, but not limited to, with fine-pointed pins, using photolithography with dynamic micromirror devices, ink-jet printing, electrochemistry and the like.

The polymeric strip may additionally include positive and/or negative control spots or binding regions separately or as part of a microarray of binding ligands for the analytes of interest. An analyte of interest may be bound by only one type of binding agent or a plurality of binding agents, for example an analyte of interest may be bound to both an immobilizing agent and a labeling agent. In some examples, the affinity of binding ligands to the analytes of interest may be manipulated by altering a variety of conditions, including, but not limited to pH, time, temperature, and buffer composition.

In some examples, a method of creating polymeric strips may comprise positioning a dispensing device a threshold vertical distance above a substrate, dispensing a liquid polymer mixture from the dispensing device onto a planar surface of the substrate, and while dispensing the polymer mixture, moving the dispensing from a first position to a second position. Further, the method may comprise, in response to the dispensing device reaching the second position, terminating the dispensing, and drying the mixture.

Another aspect includes a method for producing a nitrocellulose strip on a substrate by using a dispensing device; providing a removable framed mask on top of the substrate to define the shape, size and thickness of the strip; dispensing a nitrocellulose-based polymer mixture through the frame onto the substrate; and spreading the dispensed mixture with the dispensing head in a programmed fashion.

The polymer strips may be applied to any type of substrate generally used. In this way, separate sheets of nitrocellulose may be avoided, and thus improved manufacturing may be achieved. An advantage is the ability to produce nitrocellulose-based strips for LFA comprising a plurality of pores of a uniform size due to controlled evaporation of the components of the polymer mixture without the need for inefficient processing and assembly steps. This enables an automatable fabrication process that will result in more reproducible products than those currently available with multi-component devices assembled in a multi-step process.

In another example, a lateral flow device may comprise a housing comprising an upper first portion and a housing base. The upper first portion may contain a sample port and one or more windows capable of providing viewing access to all or part of the contents of the housing. For example, enclosed within the housing may be a substrate including a planar surface with a nitrocellulose strip (also referred to as a matrix strip) disposed on the planar surface. While the nitrocellulose strip may be created by any means generally used, in some examples, it is created and applied directly to the substrate as described above. The nitrocellulose strip may comprise one or more analyte capture zones comprising one or more binding ligands. The binding ligands may be placed in any arrangement useful for the intended purpose of the invention. In some embodiments, one or more microarrays of binding ligands may be printed on the nitrocellulose strip. The strip may be of various forms, including linear, curved, S-shaped, sinuous, and/or angled. In some examples, the substrate with the nitrocellulose strip may be removable or replaceable within the housing. In other embodiments, the housing may be sealed. The one or more viewing regions in the upper first portion of the housing may provide physical and/or viewing access to the analyte capture zones and/or an identification region which may provide information about the manufacture, analyte capture zones, and/or sample identification in computer readable or visually readable formats, or both.

The lateral flow device may additionally comprise one or more reagents and one or more fluidic gaskets constructed to guide the one or more reagents to a reaction or incubation chamber. The reagents may be in one or more sealed liquid reagent packs which may be coupled to one or more of the upper first portion of the housing, the substrate, the fluidic gasket, the housing base or a combination thereof. Such sealed reagent packs may be actuated by any means generally used. In some embodiments, they are manually actuated through the housing base. In some examples, the lower second portion may further include a waste collection material to absorb excess liquids from the device.

The use of the nitrocellulose-based strips produced with a plurality of pores of a uniform size or sizes without the need for inefficient processing and assembly steps to carry microarrays of binding ligands allows for the creation of reproducible products that can be used with an image acquisition system with high sensitivity and rapid diagnosis. In some aspects, the captured biomolecules may be tagged with a fluorescent label. While any fluorescent label may be used, in some aspects the fluorescent labels may comprise organic fluorophores (e.g., dyes). By using organic fluorophores to label the biomolecules, field stability of the fluorescent label may be increased while costs are decreased and the sensitivity and resolution of the images captured by the image acquisition system are maintained, allowing for improved accuracy in the diagnosis of infectious disease.

In one example, the imaging issues described above may be addressed by an optical assembly that may be user alone or as part of a larger microarray assembly. The optical assembly may comprise one or more light sources such as lasers configured to illuminate ligands bound to a substrate. In some examples, the ligands may be fluorescently or colorimetrically labeled. The light sources may emit the same or different wavelengths of light and may be placed parallel to one another and perpendicular to the axis of the substrate to minimize the size needed for the device. While the light sources may be positioned parallel to one another, the light emitted from the one or more light sources may be angled to engage with the fluorescently labeled ligands bound to the substrate with or without the use of mirrors. The optical assembly may additionally include a camera positioned to capture light emitted from the fluorescently labeled ligands bound to the substrate. The camera may be positioned parallel to the light sources and centrally located between two or more light sources such that the camera faces the portion of the substrate comprising bound ligands. In some examples, a plurality of switchable detection filters may be positioned between the camera and the labeled bound ligands. Such filters may be positioned to be mechanically manipulated in any way generally used, and in some embodiments, they may be manipulated via controlled linear motion, allowing different filters to be placed in front of the camera, between the camera and the labeled bound ligands. Generally, the light sources may be arranged off of a common axis of the camera and the switchable detection filters. In some examples, the camera may be moveable along an axis parallel to the substrate and perpendicular to the common axis, allowing the camera to be repositioned in front of different sections of the substrate, for example if the substrate has multiple regions of labeled bound ligands. In some examples, the optical assembly may be encased in a housing including an antireflective window position between the switchable filters and the substrate. In other examples, the antireflective window may merely be placed between the filters and the sample. The light sources of the optical system may be used singly, jointly, or in any combination thereof.

The optical system may be part of a microarray assembly including at least one display, such as a tablet, monitor, desktop computer, touchscreen, or mobile device. In some examples, the microarray assembly may comprise one or more compartments which house one or more elements of the microarray assembly. For example, the optical system may be in a first compartment and a controller and/or other electronic boards and components may be in a second compartment. The user interface, power source, and/or additional peripheral devices may be housed in additional compartments or may be separate. The user interface on the display may allow an operator to manipulate the light sources, including the wavelength and frequency of the light sources, detection filters, and positioning of the samples as well as provide data analysis and clinical results classification. In some examples, multiple images of the same assay under different excitation or filter conditions may be captured, facilitating the qualitative and quantitative analysis of different biomarkers on the same nitrocellulose strip. The user interface may be connected to the microarray system in a wired or wireless manner to securely store and/or transmit the collected data. In some examples, the collected data is transmitted directly to the cloud, allowing for remote access or analysis, for example, in instances where data is collected in the field. Such a system may be packaged with suitable reagents, allowing for the detection of biomarkers and the diagnosis of a plurality of diseases without requiring a laboratory setting. The microarray system may further include a sample insertion pocket configured to receive a substrate with a porous nitrocellulose strip printed with a microarray of binding ligands, wherein the porous nitrocellulose strip has been contacted with a biological sample and labeled with molecular probes. Such molecular probes may be excited by the light sources of the microarray system, emitting light to be capture by the camera and qualitatively and quantitatively analyzed by either the microarray assembly or a remote device.

It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF DRAWINGS

FIG. 1 is a schematic depicting a lateral flow assay device and steps associated with production of an individual lateral flow assay strip.

FIG. 2 is a schematic depicting another embodiment of the lateral flow assay device of FIG. 1, including a mask, and steps associated with production of an individual lateral flow assay strip.

FIG. 3 is a schematic depicting the lateral flow assay strip of FIGS. 1 and 2, disposed within a chamber.

FIG. 4 is an example method for production of a lateral flow assay strip.

FIG. 5 is an enlarged view of an example of a substrate in the form of a chip.

FIG. 6 is an enlarged view of examples of a lateral flow assay strip.

FIG. 7 is an enlarged view of an example of a substrate and housing for a lateral flow assay.

FIG. 8A is an exploded view of a substrate included in a cartridge.

FIG. 8B is a top view of an embodiment of the cartridge of FIG. 8A.

FIG. 8C is a top view of an alternate embodiment of the cartridge of FIG. 8A.

FIG. 8D is a view of an example of a fluidic gasket that may form a component of the cartridge of FIG. 8A.

FIG. 9 is an example method for printing a microarray onto a nitrocellulose strip.

FIG. 10 shows a schematic of a first example of a biomolecule analysis system for identifying and/or quantifying target biomolecules, the analysis system including a biomolecule microarray assembly.

FIG. 11 shows a schematic of a second example of a biomolecule analysis system for identifying and/or quantifying target biomolecules, the analysis system including a biomolecule microarray assembly.

FIG. 12 shows an example of an optical assembly that may be included in a biomolecule microarray assembly.

FIG. 13 shows an exploded view of the second example of the biomolecule analysis system.

FIGS. 14A-14C show external views of the second example of the biomolecule analysis system.

FIG. 15 shows a block diagram of example electronics that may be included in a biomolecule microarray assembly.

FIG. 16 is an example method for imaging a microarray using a biomolecule analysis system.

Note that the drawings are not to scale, and that as such, other relative dimensions may be used. Further, the drawings may depict components directly or indirectly touching one another and in contact with one another and/or adjacent to one another, although such positional relationships may be modified, if desired. Further, the drawings may show components spaced away from one another without intervening components therebetween, although such relationships again could be modified, if desired.

### DETAILED DESCRIPTION

The present application relates to a lateral flow test device comprising a porous nitrocellulose-based strip and a method of producing the aforementioned strip by utilizing a dispensing device programmed to spread a polymer mixture into a pre-defined shape, and methods of using the porous nitrocellulose-based strip in the detection and quantification of biomolecules such as antibodies, antigens, oligonucleotides, and RNA for a variety of clinical and research purposes. The polymer mixture includes at a minimum a combination of a solvent, a non-solvent, and nitrocellulose. Upon drying, the polymer mixture becomes a nitrocellulose-based strip. In one embodiment, the solvent may dissolve the nitrocellulose, while the non-solvent may be miscible with the solvent at a given concentration, but may phase separate when the non-solvent concentration exceeds a certain threshold.

In addition, the application provides various formulations of a polymer mixture, wherein the polymer mixture may comprise variable proportions of one or more solvents, meta-solvents, non-solvents, and/or nitrocellulose, additives, etc. Furthermore, conditions under which the polymer mixture may dry to yield desired characteristics, such as a particular pore size, are provided. Capillary flow of liquid through a nitrocellulose film is, in part, dependent on the pore size of the polymer film; therefore, by controlling the pore size of the polymer film, one can control the flow rate of liquid through the film. Achieving a desired pore size may also enable maximal detection of a particular protein in a given assay. Such manipulation of pore size may be contingent on a selected combination of solvent, non-solvent, and nitrocellulose, as well as on one or more conditions under which one or more of these components may dry and polymerize. More specifically, one determinant of pore size formation is the differential evaporation rates of each component (e.g., the solvent and non-solvent). To control the evaporation rates of a polymer mixture, various incubation environments, such as temperature and solvent vapor concentration, may be modulated. Therefore, control over such conditions may allow optimization of a desired pore size and uniformity, and ultimately the performance characteristics of the films. In some examples, control over such conditions may allow for different regions of a film to have a different pore size as different drying conditions may be applied to different regions of a film allowing for uniform porosity in specific sections, but variance in the size of the pores between different sections of a film.

Specific pore sizes may be selected for a variety of reasons, such as the size and type of biomolecules to be bound, the size and type of ligand being used to perform the binding, the size and type of the label being used to identify the antigen, as well as the composition of the samples to be tested. In some embodiments, pore size may be modulated in different areas of a single nitrocellulose strip, allowing different groups of biomolecules to be detected in different areas of the strip. Additionally, the method disclosed herein may comprise a casting of the polymer mixture directly on a substrate and/or in a housing or reaction chamber by a robotic dispensing device, and thus eliminating multiple processing and assembly steps. Thus, manufacturing of LFA devices using hand-cutting of individual strip and installation of each strip onto a substrate housing may be reduced. In this way, it may be possible to improve manufacturing efficiency by depositing nitrocellulose film directly into or onto an assay device so that film strip cutting, functionalization, and assembly steps are reduced.

For example, the polymer mixture may be cast directly onto a flat substrate such as a silicon, glass or polymeric slide. The dried polymer mixture may be used for any purpose, in some examples, it will be used to analyze fluid samples for analytes of interest. In some examples, the flat substrate may be used to form a biochip (also referred to as a genome chip, DNA chip or gene array), comprising an array of miniaturized chemical or biological test sites such that many tests can be performed simultaneously. Such methods can be equally used with other point-of-care collection devices such as test strips, swabs, devices with reaction chambers with pre-deposited reagents, and the like.

While analytes of interest may bind directly to the nitrocellulose strips, in some examples, one or more microarrays of binding ligands may be printed on one or more locations of the nitrocellulose strip by itself or a nitrocellulose strip attached to a substrate and/or housing or reaction chamber. The microarrays of binding ligands may be used to capture biomolecules such as antibodies, antigens, oligonucleotides, and RNA for a variety of clinical and research purposes.

The nitrocellulose strip, comprising the microarray of binding ligands, may be incubated with patient samples of a bodily fluid including, but not limited to, saliva, blood, gingival crevicular fluid, serum, plasma, urine, nasal swab, cerebrospinal fluid, pleural fluid, synovial fluid, peritoneal fluid, amniotic fluid, gastric fluid, lymph fluid, interstitial fluid, tissue homogenate, cell extracts, sputum, stool, physiological secretions, tears, mucus, sweat, milk, semen, seminal fluid, vaginal secretions, fluid from ulcers and other surface eruptions, blisters, abscesses, and extracts of tissues including biopsies of normal and suspect tissues or any other constituents of the body which may contain the target molecule of interest. The assays described herein may further be used in the analysis of environmental samples such as water, plant or etymological exudates, foods, and the like.

As described herein, assessment of results may be qualitative or quantitative depending upon the specific method of detection employed. Thus, the sample may include a plurality of biomolecules, of which only a subset may be desired for analysis. As such, target biomolecules, which comprise the subset of sample biomolecules desired for analysis, may be tagged with a label. The label may be any type generally used for such purposes including, but not limited to, colorimetric, fluorescent and radioactive labels. In some examples, the fluorescent label may comprise organic dyes. The labels may conjugate directly to the biomolecule of interest, or may be chemically conjugated to a second binding ligand which may be the same or different than a binding ligand used to capture an antigen of interest. In some examples, the labels can be used to label multiple cell surface markers, increasing the likelihood of binding as well as the visibility of the biomarkers of interest, thereby increasing the accuracy of any test using this assay.

The fluorescent label may be any suitable fluorescent tag which may bind to the target molecules and emit light of a different wavelength than the light source in response to excitation from the light source, such as a laser. In some embodiments a plurality of fluorescent labels which emit light of different wavelengths may bind different target molecules, allowing a single biological sample to be tested for a plurality of biomarkers for a plurality of diseases or conditions. A biomarker may be a protein (including an antigen, antibody, antibody fragment, enzyme, or other polypeptide), an oligonucleotide (including DNA and RNA), a lipid, a carbohydrate, or other biomolecule whose presence is indicative of disease, infection, environmental exposure, contamination, etc., either alone or in combination with additional biomarkers. The printed microarray of binding ligands may be referred to as an assay once the biomolecules from the sample have been chemically bound to the wells or spots and have been tagged with the fluorescent label.

Non-limiting examples of assays that may be imaged by an imaging device of the present disclosure include those using reverse-phase protein arrays (RPPAs), antibody capture arrays, proteome arrays, and Western blots. It will be appreciated that while the examples described herein are with reference to a protein sample, this is not meant to be limiting, and that in alternate embodiments, the methods, assays, and devices described herein may be used with any of the alternate target analytes and biological molecules described above.

The RPPA may comprise cell or tissue lysates (complex mixtures of protein and other cellular components) deposited onto a porous nitrocellulose microarray surface in a denaturing lysis buffer. After microarray printing, a standard RPPA assay protocol may be performed utilizing available reagents, for example, those available in a kit.

Antibody capture microarrays may comprise an assortment of purified antibodies that recognize specific antigens. Antibodies are deposited individually, allowing for spatial recognition of bound antigens. Like an enzyme-linked immunosorbent assay (ELISA), antibody capture arrays may be designed to capture specific biomarker antigens from biological samples such as blood, serum, urine, cell lysates, food samples, or others, in a multiplexed fashion. Detection of positive array elements may depend on incorporation of a label molecule (such as a fluorescent semiconductor nanocrystal (QNC) or a fluorophore) and identification of the positive array element. Incorporation of the reporter molecule may be to the antigen itself (prior to performing the assay) or by indirect labeling of the antigen with another molecule (typically an antibody or antibody fragment). These additional molecules may also demonstrate specificity for individual biomarkers. This antibody or antibody fragment may have a fluorophore attached to it directly or may be labeled indirectly by a secondary antibody or via a biotin-streptavidin interaction with a fluorophore conjugated streptavidin. After microarray printing, an assay protocol can be performed with reagents found in a kit, such as the Protein Array Assay System kit from Grace Bio-Labs.

Proteome microarrays may comprise an assortment of purified or semi-purified proteins which are recognized by specific antibodies, proteins, or cofactors. Proteins may be deposited individually, allowing for spatial recognition of binding partners. Detection of positive array elements depends on incorporation of a label molecule (such as a fluorophore) and identification of positive array elements. Incorporation of the reporter molecule may be directly to the binding partner itself (prior to performing the assay) or indirectly by labeling the antigen or cofactor with another molecule (typically an antibody or antibody fragment). This antibody or antibody fragment may have the fluorophore attached to it or may be labeled via a secondary antibody or biotin-streptavidin interaction to incorporate the fluorophore. After microarray printing, a proteome assay protocol can be performed with reagents found in a kit.

A Western blot (also referred to as a protein immunoblot) is an analytical technique used to detect specific proteins in a given sample, such as a tissue homogenate or extract. Gel electrophoresis is used to separate native proteins by 3-D structure. Alternatively, denatured proteins may be separated by the length of the polypeptide using gel electrophoresis. Proteins may then be transferred to a membrane (typically nitrocellulose) where they are stained with antibodies specific to the target protein.

The nitrocellulose strip comprising the microarray which has been incubated with the patient or environmental sample and tagged with the fluorescent labels which bind to one or more biomarkers in a sample may be imaged using a microarray assembly. The microarray assembly may comprise a plurality of excitation sources and detection filters which may be manipulated to analyze the assay. The resulting images are then captured and analyzed to detect the presence or absence of analytes of interest and/or quantitatively assess the analytes of interest. The results may be analyzed by the microarray assembly and/or transmitted to the cloud or a nearby device for display via a user interface for analysis. In some examples, the plurality of excitation sources and detection filters may be automatically adjusted. In other examples, they may be manually adjusted. In a further example, they may be both manually and automatically adjusted. For example, gross manipulations of the position of the filters and/or lasers may be executed automatically and fine manipulation may be executed manually, or vice versa.

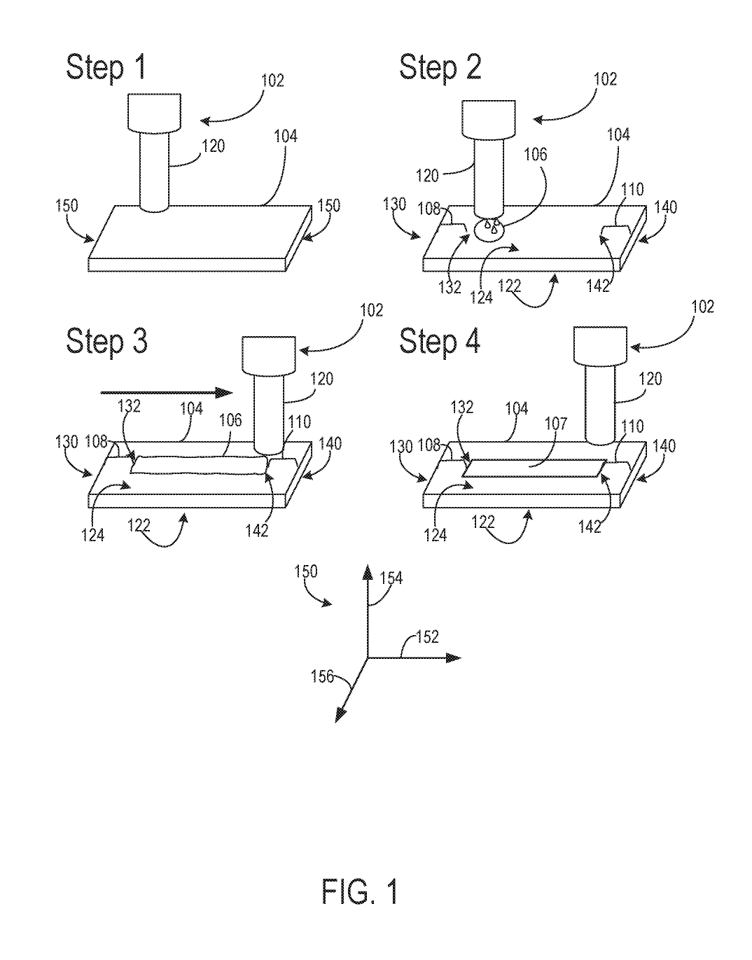

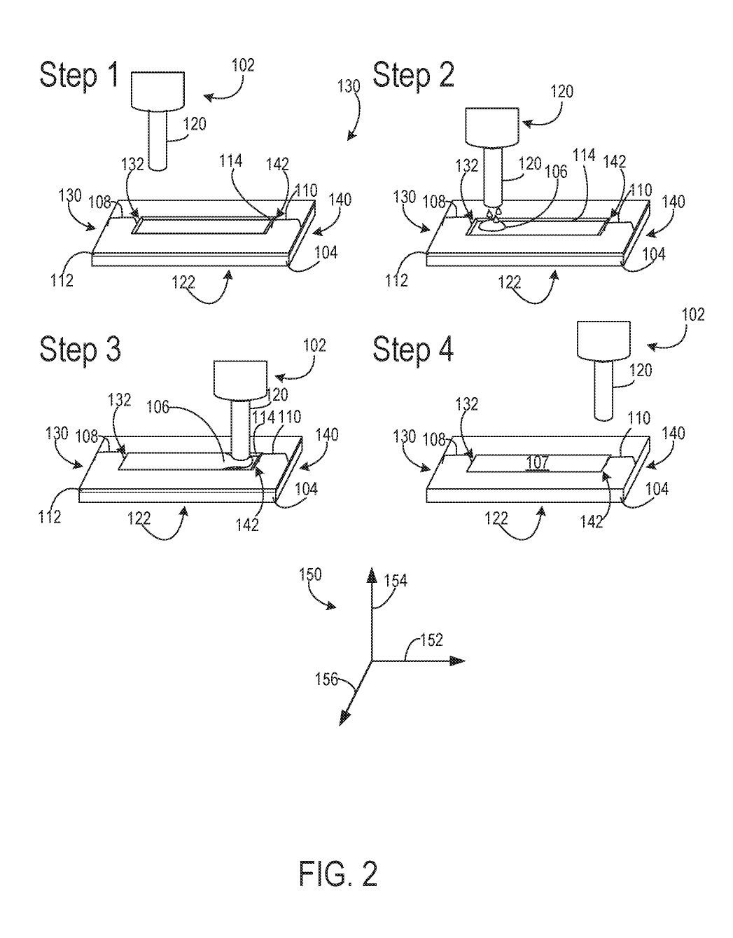

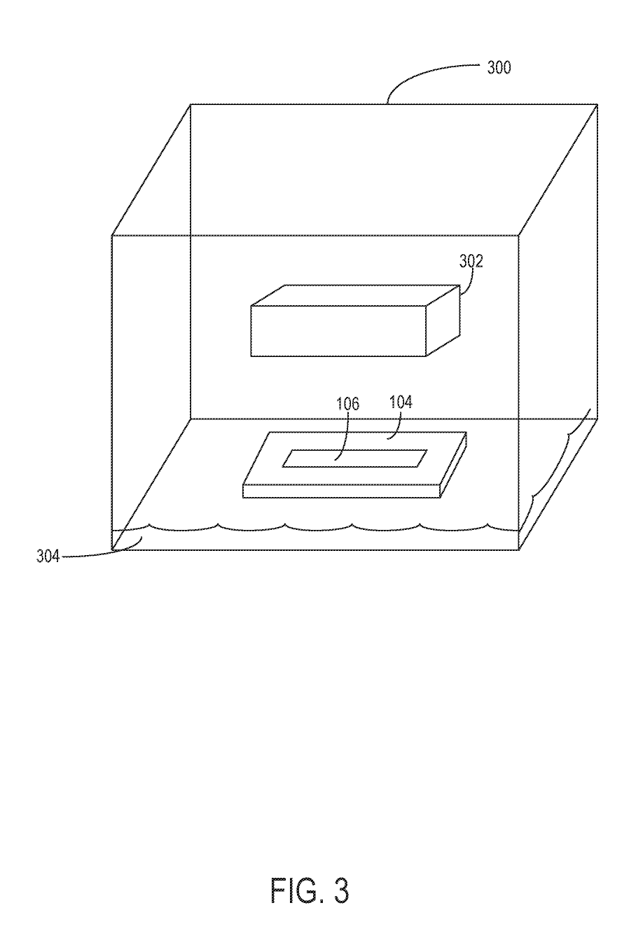

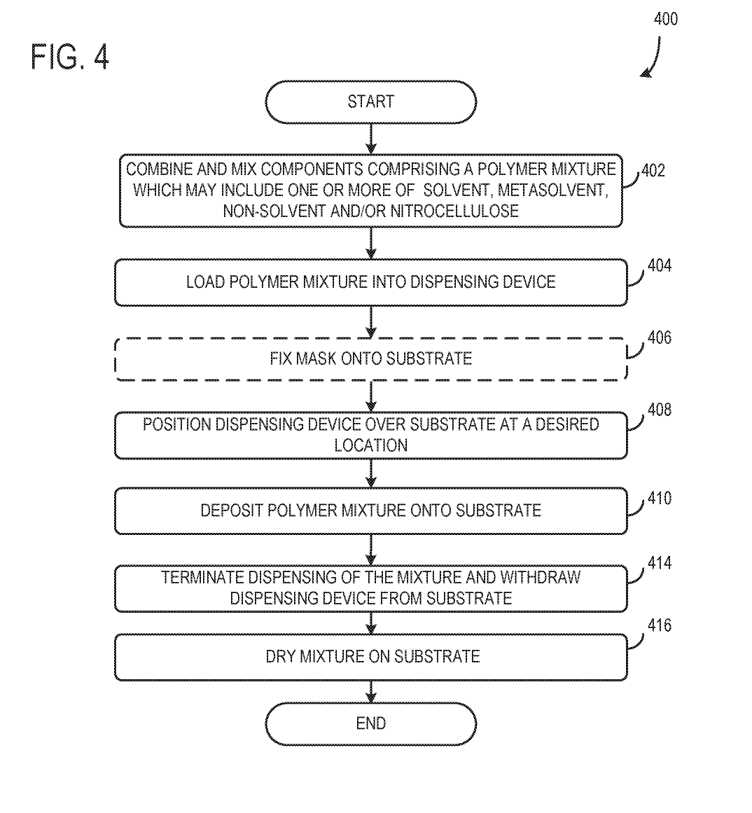

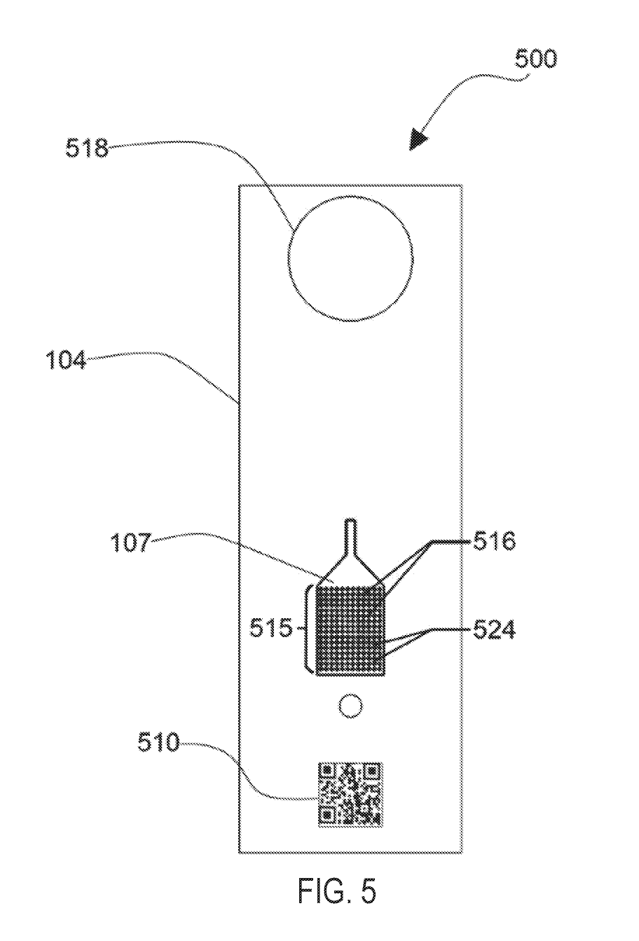

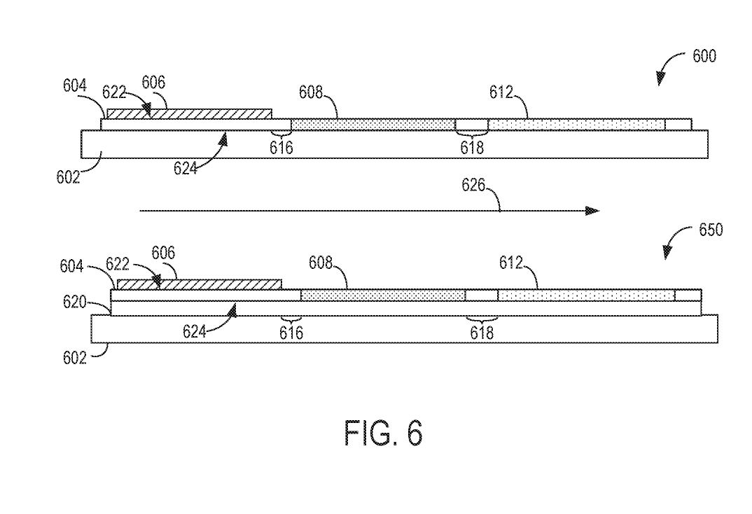

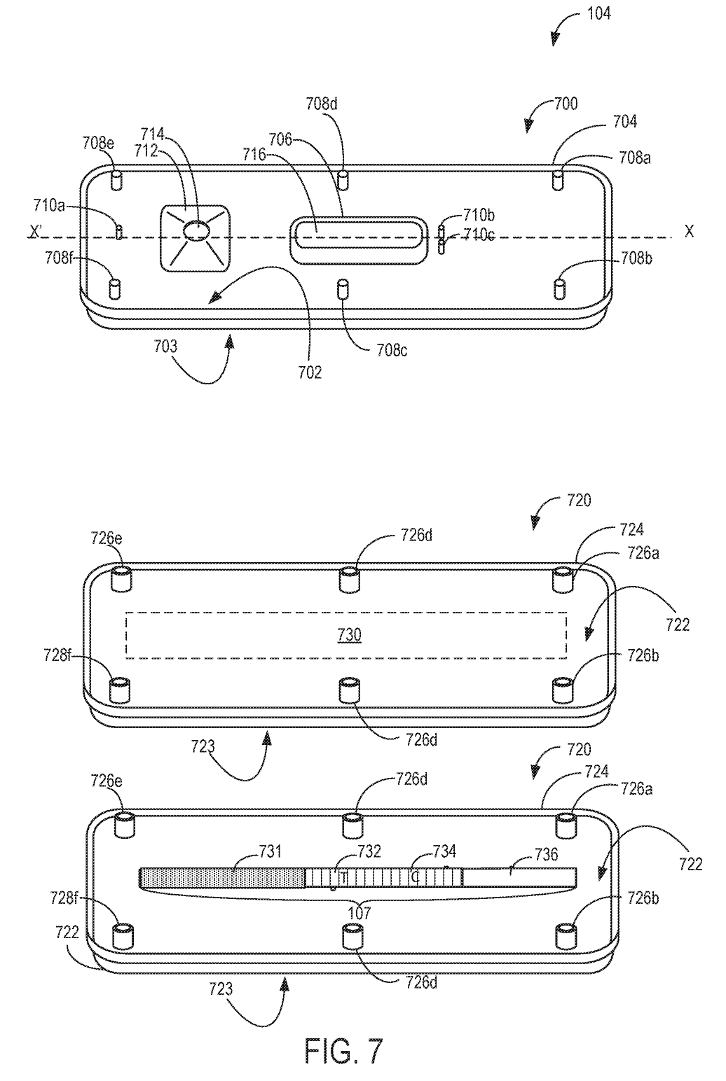

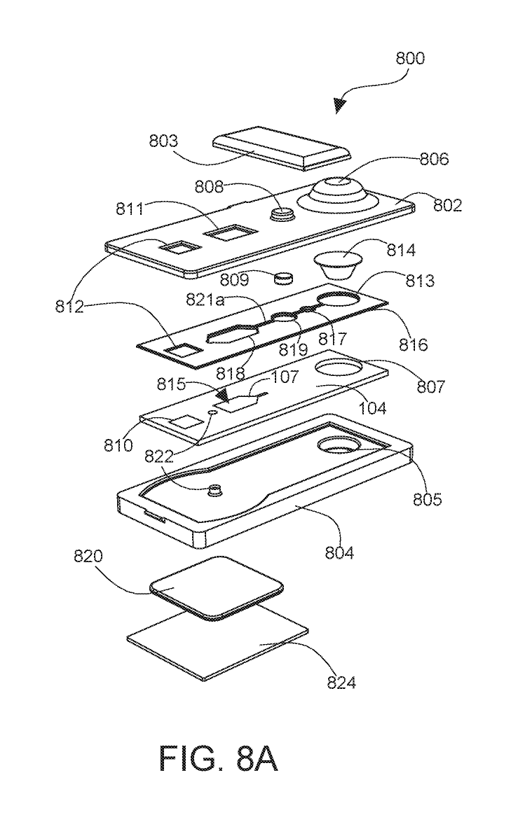

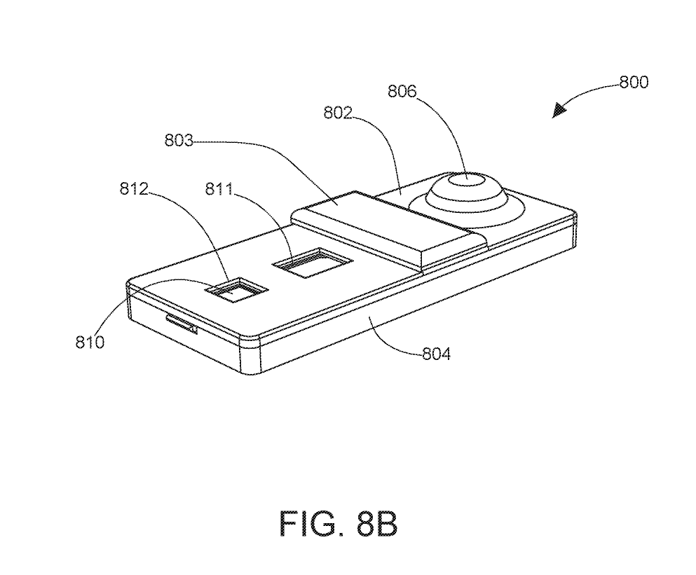

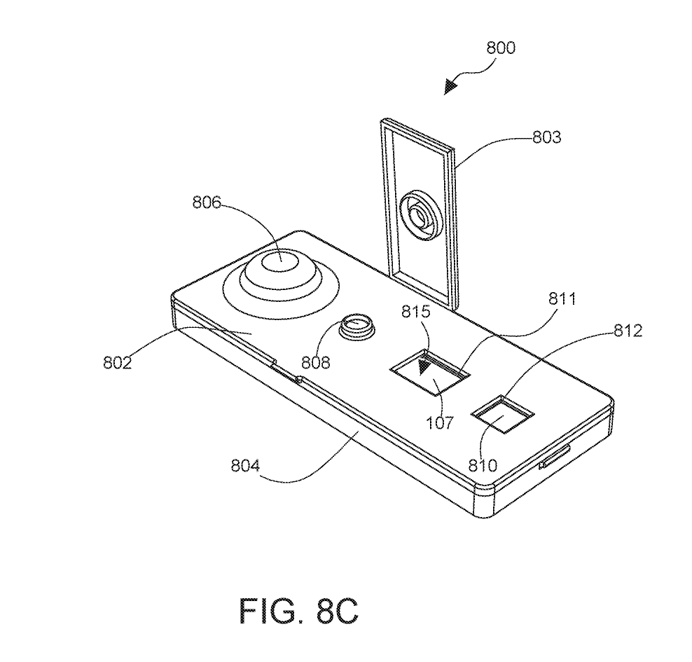

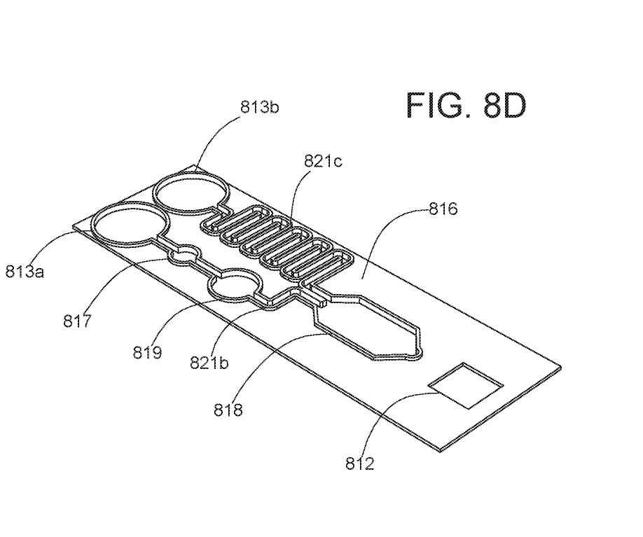

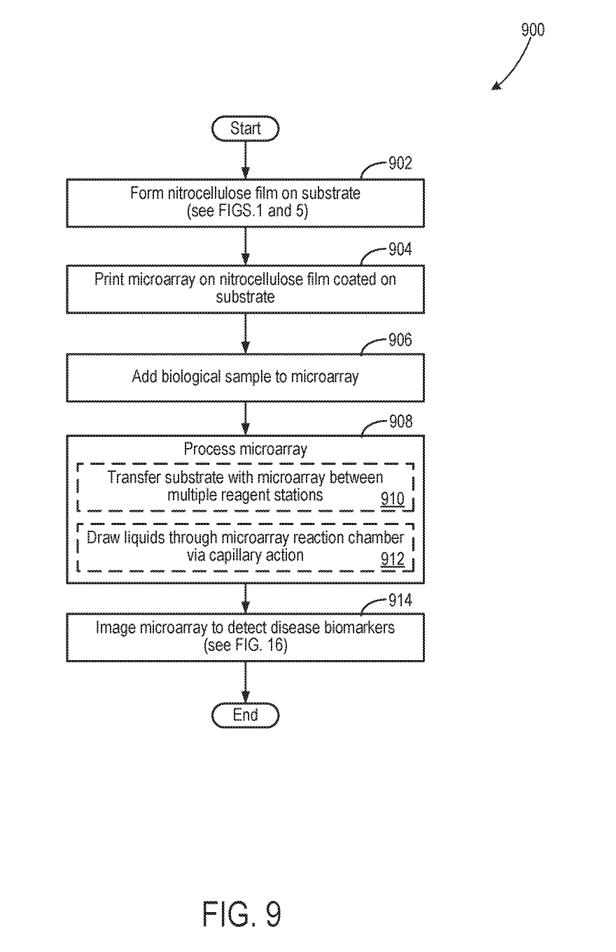

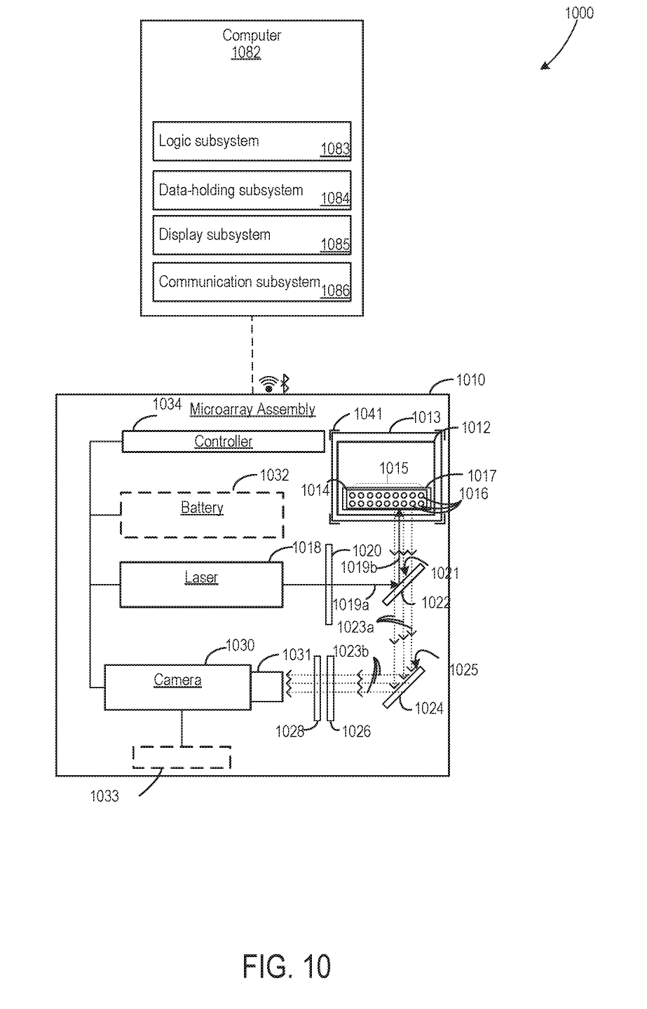

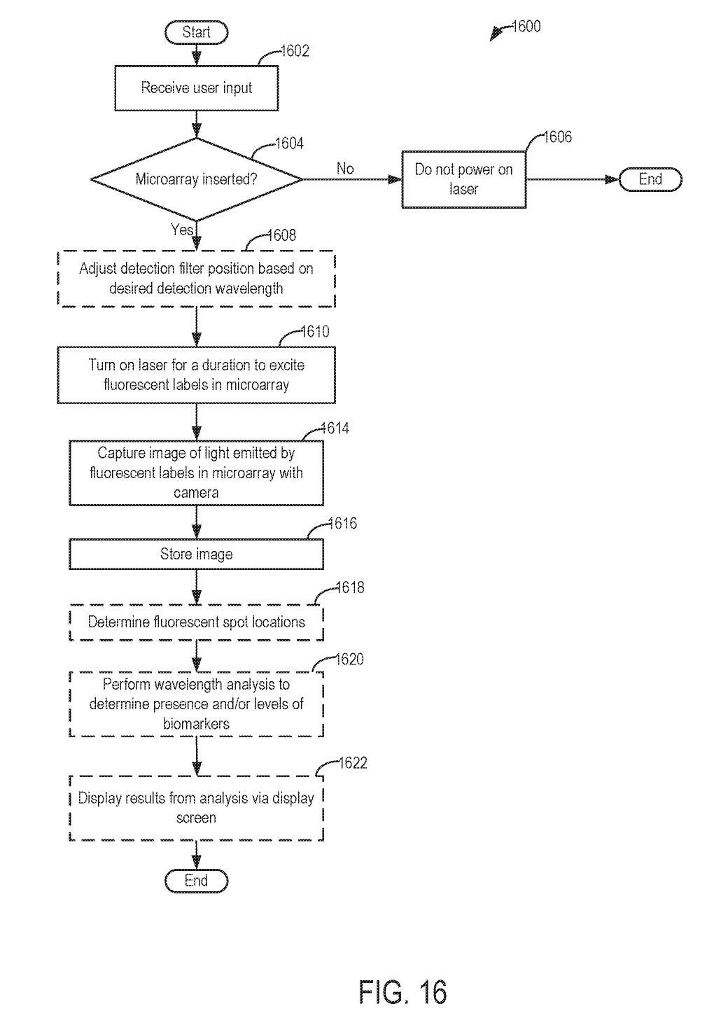

FIG. 1 shows an example device for depositing a polymer mixture in order to form a nitrocellulose-based strip. FIG. 2 shows a system similar to that shown in FIG. 1, but with an additional framed mask forming a well to produce a specific shape of a polymer mixture. FIG. 3 shows an example of a polymer mixture strip drying in a controlled chamber. FIG. 4 shows a flow chart of an example method for production of a lateral flow assay strip. FIG. 5 shows an enlarged view of an example of a substrate that may be included in the device of FIG. 1. FIG. 6 shows an example of a lateral flow assay strip that may be produced via the device of FIG. 1. FIG. 7 shows an example of a substrate that may be included in the device of FIG. 1. FIG. 8A shows the inclusion of a substrate in a cartridge that may be used with the device of FIG. 2. FIGS. 8B, 8C and 8D show a variety of components that may be used with the cartridge of FIG. 8A. The cartridge may additionally include liquid reagents for the lateral flow assay on-board. The lateral flow assay strip, which may also be referred to as a nitrocellulose strip herein, may additionally be printed with a microarray comprising a plurality of binding ligands and the microarray of binding ligands may be used to test a patient sample for one or more diseases according to the example method of FIG. 9. Specifically, the plurality of binding ligands may be each configured to bind to one or more target biomolecules (also referred to herein as biomarkers) in the patient or environmental sample, and the target biomolecules may be labeled for detection with one or more labels. Such labels may be colorimetric, fluorescent, radioactive, or other labels that allow for the identification and quantification of biomolecules of interest. For example, after fluorescently labeling the bound target biomolecules, the microarray of binding ligands bound to target biomolecules may be imaged using a biomolecule microarray assembly in a biomolecule analysis system, such as the systems described with respect to FIGS. 10-15, and according to the example method of FIG. 16.

FIGS. 1 and 2 show a dispensing device 102 for dispensing a polymer mixture 106 onto a substrate 104 to form a porous polymer strip 107 or lateral flow assay (LFA) strip 107 when dried. Specifically, FIGS. 1 and 2 show four sequential steps in the dispensing of the mixture 106 onto the substrate 104: step 1, followed by step 2, followed by step 3, and followed by step 4. Each of the steps will be described in greater detail below with reference to the description of FIGS. 1 and 2.

FIG. 2 shows an example where a mask 112 is positioned on top of the substrate, the mask 112 including a well 114, into which the polymer mixture 106 be dispensed. The mask 112 may also be referred to herein as cover 112. Thus, the well 114 may be a recess within the mask 112 that contains the mixture 106 within the interior of the volume it defines. As such, the shape and size of the porous polymer strip 107 may be more controlled, and may be defined by the well 114. The polymer strip 107 may therefore be sized to approximately the same size as the well 114. Said another way, the strip 107 may be fully contained within the mask 112. In this way, the uniformity of the shape and size of the polymer strip 107 from strip to strip may be increased, and variance in the shape and size of the polymer strip 107 may be reduced. Since the dispensing device 102 and substrate 104 are the same in FIGS. 1 and 2, components of the dispensing device 102 and substrate 104 introduced in FIG. 1 may not be reintroduced or discussed again in the description of FIG. 2.

In some examples, the liquid polymer mixture 106 is a mixture of nitrocellulose, solvent, and non-solvent. Thus, in the description herein, the liquid polymer mixture 106 may be referred to as liquid nitrocellulose matrix 106. In one embodiment, the solvent may dissolve the nitrocellulose, while the non-solvent may be miscible with the solvent at a given concentration, but may phase separate when the evaporation of the solvent causes the relative non-solvent concentration to exceed a certain threshold. Specifically, in one example, the liquid polymer mixture 106 comprises a higher relative solvent concentration such that the non-solvent and solvent are completely miscible and allow dissolution of nitrocellulose by said solvent. In addition, the solvent may be more volatile than the non-solvent, so that after a selected amount of time under controlled conditions, the solvent concentration decreases in the mixture due to differential evaporation. As the relative concentration of non-solvent increases beyond a critical threshold, a phase separation occurs, causing droplets of non-solvent to form within the solvent/nitrocellulose solution in the form of an emulsion. The evaporation of solvent also increases the nitrocellulose polymer concentration beyond a critical solubility threshold, at which point the mixture solidifies from the remaining solvent solution, causing emulsified non-solvent droplets within the solvent to form voids amongst the polymerized nitrocellulose. The droplets are the structural bases for the pores within the solidified nitrocellulose. In other words, the formation of pores as the polymer mixture dries is dependent on the differential evaporation rates of the solvent and non-solvent. Since the droplets of emulsified non-solvent will not contain any nitrocellulose, their size and distribution in the emulsion defines the size, shape, and distribution of the eventual pores in the film after all liquids are removed.

In some embodiments, the solvent for the nitrocellulose mixture includes one or more of acetone, methyl acetate, tetrahydrofuran, toluene, and propylene oxide. In other embodiments, the solvent may include another appropriate solvent. Appropriate non-solvents that are miscible with said solvents at certain relative concentrations but phase separate when the relative non-solvent concentration exceeds a critical threshold include, but are not limited to, water, butanol, ethanol, and isopropanol, and mixtures of these non-solvents. Unlike the solvents, the non-solvents do not cause solvation of the nitrocellulose.

In yet another embodiment, other components included in the polymer mixture of the device disclosed herein may comprise detergents, hydrophilic additives, plasticizers, and/or meta-solvents. According to the current disclosure, meta-solvents are liquids in which nitrocellulose is not soluble in a pure solution, but when combined with a solvent will allow for nitrocellulose solvation. For example, ethanol is not a solvent of nitrocellulose in pure form, but mixtures of ethanol and acetone are nitrocellulose solvents; therefore in combination with acetone, ethanol is considered a meta-solvent. Use of meta-solvents can alter the overall evaporation rate of the solvent, allowing manipulation and control over the rate of relative solvent/non-solvent concentration changes, and thus the polymer film pore size.

In one embodiment, one of a grade or type of nitrocellulose may be varied such that physical and chemical features of the resulting nitrocellulose-based strip may be optimized. Generally, nitrocellulose is graded according to its solution viscosity under certain sets of conditions. Viscosity is related to polymer chain length, in which larger chain lengths afford a higher viscosity solution in standard conditions, which is described by the designation of time for a weight to travel a set distance through the solution (in seconds). In one embodiment, grades of nitrocellulose used may include ½ second, 15-30 second, 30-40 second, and/or 125-175 second to achieve a desired viscosity of the resulting nitrocellulose-based strip. In other embodiments, mixtures of different grades of nitrocellulose may be combined to create blends that achieve certain desired performance aspects, such as controlled pore sizes, and/or controlled liquid flow rates.

FIG. 1 shows a device and system wherein a strip of mixture 106 is cast directly onto the substrate 104 by the dispensing device 102. The substrate 104 may be a planar or non-planar solid structure, composed of solid material including, but not limited to, glass, metal, plastic, or other materials. In one embodiment, the dispensing device 102 may controllably distribute the polymer mixture 106 in a defined manner via spray coating, syringe extrusion, or slot dye coating. For example, dispensing device 102 may be a hypodermic and/or square-tip syringe needle. In another embodiment, the dispensing device 102 may comprise a multi-directional dispensing head 120 that may be moved/translated along a horizontal axis 152, lateral axis 156, and vertical axis 154. The dispensing head 120 and dispensing device 102 may in some examples be physically coupled to one another and as such may be moved together. However, in other examples, the dispensing head 120 and dispensing device 102 may not be physically coupled to one another, and as such the dispensing head 120 or dispensing device may be moved without movement of the other. The dispensing head 120 may be any suitable device for dispensing the polymer mixture 106 such as a nozzle, injector, syringe pump, etc.

Axis system 150 includes the horizontal, lateral, and vertical axis, 152, 156, and 154, respectively. The lateral axis 156, horizontal axis 152, and vertical axis 154, may be orthogonal to one another, and as such may define a three dimensional coordinate system. Thus, the dispensing head 120 and dispensing device 102 may be movable in the planes defined by the axis system 150. As such, the dispensing device 102 may be movable within planes parallel to a first plane defined by the lateral axis 156 and horizontal axis 152. Further, the dispensing device 102 may be movable within planes parallel to a second plane defined by the horizontal axis 152 and vertical axis 154. The dispensing device 102 may further be movable along planes parallel to a third plane defined by the lateral axis 156 and vertical axis 154.

The dispensing device 102 may further include a pump (not shown in FIG. 1) that is compatible with the mixture 106, for pressurizing and delivering the mixture 106 to the dispensing head 120.

The dispensing device 102, and more specifically, the dispensing head 120, may be moved along any of the axis, 152, 154, and 156, by an actuator (e.g., electromechanical robotic arm), not shown in FIG. 1. Thus, the actuator may adjust the position of the dispensing device 102 and dispensing head 120 above the substrate 104. The actuator may adjust the position of the dispensing device in response to signals received from a controller, the controller having computer readable instructions stored in non-transitory memory for controlling the dispensing device 102. Thus, in the description herein, any movement or change in position of the dispensing device 102 and/or dispensing head 120 may be achieved by the actuator in response to signals received from the controller, and/or inputs from a device operator.

For example, at step 1 of FIG. 1, the dispensing head 120 is positioned over the substrate 104. Specifically, the dispensing head 120 is positioned more proximate a first end 130 of the substrate 104 than a second end 140 of the substrate. The dispensing head 120 may be positioned a vertical distance above (e.g., in the positive direction along vertical axis 154) relative to the substrate 104. In some examples, the dispensing head 120 may be positioned a threshold vertical distance above the surface of the substrate 104. Specifically, the dispensing head 120 may be positioned the threshold vertical distance above a top surface 124 of the substrate. The threshold vertical distance may be a distance in a range of distances between 0.2-5.0 mm. As shown in FIG. 1, the first end 130 may be parallel to the second end 140 of the substrate 104, where the ends 130 and 140 may be displaced relative to one another along the horizontal axis 152. Put more simply, the ends 130 and 140 may define the physical extent of substrate 104 along the horizontal axis 152. In this way, the second end 140 may be positioned to the right, or in the positive direction along the horizontal axis 152, relative to the first end 130 of the substrate 104.

Further, the substrate 104 may include a bottom surface 122 opposite a top surface 124. The bottom surface 122 and top surface 124 may define the extent of the substrate 104 along the vertical axis 154. As shown in FIG. 1, the bottom surface 122 may be parallel to the top surface 124 of the substrate 104, where the surfaces 122 and 124 may be displaced relative to one another along the vertical axis 154. Specifically the top surface 124 may be vertically above the (e.g., displaced in the positive direction along the vertical axis 154) relative to the bottom surface 122. Thus, the top surface 124 may be oriented so that it faces the dispensing head 120, and the bottom surface 122 may be oriented so that it faces away from the dispensing head 120.

The dispensing head 120 may be programmed to move along the horizontal axis 152, such that its motion defines the desired shape and size of the strip 107. Such motion allows the resulting wetted substrate area to be much larger than the viscosities and contact angles formed by the polymer mixture alone would naturally allow. Dispensing of the solution may be performed intermittently in a single pass or multiple passes, or continuously depending on a desired outcome.

Although the depicted embodiment in FIG. 1 shows one dispensing device with a single dispensing head to produce one individual strip, it should be appreciated that in other examples, the dispensing device may comprise a multi-channel syringe-like dispensing head and one or more pumps to perform high accuracy, multi-channel dispensing. In one embodiment, the dispensing head comprises an array of flat hypodermic syringe needles. In this way, the system may increase its capacity and efficiency to produce a plurality of liquid polymer strips in one or more passes and runs of the system.

At steps 2 and 3, a mixture 106 is then ejected from the dispensing head 120 of the dispensing device 102 onto the substrate 104. The mixture 106 may be dispensed from the dispensing head 120 vertically downward, or in the negative direction along the vertical axis 154. Thus, the mixture 106 may travel in a substantially straight line, parallel to the vertical axis 154, in a downward direction (e.g., negative direction of vertical axis 154). Specifically the mixture 106 may be ejected onto the substrate beginning at a first position 132 of the substrate 104 to a second position 142 on the substrate 104. Thus, in steps 2 and 3, the dispensing head 120 may be moved along the horizontal axis 152 from vertically above the first position 132 to vertically above the second position 142. Dispensing of the mixture 106 may begin at the first position 132, continue as the dispensing head 120 is moved in the positive direction along the horizontal axis 152, and may then terminate when the dispensing head 120 reaches the second position 142. The first position 132 may be a location on the substrate 104 positioned a first distance 108 away from the first end 130 of the substrate 104. Further, the second position 142 may be a location on the substrate 104 positioned a second distance 110 away from the second end 140 of the substrate. In some examples, the first distance 108 and second distance 110 may be substantially the same. However, in other examples, the first distance 108 may be greater or smaller than the second distance 110. Thus, the first position 132 and second position 142 may define the physical extent of the dispensing region of the mixture 106 and may therefore define the length of the resulting nitrocellulose matrix.

The length of the nitrocellulose matrix may be adjusted by adjusting the first distance 108 and/or second distance 110. Thus, the dispensing of the mixture 106 may be configured to begin closer to or further away from the first end 130 of the substrate 104, and may be configured to end closer or further away from the second end 140 of the substrate, depending on a desired length of the nitrocellulose matrix, LFA device, and appropriate substrates.

In this way, the mixture 106 may begin dispensing onto the substrate 104 at the first position 132 via the dispensing head 120, when the dispensing head 120 is positioned vertically above the first position 132. The mixture 106 may continue to be dispensed as the dispensing head 120 is translated along toward the second position 142, away from the first end 130. In response to the dispensing head 120 reaching the second position 142, dispensing of the mixture 106 may be terminated.

At step 4 of FIG. 1, the polymer mixture 106 is allowed to dry to form a porous polymer strip 107. Thus, the porous polymer strip 107 may comprise the same compounds and elements as the mixture 107, but may be in solid physical state instead of a liquid state. Said another way, the strip 107 may be the same as the polymer mixture 106 except in a solid state instead of a liquid state. As such, the strip 107, may be referred to as solid nitrocellulose matrix strip 107 since the polymer mixture 106 comprises a nitrocellulose matrix. Therefore, the length of the polymer strip 107 may extend from the first position 132 to the second position 142 along the horizontal axis 152.

In yet another embodiment, a temperature control element, such as a water-cooled or heated plate (not shown), may be included to control the temperature of the substrate during the drying process. Lower or higher temperatures provided to the substrate may reduce or enhance the drying rate depending on desired conditions, and thus may serve to increase the porosity and uniformity of the strip 107 from piece-to-piece.

FIG. 2 shows an embodiment of the dispensing device 102 and substrate 104 shown above with reference to FIG. 1, with a framed mask 112 positioned on top of the substrate 104. Thus, the framed mask 112 may be positioned such that it is in one or more of face sharing, physical, and/or sealing contact with the top face 124 (shown above with reference to FIG. 1) of the substrate 104, and may be positioned between the substrate 104 and the dispensing device 102. Thus, the mask 112 may be physically coupled to the substrate 104, on the top face 124 of the substrate 104, where the top face 124 of the substrate 104 faces the dispensing device 102. The framed mask 112 may include a well 114 into which the mixture 106 is dispensed, for one or more of retaining, forming, and/or shaping the mixture 106 as it is dispensed from the dispensing device 102 and cools to form the strip 107. Thus, the steps shown above with reference to FIG. 1 for dispensing the mixture 106 may be same in FIG. 2, except that instead of the mixture 106 being dispensed directly onto the substrate 104, the mixture 106 may be dispensed onto the framed mask 112. Said another way, the mixture 106 may be dispensed into the well 114 in the same or similar manner to that described above with reference to FIG. 1 for dispensing the mixture 106 directly onto the substrate 104. As such, the well 114 may be sized such that it extends from the first end 130 to the second end 140. By including the framed mask 112 on the substrate 104, the shape, size, and other features of the strip 107 may be adjusted and/or controlled to a greater degree of accuracy. For example, the mask 112 may slow down the drying rate of the polymer mixture and thereby produce strips with increased uniformity. Furthermore, the thickness of the mask 112 and shape and size of well 114 may be varied to adjust the shape, thickness, and size of the wet polymer mixture strip. However, the thickness of the final polymer film may also depend on the nitrocellulose percent composition and relative porosity of the resulting strip.

In the embodiment shown in FIG. 2, the mask 112 is generally rectangular in shape and spans the majority of the substrate 104 along the horizontal axis 152 and lateral axis 156. In other embodiments, the shape and size of the mask 112 may be different than depicted in FIG. 2, and may comprise other various shapes and sizes. Specifically the mask 112 may be sized to approximately the same size as the top face 124 of the substrate 104. Moreover, the mask 112 may be made of silicone rubber or other appropriate materials. Use of materials such as silicone rubber ensures that an adequate seal is formed between the mask 112 and the substrate 104. In one example, the mask 112 is approximately 2-4 mm thick and spans generally across the substrate. In sum, the provision for the mask allows for defined deposition of polymer strips, manipulation of the final shape and placement of the polymer strip onto a substrate, and the option to deposit into non-planar three dimensional substrates.

The well 114 may be formed by a cut-out portion of the mask 112. In other examples, the well 114 may be included in the substrate 104, and may form a recess within the substrate 104. As such, the depth of the well 114 may be sized up to the thickness of the mask 112. As such, in some examples, the depth of the well may be in a range of depths, up to 4 mm. The well 114 may fully contain the mixture 106 as it is dispensed from the dispensing head 120. Thus, the well 114 may serve as a container in which the mixture 106 may dry and form the strip 107. As such, the strip 107 may only be exposed on one surface. In some examples, all of the mixture 106 dispensed by the dispensing head 120 may be contained within the volume enclosed by the well 114, and substantially none of the mixture 106 may extend beyond the well 114. In this way, the shape of the strip 107 may conform to the shape/contour of the well 114. As such, the shape and/or size of the well 114 may be adjusted to produce a desired shape and/or size strip. In this way, the strip 107 may be approximately the same size and shape as the volume enclosed by the well 114. However, in other examples, the shape and/or size of the strip 107 may be different than that of the well 114.

The dispensing head 120 may be positioned over the well 114, and the mixture 106 may be dispensed into the well 114. In some examples, the dispensing head 120 may remain stationary while dispensing the mixture 106. However, in other examples, the dispensing head 120 may be moved along the horizontal axis 152 in the same or similar manner to that described above with reference to FIG. 1 when dispensing the mixture 106. For example, the dispensing head 120 may move from vertically above the first position 132, to vertically above the second position 142 while dispensing the mixture 106. The mixture 106 may accumulate in the volume enclosed by the well 114 as it is dispensed into the well 114. For example step 3, depicts how the volume of mixture 106 in the well 114 has increased relative to step 2, as more mixture 106 is added to the well 114 during dispensing. Dispensing of the mixture 106 may terminate when the dispending head 120 reaches the second position 142. However, in other examples, the dispensing may terminate in response to a volume of the mixture 106 reaching a threshold volume within the well 114, and/or a liquid level in the well 114 reaching a threshold level. In some examples, the mixture 106 may be dispensed into the well 114 until substantially the entire volume enclosed by the well 114 is full of the mixture 106. However, in other examples, the dispensing the mixture 106 may stop when the mixture 106 occupies less than the entire volume enclosed by the well 114.

In this way, the liquid nitrocellulose mixture 106 may be dispensed by a dispensing device 102 onto a substrate 104 to form a solid nitrocellulose matrix strip 107. A dispensing head 120 of the device 102 may be moved over the substrate 104 while dispensing the mixture 106 to increase the uniformity of dispersal of the mixture 106 on the substrate 104. Further, a mask 112 including a well 114 may be positioned on top of the substrate 104, where the well 114 may be configured to receive and retain the mixture 106 dispensed by the dispensing head 120. Thus, the mixture may, in some examples, be dispensed into the well 114. As such, a desired shape and/or size of the matrix strip 107 may be achieved by adjusting the shape and/or size of the well 114 to match the desired shape and/or size. In this way, after being dispensed and collected in the well 114, the mixture 106 may conform to the shape and/or size of the well 114. Thus, as the mixture 106 dries and solidifies to form the matrix strip 107, the matrix strip 107 may take on the shape and or size of the well 114.

FIG. 3 illustrates a chamber 300 into which the substrate 104 and mixture 106 may be placed for casting and drying of the mixture 106. The chamber 300 may include 6 walls which fully enclose an interior volume of the chamber 300 in which the substrate 104 is positioned. Thus, after dispensing the mixture 106 on the substrate 104, such as after step 3 shown above with reference to FIGS. 1 and 2, the substrate 104 including the mixture 106 may be placed within the chamber 300 for drying, solidifying, and casting of the mixture 106.

However, in other examples, one or more of a nitrocellulose dispensing apparatus (e.g., dispensing device 102 shown in FIGS. 1 and 2) and the substrate 104 may be positioned and fully enclosed within the chamber 300, such that the nitrocellulose mixture may be deposited onto the substrate 104 within the chamber 300. In this way, the nitrocellulose mixture may be exposed to the environment within the chamber 300 during deposition and subsequent drying. As such, the environment within the chamber 300 may be adjusted to regulate the drying rate of the mixture. Thus, the process of solidifying the mixture 106 into the strip 107 may occur in the chamber 300.

In some embodiments, the chamber 300 may comprise controls that regulate temperature, vapor content, humidity, etc. For example, the chamber 300 may include a heater 302, which may heat and accelerate the drying process of the mixture 106. In other examples, an air conditioner, dehumidifier, and/or humidifier may be included in the chamber 300 for adjusting the temperature, humidity, etc. of the chamber 300. In this way, the rate at which the mixture 106 solidifies may be adjusted to a desired rate, where the desired rate may be determined based on a desired composition of the strip 107. Specifically, the desired rate may be determined based on a desired pore size and/or pore concentration of the strip 107. Thus, the rate at which the mixture 106 solidifies may be adjusted by adjusting one or more of the temperature and/or humidity of the chamber 300, to achieve the desired rate. In this way, one or more of a desired pore size, distribution, concentration, etc. may be achieved. For example, power supplied to the heater 302 may be increased to increase the drying rate of the mixture 106, and thus increase the density of pores formed during the drying of the mixture 106. As such, operation of the heater 302 may be adjusted to adjust the drying rate of the mixture 106, and therefore the pore size and/or distribution of pores in the strip 107. In other examples, the operation of the heater 302 may be adjusted to different positions such that different sections of the mixture 106 may be dried at different rates, creating multiple sections of different pore sizes on a single strip 107.

In still further examples, one or more of the temperature and/or humidity in the chamber 300 may be differentially controlled across a length and/or width of the chamber 300. Said another way, the temperature and/or humidity in the chamber 300 may not be uniform in some example. Thus, the strip 107 may be exposed to a gradient of temperature and/or humidity, resulting in a gradient distribution of pore sizes within the film, depending on the position of the strip 107 in the chamber 300.

In yet further examples, a reservoir 304 may be included within the chamber 300. The reservoir 304 may include one or more of solvents such as water and/or acetone. A volume and/or composition of the reservoir 304 may be adjusted to adjust the vapor concentration in the chamber 300. By adjusting one or more of the volume of the reservoir 304 and/or relative amount of different solvents included in the reservoir 304, the drying rate of the mixture 106 may be adjusted. In one example, a ratio of 1:4 acetone to water may be used in the reservoir 304. However, in other examples, the ratio may be greater or less than 1:4. Thus, by adjusting the relative amounts of different solvents in the reservoir 304, the drying rate of the mixture 106 may be adjusted to achieve the desired rate. As such, one or more of a desired pore size, distribution, and density may be achieved by adjusting the volume and/or composition of the reservoir 304.

FIG. 4 depicts an example method 400 for forming a polymeric nitrocellulose matrix strip (e.g., strip 107 shown in FIGS. 1-2 and 5-8), wherein a defined area of porous nitrocellulose is deposited onto a substrate (e.g., substrate 104, 500, or 602 shown in FIGS. 1-3 and 5-8D) by a dispensing device (e.g., dispensing device 102 shown in FIGS. 1-2). In some examples, a controller with non-transitory memory may include computer readable instructions for executing the method 400. As such, in some examples, the method 400 may be performed by the controller.

Method 400 begins at 402, which comprises combining and mixing components comprising a polymer mixture (e.g., mixture 106 shown in FIGS. 1-3) where the components may include one or more of a solvent, metasolvent, non-solvent, and/or nitrocellulose. The components of the polymer mixture may further contain, but may not be limited to, various grades and types of plasticizers and detergents. The method at 402 may further comprise determining desired amounts, volumes, types, and/or grades of the components to be mixed at 402. Thus, the desired amount/volume and type/grade of each of the components mixed at 402 may be determined and combined with one another to form the mixture.

After combining and mixing the components of the mixture at 402, method 400 may continue to 404, which may comprise loading the mixture into a dispensing device (e.g., dispensing device 102 shown in FIGS. 1-2). As described above with reference to FIGS. 1 and 2, the position of the dispensing device may be adjusted in three-dimensional space.

In some examples, method 400 may continue from 404 to optional step 406, which comprises fixing a mask (e.g., mask 112 shown in FIG. 2) onto the substrate. In some examples, an adhesive may be applied between the mask and the substrate. However, in other examples, other coupling techniques, such as fasteners and thermal bonding, may be used to adhere the mask to the substrate. In other examples, method 400 may proceed directly to 408 from 404, without fixing the mask onto the substrate at 404. Thus, in some examples, the mask may not be included on the substrate.

Method 400 may then proceed from either step 404 or 406 to 408, which comprises positioning the dispensing device over the substrate at a desired starting location (e.g., first position 132 shown in FIGS. 1 and 2. In some examples, the method 400 may comprise positioning the dispensing device over a well (e.g., well 114 shown in FIG. 2) included in the mask. The desired starting location may be approximately between 0.2 mm to 5.0 mm from the surface of substrate.

Once the dispensing device is positioned over the desired location of the substrate, the method 400 may continue to 410, which comprises depositing the polymer mixture onto the substrate. Dispensing the mixture may include supplying current to an electromechanical injector or valve in the dispensing device to dispense the mixture onto the substrate. In examples where method 400 performs step 406 and the mask is included, dispensing the mixture may comprise dispensing the mixture onto the mask, and specifically into the well included in the mask. Further, method 400 at 410 may include moving the dispensing device across the longitudinal axis of the substrate. Thus, the controller may send signals to an actuator of the dispensing device to move the dispensing device in a substantially straight line from the desired starting location across the longitudinal axis of the substrate to a desired end location (e.g., second position 142 shown in FIGS. 1 and 2), where the desired end location may be horizontally displaced from the starting location. Further, the vertical positioning of the dispensing device may be maintained. Said another way, the distance between the dispensing head and the substrate may be maintained while moving the dispensing head from the starting location to the end location.

In response to the dispensing head reaching the end location, method 400 may continue from 410 to 414, which comprises terminating the dispensing of the mixture and withdrawing the dispensing device from the substrate. Thus, an injector or valve of the dispensing device may be closed at 414, so that the mixture ceases to flow out of the dispensing device. Withdrawing the dispensing device may comprise moving the dispensing device so that the vertical distance between the dispensing device and the substrate is increased.

Method 400 may then continue from 414 to 416, which comprises ending the dispensing of the mixture and drying the mixture on the substrate. Thus, the method 400 at 416 may comprise solidifying the mixture, or said another way, changing the phase of the mixture from liquid to solid. In some examples, the mixture may be placed in a sealed chamber (e.g., chamber 300 shown in FIG. 3) to increase or decrease the rate at which the mixture dries and/or solidifies. As discussed above, the formation of pores as the polymer film dries may be dependent on the differential evaporation rates of the solvent, meta-solvent, and non-solvent. Therefore, control over the evaporation conditions may be adjusted at 416 to regulate the pore formation and the performance characteristics of the films.

To control the evaporation rates of the films, various incubation parameters, such as temperature, local vapor concentration above the mixture, and a presence of a framed mask may be controlled. For instance, in a condition in which a certain vapor pressure is desired and when using a system comprising the combination of acetone, ethanol, and water, an incubation chamber (such as chamber 300 of FIG. 3) with a reservoir of approximately 20% acetone and 80% water may be provided during the drying process. These conditions may result in the local environment above the polymer mixture containing a specific fraction of acetone vapor that results in large and uniform pore sizes in the nitrocellulose strip. Alternatively, the relative humidity in the chamber can be manipulated to provide an environment that causes controlled evaporation of solvent and precipitation and/or solidification of polymer from solution.

In addition, a temperature at the substrate may affect the evaporation rate, so that modulation of temperature during drying of the polymer mixture at 416 may result in desirable outcomes. For example, again using the acetone, ethanol, and water solvent system described above, casting at 55° F. (e.g., 12-13° C.) may provide strips (e.g., strip 107 shown in FIGS. 1-2, 5, 7 and 8) with an appropriate fluid flow rate and larger pore sizes. Depending on the solvent systems used and the relative differential evaporation rates of solvent, meta-solvent, and non-solvent, casting at increased temperatures can result in films with smaller pores, resulting in slower liquid capillary motion. The drying temperature of a substrate can be manipulated by controlling the temperature of the substrate bed. Therefore, by introducing a temperature gradient across the substrate bed, differential relative evaporation rates of solvent from a film strip can be induced, resulting in different pore sizes in different areas of a single strip. Varying and optimizing the temperature across the deposited area during method 400 may also comprise one aspect of step 416.

In this way, one or more of the vapor concentration, temperature, etc., may be adjusted at 416 to increase or decrease a size of pores formed during drying and solidifying of the mixture. By regulating the formation and/or size of pores in the mixture as it dries to form the strip, different concentrations and/or sizes of pores may be formed in different areas of the strip, which can result in different differential flow rates within the strip. In this way, flow rates across the strip may be adjusted by controlling the location, concentration, and/or size of the pores, where formation and/or size of the pores may be adjusted by increasing and/or decreasing one or more of the temperature and/or vapor concentration of the environment where the mixture is dried at 416. Specifically, by increasing the temperature, the evaporation rate of the solvent may be increased, resulting in reduced solubility of the non-solvent, which may lead to the formation of emulsified non-solvent droplets, and thus increased pore density. In further examples, decreasing the humidity may increase the evaporation rate of the solvent, thereby increasing pore density. Thus, the drying and polymerizing at 416 may comprise one or more of increasing the temperature and/or reducing the humidity to increase pore density.

Therefore, one or more of solvent/non-solvent composition and relative concentrations, nitrocellulose composition and concentration, additive composition and concentration, and environmental conditions including temperature, humidity, vapor pressure, air flow, may be adjusted to adjust one or more of pore size, density, and distribution. By adjusting the pore size, density, and/or distribution, flow characteristics of the strip may be adjusted.

Specifically, the method 400 at 416 may include increasing the temperature in response to one or more of an increase in a desired pore density and/or a decrease in desired pore size. Additionally or alternatively, the method 400 at 416 may include decreasing the temperature in response to one or more of a decrease in the desired pore density and/or an increase in the desired pore size. The temperature may be increased by increasing power supplied to a heater (e.g., heater 302 shown in FIG. 3). Conversely, the temperature may be decreased by decreasing power supplied to the heater.

Additionally or alternatively, the method 400 at 416 may include decreasing the humidity in response to one or more of an increase in the desired pore density and/or a decrease in the desired pore size. Further, the method 400 at 416 may include increasing the humidity in response to one or more of a decrease in the desired pore density and/or an increase in the desired pore size.

More simply, the drying rate of the mixture may be adjusted by adjusting one or more of the ambient temperature and/or humidity of the chamber in which the mixture dries. As such, one or more of the size, distribution, and density of the pores may be adjusted by adjusting one or more of the ambient temperature and/or humidity of the chamber. Following 416, method 400 ends.

From the above description, it can be understood that the system and method disclosed for production of lateral flow assays have several advantages, namely the reduction of process and assembly steps resulting in increased efficiency, reduced production costs, and increased value of final product. Specifically, by forming a nitrocellulose matrix strip on a substrate, processes such as cutting of the strip may be eliminated. By dispensing a liquid mixture of the nitrocellulose matrix into a well formed on the substrate, the shape of the resulting strip may be configured to any desired shape by adjusting the shape of the well. In this way, the constancy and repeatability of producing such strips may be increased.

Further by regulating the temperature and/or humidity during drying and/or solidifying the liquid mixture into the strip, a temperature and/or humidity to which the mixture is exposed may be adjusted to provide a desired pore size, shape, and composition for application in lateral flow assay devices. Pore sizes in the strip may affect one or more performance characteristics such as protein binding capacity, speed of fluid transfer, and detection sensitivity. For example, larger pore sizes may allow faster fluid transfer which may reduce procedural time. However, larger pores may also decrease protein binding capacities of capture ligands, lowering the detection sensitivity. Alternatively, samples of higher viscosity may require larger pore size in order to migrate sufficiently. Further, the size and binding affinities of different biomolecules may be taken into consideration, as larger pore sizes may allow sufficient binding of high affinity molecules while also reducing procedural time. Therefore, a desired pore size may be determined based on the desired performance characteristic of the assay being produced. Further, various features may enable ease of development and production, eliminating time-consuming steps of cutting and fitting seen in current systems to manufacture porous film strips.

It is further understood that the lateral flow test device and method described and illustrated herein represents only example embodiments. It is appreciated by those skilled in the art that various changes and additions can be made to such device and method without departing from the spirit and scope of this application. For example, method 400 may comprise additional steps for optimizing pore size utilizing various dispensing head types, robotic set-ups, and selected combinations of solvents, non-solvents, nitrocellulose, hydrophilic additives, detergents, and meta-solvents. Moreover, materials aside from nitrocellulose may be used, such as polyamide-based membranes, glass fiber, cellulose, and other microporous polymers, singularly or in combination with other polymers, depending on compatibility with a variety of ligands and/or binding structures, such as biomolecules (e.g., proteins, antibodies, capture ligands) and nanoparticles (e.g., gold). The components, thickness and lengths of the polymeric strips may be chosen to adjust the wicking rates and therefore timing of the transportation of one or more of the compounds to be used in an assay formed using the polymeric strips.

The resulting polymeric strips may be used in a variety of applications including as part of a qualitative or quantitative assay of biomolecules. In some examples, analytes of interest may bind directly to the polymeric strips. In other examples, capture of the analytes of interest may be enhanced by first coupling the biomarker of interest in a sample to a protein with better solid phase-binding properties or by attaching binding ligands with better solid phase-binding properties to the polymeric strips prior to introducing the biological sample. Suitable coupling proteins include, but are not limited to, macromolecules such as serum albumins including bovine serum albumin (BSA), keyhole limpet hemocyanin, immunoglobulin molecules, thyroglobulin, ovalbumin, etc. Other reagents that can be used to bind molecules to the nitrocellulose matrix strip include polysaccharides, polylactic acids, polyglycolic acids, polymeric amino acids, amino acid copolymers, and the like. In some examples, the binding ligands may be selected such that cross-reactivity between the binding ligands and other biomolecules is minimized. That is, each binding ligand may specifically bind to/interact with one biomarker target and not biomarkers targeted by the other binding ligands and/or biomolecules irrelevant to disease diagnosis.

Such binding ligands and methods of coupling the binding ligands to antigens are well known to those of ordinary skill in the art. For example, in some embodiments, the nitrocellulose matrix strip may be extruded with binding ligands. In other embodiments, binding ligands may be printed on the nitrocellulose strip after it dries, for example in a microarray. Such binding ligands may be printed on one or more portions of the nitrocellulose strip, forming one or more analyte capture zones. Such capture zones may be used to capture the same or different analytes of interest which may comprise the same or different types of biomolecules including, but not limited to, antibodies, antigens, oligonucleotides, and RNA. The resulting bound analytes of interest may be imaged by an imaging device such as the device described in further detail in FIGS. 10-15.

After reacting the nitrocellulose matrix strip with binding ligands, any non-immobilized binding ligands may be removed from the nitrocellulose matrix strip by washing, and the nitrocellulose matrix strip-bound binding ligands are then contacted with a sample (such as a biological sample) that is to be assayed for the presence of a target analyte, such as a protein, carbohydrate, lipid, DNA, RNA, or other polynucleotide having a ligand moiety that can couple to the biomolecules immobilized on the solid support. After washing to remove any unbound target analytes, a secondary binder moiety that is detectably labeled may be added under suitable binding conditions, wherein the secondary binder is capable of associating selectively with the bound ligand. The presence of the labeled secondary binder moiety may then detected using, for example, a microarray assembly as described in more detail with reference to FIGS. 10-16. While any label may be used, in some examples, the labeled secondary binder moiety may include a fluorescent species, such as a fluorescent dye (e.g., an organic fluorophore) or a fluorescent semiconductor nanocrystal (QNC, also called a quantum dot), for fluorescence detection. In other examples, the labeled secondary binder moiety may include redox molecules, radiolabels, or beads for detection.

For example, the labeled secondary binder moiety may include fluorescent dye-based conjugates, wherein fluorescent dyes are conjugated to biomolecules such as antigens or antibodies (e.g., via bioconjugation). However, due to the lower efficiency of the available fluorophores, detection and imaging methods require scanners whose optical excitation source is very bright and detection methods that are extremely sensitive. The inventors herein have realized that by taking advantage of the resonant scattering of porous nitrocellulose (PNC) films, fluorescence amplification can be increased when PNC is used as the microarray substrate. This is because the fluorophore label will emit light not only when stimulated by photons that strike the label from the incoming direction of the excitation beam, but also from light reflected back by the PNC. As such, this increase can be realized for a variety of fluorescent species, including fluorophores and QNCs, and similar fluorescent labels whose extinction coefficient is at or above 10⁶ M⁻¹ cm⁻¹.

Target analytes that can be analyzed include biomolecules such as proteins, polypeptides, and single- or double-stranded nucleic acid molecules that contain a target nucleotide sequence. Protein and polypeptide analytes may include post-translational modifications of the polypeptide, for example, glycosylations, acetylations, phosphorylations, and the like. In addition, protein fragments, analogs, mutated or variant proteins, fusion proteins and the like are included within the meaning of polypeptide. Nucleic acid analytes may be from a variety of sources, e.g., biological fluids or solids, chromosomes, food stuffs, environmental materials, etc., and may be prepared for the hybridization analysis by a variety of means, e.g., proteinase K/SDS, chaotropic salts, or the like. Furthermore, nanocrystals may be conjugated to molecules that can interact physically with biological compounds such as cells, proteins, nucleic acids, subcellular organelles and other subcellular components. For example, nanocrystals can be associated with biotin, which can bind to the proteins avidin and streptavidin. The various analytes described above may be adapted to multiple types of array or other assays that may be imaged to detect one or more analytes of interest.

Using one or more microarrays of binding ligands, a single porous nitrocellulose strip may be used to test for a plurality of different biomarkers for a plurality of diseases and conditions. In some examples, a plurality of binding ligands may be included for each disease causing pathogen assessed by the microarray. For many infectious diseases, multiple antibody classes (e.g., IgG, IgM, IgA) and/or subclasses may be used for disease diagnosis. Further still, one or more pathogen-specific antigens may also be included. For example, IgG antibodies usually take several days to weeks after the appearance of disease systems (e.g., fever) to be detectable in peripheral blood, which limits the sensitivity of tests that probe only for IgG as compared with measurement of IgM antibodies that appear more rapidly. IgG specificity can also be altered by cross-reactivity with other infectious agents, background sero-prevalence in the healthy exposed population, and long-term persistence of antibodies from prior infections. Therefore, the simultaneous detection of IgM and IgG antibodies and the one or more pathogen-specific antigens may enable a positive diagnosis. While a single test region is shown, multiple microarray test regions with the same or different pre-fixed binding ligands may be located on a single porous nitrocellulose strip forming a plurality of analyte capture zones. The analyte capture zones may include one or more control regions. The one or more control regions may further include one or more positive controls and one or more negative controls, which serve to ensure the integrity of the biological sample as well as correct processing of the microarray.

Nitrocellulose strips may be pre-printed or dispensed directly onto a variety of solid substrates to be used in assays such as those described above. FIG. 5 shows an embodiment of the substrate 104 onto which a polymer mixture (e.g., mixture 106 shown above in FIGS. 1-3) may be dispensed directly upon in accordance with the method of FIG. 4. As shown in FIG. 5, the substrate 104 may be a flat substrate 500 comprising a barcode, QR code, or other type of label 510 for positive sample identification, and an optional opening 518 to assist in assembling the flat substrate 500 into a larger device, or for locking it in place in an imaging apparatus. In some examples, the flat substrate 500 may be part of a larger device such as the cartridge shown in FIGS. 8A-8C, or may be used independently, moving the substrate 500 from reagent to reagent in a pre-identified sequence. A portion or all of the flat substrate 500 may be coated with a nitrocellulose film 107, the nitrocellulose film 107 including a plurality of analyte capture locations or wells 516. The nitrocellulose film may be produced according to the method of FIG. 4 and printed with capture ligands 524 in the analyte capture locations 516 according to the example method of FIG. 9. The microarray 515 with capture ligands 524 may chemically bind biomolecules of the sample (e.g., biomarkers) to the flat substrate 500. In the description herein, the capture ligands 524 in the capture locations 516 may also be referred to as spots 524. In some examples, the flat substrate 500 as described above may further comprise a sample application area, conjugate pad or absorbent pad as described in more detail with reference to FIGS. 6, 7, and 8A.

FIG. 6 shows a side view embodiment of a nitrocellulose matrix strip 600 as part of a LFA. Thus, matrix strip 600 may be the same or similar to strip 107 described above with reference to FIGS. 1-2 and 5. Strip 600 is generally a flat, elongated and rectangular piece comprising three regions 604, 608, and 612 comprising a polymer mixture including nitrocellulose disposed directly on the housing substrate 602. Housing substrate 602 may be the same as or similar to substrate 104 described above with reference to FIGS. 1-2 and 5.

First region 604 may be an area wherein a sample is loaded and received. In one example, the sample application first region 604 may pretreat the sample prior to transportation, for example it may separate sample components, remove interferences, adjust the pH, or the like. More specifically, a collection window configured to direct a fluid of interest to the first region 604 may be disposed directly above first region 604. In one embodiment, first region 604 may be provided with a fibrous layer 606 deposited over the nitrocellulose matrix strip 600 that facilitates capillary action to distribute sample fluid to the downstream regions 608 and 612. Specifically, the layer 606 may be disposed on a top surface 622 of first region 604, the top surface 622 opposite a bottom surface 624, where the bottom surface 624 may be in physical contract with the substrate 602 and/or a base piece 620 which may be the same as or similar to mask 112 described above with reference to FIG. 2. Thus, the top surface 622 may face away from the substrate 602, and the first region 604 may be positioned between the substrate 602 and the layer 606. In one example, the fluid of interest may be applied directly to first region 604. In another example, the fluid of interest, such as a bodily fluid, may undergo processing and/or treatment, such as by adding one or more reagents, before being applied to first region 604. In a further example, dried reagents may be present on the polymeric strip. Such reagents may be reconstituted through the addition of the bodily fluid, the addition of a buffer, or any other method generally used to reconstitute dried reagents.

Adjacent to and downstream of first region 604 is a first partition 616, wherein approximately no ligands, proteins, antibodies or other biomolecules may be loaded and impregnated into strip 600. Thus, substantially no binding and/or detection may occur between analytes in the sample and first partition 616. The first partition 616 may be sized to approximately the same width as analyte capture zone 608. Said another way, first partition 616 may be raised from the surface of the substrate 602 by an amount approximately equal to that of the analyte capture zone 608.

Adjacent to first partition 616 on the opposite side from first region 604 is a first analyte capture zone 608. In one example, analyte capture zone 608 may be the same or similar to the microarray of binding regions discussed previously in reference to FIG. 5, wherein one or more known binding partners or ligands of a desired biomolecule of interest, such as a protein or antibody, are impregnated into the strip 600. In one example, to form this region, a solution containing a plurality of binding ligands is dispensed and loaded onto first analyte capture zone 608. Through capillary action, the loaded binding ligands disperse within the nitrocellulose matrix and are stably fixed and integrated within the matrix. In another example, the plurality of binding ligands may be printed onto the first analyte capture zone 608 in a defined geometry, such as according to the method of FIG. 9. After fixation of binding ligands to the analyte capture zone 608 of the strip, a blocking solution may be applied to the strip 600. The blocking solution may reduce and/or prevent immobilization of biomolecules such as proteins or antibodies in free sites on the porous strip 600. Blocking solutions may include, but are not limited to, one or more of protein blocking solutions and/or non-protein polymer blocking solutions. Therefore, interaction, binding, or crosslinking of one or more biomolecules in the unknown sample to the fixed ligands in the strip 600 may occur as the sample fluid moves through analyte capture zone 608 from sample collection region 604 and first partition 616. Thus, the sample fluid may disperse across the strip 600 from left to right in FIG. 6, as shown by flow arrow 626.

The analyte(s) of interest may be labeled in a variety of ways. In one example, labels such as flurophores or other fluorescent labels may be used. In another example, first analyte capture zone 608 may include a chromogenic substrate, which may recognize and enzymatically react with the biomolecule of interest in the sample, or crosslinking or binding of the biomolecule of interest and the integrated ligand, to produce a visible color. In another example, a fluorogenic substrate may be used, the fluorogenic substrate reacting with the biomolecule of interest in the sample to produce fluorescence. The fluorogenic substrate is a non- or weakly fluorescent species that is transformed into a detectibly fluorescent species upon the specific interaction with the biomolecule of interest. The chromogenic or fluorogenic substrate may be applied to nitrocellulose strip 600 at analyte capture zone 608 by mixing it with the solution of the binding ligands or may be dispensed separately in another step. For example, the chromogenic or fluorogenic labels may be located on a conjugate pad over which the sample will flow.

The visible color of the chromogenic substrate may be viewed through a detection window to determine whether a biomolecule of interest is present in the unknown sample. Similarly, the fluorescence emission of the fluorogenic substrate may be measured through the detection window via a fluorescence imager to determine whether the biomolecule of interest is present in the unknown sample. The chromogenic or fluorogenic reaction may additionally be viewed and analyzed using a biomolecule analysis system, as further described with respect to FIGS. 10-16.

Downstream and adjacent to the analyte capture zone 608 is a non-overlapping second partition 618. In some examples, the size and length of second partition 618 may be comparable to first partition 616. In other examples, second partition 618 may be larger and longer than first partition 616. Thus, the first partition 616, analyte capture zone 608, and second partition 618 may be approximately flush with one another. Adjacent to and sequentially downstream of second partition 618 is a second analyte capture zone 612. In one example, the second capture zone 612 may be a control region comprising one or more known binding partners or ligands to a protein that is generally considered to be present in a sample fluid. In one embodiment, the ligands loaded into analyte capture zone 608 may be substantially dissimilar to the ligands loaded into second analyte capture zone 612 in structure and/or function. Thus, analyte capture zone 608 may serve as a control to ensure the integrity of biomolecular structures in the sample and strip 600 as well as the functionality of the LFA test. Similar to the first analyte capture zone 608, a chromogenic or fluorogenic substrate or label may also be included in the second analyte capture zone 612. In some embodiments, the chromogenic or fluorogenic substrate or label may be the same or different than the substrate used in the first analyte capture zone 608. The visible color and/or fluorescence emission of the chromogenic or fluorogenic substrate may be viewed and/or imaged through the detection window to determine if the biomolecule of interest is present in the unknown sample. The chromogenic or fluorogenic reaction may additionally be viewed and analyzed using a biomolecule analysis system, as further described with respect to FIGS. 10-16.

An additional handling region may be included at either end of the strip, wherein a user can handle and maneuver the nitrocellulose strip without contaminating sensitive wicking and detection regions (not shown).

In addition, FIG. 6 shows an example of a nitrocellulose strip 650 including a base piece 620 disposed between the substrate 602 and the strip 650. In one embodiment, strip 650 is identical to strip 600. Thus, components of the strip 650 may be the same or similar to strip 600. As such, components of strip 650 numbered the same as components of strip 600 already described herein may not be reintroduced or described again. In the example of strip 650, the strip 600 may be dispensed onto the base piece 620, which may be disposed on the substrate 602. Thus, the strip 600 may not be disposed directly on the substrate 602. Base piece 620 may be the same or similar to mask 112 described above with reference to FIG. 2. Thus, base piece 620 may be disposed directly atop housing substrate 602. In one example, base piece 620 is formed from a polymer mixture and dispensed by a dispensing device (e.g., dispensing device 102 shown in FIGS. 1-2) onto substrate 602. After polymerization and drying, strip 650 may be dispensed and formed onto the base piece 620. In this way, base piece 620 may provide increased support for the nitrocellulose strip 650 as compared to examples where the base piece 620 is not included, such as in the example shown for strip 600.

In some examples, the locations of the various detection regions or capture zones on the strips 600 and 650 may vary. For example, analyte capture zones 608 and 612 may be switched such that the control region is upstream of the test region. In yet other embodiments, various detection regions may fully or partially overlap each other or may comprise separate, non-overlapping regions (such as those shown in FIG. 6). Similarly, the sample application first region 604 may overlap the regions impregnated with the detection ligands or may be a separate region. It may be appreciated that the specific locations of the various regions on strips 600 and 650 may vary depending on desired outcomes.

In other examples, first analyte capture zone 608 and/or second analyte capture zone 612 may comprise one or more microarrays of biomolecules that serve as binding ligands to target analytes. The biomolecules may be printed onto the nitrocellulose matrix strip in a grid or other defined geometric arrangement via a robotic system, as further described with respect to FIG. 9.

FIG. 7 shows an embodiment of the substrate 104 that a polymer mixture (e.g., mixture 106 shown above in FIGS. 1-3 and described in FIG. 4) may be dispensed directly upon. In one embodiment, the substrate 104 may be an elongated device housing comprising a top member 700 and bottom member 720, wherein members 700 and 720 can be snapped reversibly but securely together via engagement of complementary male and female parts: primary pins 708a-708f and holes 726a-726f on the top and bottom members 700 and 720, respectively. Specifically, the primary pins 708a-708f may be positioned on an interior facing first surface 702 of the top member 700. Thus, the pins 708a-708f may be physically coupled to the first surface 702 and may protrude from the first surface 702. The first surface 702 may be opposite an exterior-facing second surface 703 of the top member 700. First surface 702 may be relatively flat and/or planar.

The holes 726a-726f may be positioned on an interior-facing first surface 722 of the bottom member 720. First surface 722 may be relatively flat and planar. Thus, the holes 726a-726f may be physically coupled to the first surface 722, and may protrude from the first surface 722. Specifically, the holes 726a-726f may protrude from the first surface 722 and may each include an opening sized to receive the pins 708a-708f. Although six pins and six holes are shown in the example of FIG. 7, it is important to note that in other examples, fewer or greater than six pins and/or holes may be used to physically couple the top member 700 and bottom member 720.

As such, when coupling the top member 700 and bottom member 720 to one another, the top member 700 and bottom member 720 may be oriented so that the interior-facing first surfaces 702 and 722, respectively, are facing one another. Thus, the top member 700 may be flipped 180 degrees from the orientation shown in FIG. 7 so that the pins 708a-708f are pointed towards and/or facing the holes 726a-726f. More specifically, the top member 700 may be rotated around the central axis X-X′ by approximately 180 degrees from the orientation shown in FIG. 7.

In other words, each of the pins 708a-708f may fit into one of the respective holes 726a-726f formed along the perimeter of the interior facing surfaces 702 and 722 of the top and bottom members 700 and 720, respectively. In this way, the top member 700 and bottom member 720 may be physically coupled to one another, by inserting the pins 708a-708f into the holes 728a-728f As such, when the pins 708a-708f are inserted into the holes 728a-728f and the top member 700 and bottom member 720 are physically coupled to one another, relative movement between the top member 700 and bottom member 720 may be restricted and/or inhibited.

A lip 704 may extend from first surface 702 of the top member 700 around a perimeter of the first surface 702. The lip 704 may be raised from the first surface 702. Similarly, a lip 724 may be included on the first surface 722 of the bottom member 720 around a perimeter of the first surface 722. The lip 724 may be raised from the first surface 722. When the top member 700 and bottom member 720 are coupled to one another, there may be constant and contiguous physical contact between the lips 704 and 724 of the top and bottom members 700 and 720, respectively.

The top member 700 may include an exterior-facing second surface 703, opposite the interior-facing first surface 702. Similarly, the bottom member 700 may include an exterior-facing second surface 723, opposite the interior-facing first surface 722. Thus, when the top and bottom members 700 and 720, respectively, are physically coupled to one another to form the substrate 104, the interior-facing first surfaces 702 and 722 may not be visible when viewing the substrate from exterior to the substrate 104. However, the exterior-facing second surfaces 703 and 723 may be visible when the members 700 and 720 are physically coupled to one another. Second surfaces 703 may in some examples be relatively flat and/or planar surfaces.

In one embodiment, each of the top and bottom members 700 and 720 may be generally rectangular in shape and may be made from plastic or another appropriate material. The plastic of the substrate may be clear or opaque. The particular shape and construction of the top and bottom members 700 and 720 included in substrate 104 may be varied from the example illustrated, if desired.

Two examples of the bottom member 720 are shown in FIG. 7. In a first example, shown in FIG. 7 positioned above a second example, the mixture has not been dispensed onto the member 720. In the second example of the bottom member 720, shown in FIG. 7 below the first example, the mixture has been dispensed onto the bottom member 720 to form the nitrocellulose matrix strip 107. Thus, the first example shows the bottom member 720 prior to fabrication of the strip 107, such as in step 1 shown above with reference to FIGS. 1 and 2. The second example shows the bottom member 720 after fabrication of the strip 107, where the mixture has been dispensed, polymerized, and dried to form the strip 107 on the bottom member 720.

In some examples, the bottom member 720 may include an area therein to receive the dispensed polymer mixture along the bottom member's longitudinal axis. In one embodiment, a region 730 may be the area wherein the polymer mixture will be dispensed. Thus, region 730 may be the same or similar to well 114 described above with reference to FIG. 2, and as such, may be a recess within the first surface 722 of the bottom member 720 for receiving and retaining the mixture. As such, the region 730 may serve to one or more of shape, retain, and/or form the strip 107. In other examples, the shape and size of region 730 may be an alternate configuration.

Once dried and polymerized under a set of specific conditions as previously described, the resulting strip 107 may include, but is not limited to: a collection region 731, a first and second analyte capture zone 732 and 734, respectively, and a handling region 736. In one example, collection region 731 may provide wicking action to facilitate capillary action of a fluid to analyte capture zones 732 and 734. Moreover, the first analyte capture zone 732 may be a test region (denoted in this example by the letter “T”) wherein one or more proteins of interest in an unknown sample fluid may bind to one or more pre-fixed and known binding ligands, such as a protein or antibody. In some examples, the pre-fixed and known binding ligands may be printed in a microarray, allowing for the capture of one or more groups of biomarkers of interest. In some examples, the binding ligands may correspond to targets for detecting a plurality of infectious and non-infectious diseases. In other examples, the binding ligands may correspond to targets for detecting a plurality of environmental contaminants. In one example, the second analyte capture zone 734 (denoted in this example as the letter “C”) may be a control region comprising one or more pre-fixed and known binding ligands considered to be present in a sample fluid. Thus, this region serves as a control to ensure the integrity of biomolecular structures in the sample, as well as functionality of the LFA test. In some examples, the binding ligands may be printed onto the first analyte capture zone and the second analyte capture zone 734 in a mircoarray, as further described below with respect to FIG. 9. A handling region 736 may be included on the strip 107 to enable a user to handle and maneuver the strip 107 without contaminating the analyte capture zones 732 and 734. The aforementioned descriptions of each region and configurations of a strip of polymer are one example, and may be modified, if desired.

Furthermore, the top member 700 may include one or more windows, such as windows 714 and 716. In one embodiment, window 716 may be an opening through which the polymer mixture and/or strip 107 may be observed after the mixture has been dispensed onto the bottom member 720 by a dispensing device (e.g., dispensing device 102 shown in FIGS. 1-2) and the top and bottom members 700 and 720, respectively are physically coupled together. Thus, the window 716 may be a hollow opening. The window 716 may be optically clear, so that light reflected from the strip 107, may pass in a relatively unobstructed manner through the window 716, and out of the substrate 104.

In one embodiment, the first surface 702 of the top member 700 and/or the first surface 722 of the bottom member 720 may include one or more secondary pins for retaining and holding the strip 107 in place. For example, as shown in FIG. 7, secondary pins 710a-710c may be physically coupled to the first surface 702 of the top member 700, such that the secondary pins 710a-710c extend inwards towards the bottom member 720. In some examples the secondary pins 710a-710c may be referred to as holding elements since they may serve to hold the strip 107 in place. Thus, the secondary pins 710a-710c may protrude from the first surface 702 and may physically contact edges of the strip 107 so that movement of the strip 107 relative to the bottom member 720 is restricted. Said another way, the secondary pins 710a-710c may be positioned around a circumference of the strip 107. Additionally or alternatively, there may be pins on the first surface 722 of the bottom member 720 that restrict movement of the strip 107 relative to the bottom member 720.

The top member 700 may also include a second collection window 714, including a funnel 712, so that the collection of a fluid of interest may be funneled through the window 714 to be absorbed by region 731 of a strip 107. Thus, after the strip 107 has been formed and the top and bottom members 700 and 720 have been physically coupled to one another, the fluid of interest may be poured/dispensed onto the region 731 via the window 714. Thus, the fluid of interest may first enter the substrate 104 via the window 714 of the top member 700. As such, the collection window 714 may be positioned directly vertically above the region 531 so that fluid entering the substrate 104 via the window 714 collects in the region 731.

Adjacent to the first window 716 may be denotations of one or more analyte capture zones, such as test analyte capture zone 732 and control capture zone 734. For example, in one embodiment, a letter “C” may be printed on the surface of top member 700 directly vertically above control capture zone 734 if viewed through the opening to the strip underneath the top member 700. Similarly, in another example, a letter “T” may be printed in a similar fashion directly above the test analyte capture zone 732. Thus, the letters may be printed onto the first window 716 to indicate which portion of the strip 107 is being viewed underneath the top member 700. Any combination of symbols may be printed on either member to denote various features. Thus, the window 716 may allow a user to view the test analyte capture zone 732 and control capture zone 734 from exterior the substrate after the fluid of interest has been dispensed on the strip 107.

Turning to FIGS. 8A-8D, FIGS. 8A and 8B show an embodiment of a cartridge enclosing the substrate 104 onto which a polymer mixture (e.g., mixture 106 shown above in FIGS. 1-3) may be dispensed directly upon in accordance with the method of FIG. 4. While the substrate 104 is shown as a rectangle, it may be any size or shape desired. In some examples, it may be square. In other examples, it may be round, in further examples it may be an oval. FIG. 8A shows an exploded view of the cartridge 800. FIGS. 8B and 8C show a top angled view of various embodiments of the cartridge 800, and FIG. 8D shows an alternate fluidic gasket that may be used in the cartridge. The cartridge 800 may be comprised of a housing, including an upper first portion 802 and a housing base 804 with a housing base bottom 824, a fluidic gasket 816 that includes microfluidic channels and chambers, a sealed liquid reagent pack 814, and a waste collection material 820. Thus, the cartridge 800 may be a fluidic “lab-on-a-chip” with lateral flow reagent transport. Furthermore, in some examples, the cartridge 800 may be disposable, such as intended for a single use.

The housing and the fluidic gasket 816 may be comprised of plastic, including polycarbonate, acrylic, and cyclic olefin copolymer, and/or another suitable material. In some examples, the material is such that the labels attached to molecules bound to a microarray may be read through the plastic, i.e. the plastic is optically transparent to the wavelengths used by the reader. In other examples, the housing may include a window such as window 812 through which the labels may be viewed. For example, the upper first portion 802 and the fluidic gasket 816 may include a barcode window 812, through which a barcode, QR code, alphanumeric code or other identifier 810 printed on the substrate 104 may be read. In some aspects, the identifier 810 may be computer readable. In other aspects it may be readable to a human. In further aspects, it may contain information all or part of which may be ready by a computer or a person. In some aspects, the identifier 810 may contain information related to the manufacturing of the substrate 104 and/or unique features that may be used to track an individual sample identification. The cartridge 800 may further include a microarray window 811 through which colorimetric or fluorescent reactions taking place on the microarray may be viewed or imaged. In one embodiment, windows 811 and 812 may be an opening through which the polymer mixture and/or strip 107 may be observed after the mixture has been dispensed onto the substrate 104 by a dispensing device (e.g., dispensing device 102 shown in FIGS. 1-2) and the top and bottom members 802 and 804, respectively have been physically coupled together. Thus, the windows 811 and 812 may be hollow openings. In other aspects, the barcode and/or microarray may be covered by a transparent substance (not shown) protecting the microarray and/or barcode from contamination. The windows 811 and 812 may be optically clear, so that light reflected from the strip 107, may pass in a relatively unobstructed manner through the window 812, and out of the substrate 104. In additional examples, the substrate 104 may be removable from the cartridge, such as a cartridge with or without windows such that once the analytes of interest are bound, the substrate 104 including the microarray and identifier may be imaged independently of the cartridge as shown in more detail in FIGS. 10-14.

The upper first portion 802 further includes a sample port cover 803, which removably covers a sample port 808. A biological sample, such as blood from a patent finger stick, may be applied to the sample port 808. In some examples, the sample port may have a size that allows a predetermined volume of sample to be collected. The fluidic gasket 816 fluidically couples the sample port 808 to the substrate 104 and the nitrocellulose film 107 coated on the substrate 104. In some examples, a particulate filter 809 may be attached to the sample port 808, preventing particulates from being flowed into a sample well and interfering with analyte labeling and binding of analyte-label conjugates to the microarray in the microarray incubation chamber 818. In some examples, as shown in FIG. 8D, a plurality of channels 821a, 821, b and 821c may be used to guide the liquid to pool over the nitrocellulose film 107 in the microarray incubation chamber 818. Such channels may be used to direct one or more reagents along one or more pathways at the same or different times. For example, as shown in FIG. 8D, there are two channels 821b and 821c, of different lengths and shapes. The additional length provided by 821c delays the transport of fluid from a reservoir housed in a container placed in opening or reagent reservoir 813b, allowing the reactions initiated by the reagents housed in a container in 813a to occur at an earlier time point than the reactions initiated by the reagents housed in 813b. As described above with respect to FIG. 5, the nitrocellulose film 107 may include a plurality of analyte capture locations or wells, which may be printed with capture ligands according to the example method of FIG. 9 to produce a microarray 815. The microarray 815 with capture ligands may chemically bind biomolecules of the sample (e.g., biomarkers) to the substrate.

The upper first portion 802 may further include a reagent pack reservoir housing 806. The reagent pack reservoir housing 806 may house a sealed liquid reagent pack 814 which may sit in a reagent reservoir 813 in the fluidic gasket 816. For example, the sealed liquid reagent pack 814 may be a blister pack or bubble pack. The sealed liquid reagent pack 814 stores liquid reagents, which may include one or more aqueous buffers (e.g., sterile phosphate buffered saline, or PBS) in sufficient volume to solubilize dried (e.g., lyophilized) probe molecules (e.g., fluorescently labeled antibodies) in an adjacent chamber or well 817 as well as dilute the biological sample provided via the sample port 808 to sample well 819, solubilize dried binding ligands and/or wash the microarray 815 accessible through the microarray incubation chamber 818. Excess fluid may then drain through waste collection area located in the base of the cartridge. Washing the microarray 815 may reduce a non-specific background signal when imaging the microarray, for example.

In some examples, the sealed liquid reagent pack may comprise a series of layers actuated by varying degrees of force, allowing different reagents to be released in a sequential manner from the same sealed liquid reagent pack. In other examples, the fluidic gasket 816 may comprise a series of openings 813 such as 813a and 813b shown in FIG. 8D which may be used to house a plurality of a sealed liquid reagent packs containing the same or different reagents. The reagents may flow down the same or different channels 821 to reach the microarray 815. For example, as shown in FIG. 8D, reagents in a sealed liquid reagent pack housed in 813a and 813b may be actuated simultaneously or sequentially. The reagents housed in the sealed liquid reagent pack in the opening or reagent reservoir 813a flow through a channel 821b to interact with dried probe molecules in the adjacent chamber 817, hydrating them and bringing them in contact with the sample in sample well 819 and then to the microarray 815 in a microarray incubation chamber 818. The microarray 815 may then be washed by a reagent released from a sealed liquid reagent pack housed in opening 813b of the fluidic gasket 816 and released through channel 821c which is constructed to delay the arrival of the wash at the microarray incubation chamber until after the molecular probes and analytes of interest in the sample have reacted with the microarray in the microarray incubation chamber. The delay in the arrival of the wash can be engineered by any means generally used. In some examples, as shown in FIG. 8D, the delay may be engineered by creating a winding channel 821c with a plurality of turns, including 3, 4, 5, 6, 7, 8, 9, 10 or more turns delaying the arrival of the wash until sufficient time has passed for the analyte of interest to be bound with a label as well as being bound to binding ligands in the microarray. Washing the microarray 815 may reduce a non-specific background signal when imaging the microarray, for example. In some examples, the various compartments represented by the fluidic gasket may form part of the substrate, interior of the housing or other structure, and thus perform the same function as the fluidic gasket without requiring the fluidic gasket.

The sealed liquid reagent pack may be actuated using any means generally used. In some examples, it may be actuated using pressure or a puncture mechanism. In some examples, the liquid reagent pack may comprise a plurality of materials which require the same or different amounts of force to rupture, allowing for different reagents to be held in isolation in the same liquid reagent pack, e.g. in a compartmentalized or layered manner. As an example, the sealed liquid reagent pack 814 may have a reagent volume that is at least ten times a void volume of the microarray incubation chamber 818 which may also referred to as a reaction chamber. Furthermore, the one or more aqueous buffers may be non-precipitating at cold temperatures (e.g., 4° C. and lower), enabling the cartridge 800 to be stored in a variety of conditions.

The sealed liquid reagent pack 814 may be situated such that it is surrounded by each layer in the cartridge. For example, it may be covered by a reagent reservoir housing 806 in the upper first portion 802. Such a housing may be raised as shown in FIG. 8A, flat, or depressed. The liquid reagent pack 814 may sit in the fluidic gasket 816 such that when it is actuated, the liquid reagents flow through at least one of channels 821a or 821b to come in contact with the lyophilized conjugate labels in 817 and the sample in the sample well in 819, before coming into contact with the microarray 815 in the microarray incubation chamber 818. The liquid reagent pack may further sit in an opening 807 in the substrate 104 such that the liquid reagent pack 814 is accessible though opening 805 in the housing base 804. A technician may access the sealed liquid reagent pack 814 through the opening 805 to break open the sealed liquid reagent pack 814 to initiate flow of the liquid reagents through the microfluidic channels and chambers of the fluidic gasket 816. For example, deployment of the reagents from the sealed liquid reagent pack 814 into the fluidic channels/chambers of the fluidic gasket 816 may be performed by a single press of a finger. After flowing through the reaction chamber 818, liquid may flow through waste channel ports 822 in the substrate and the housing base 804 to the waste collection material 820, which may be an absorbent pad, for example. The waste collection material 820 may be enclosed in the housing base 804 through the placement of housing base bottom 824 which may partially or entirely cover the undersurface of housing base 804 and may be permanently or removably affixed. In some embodiments, the housing base bottom 824 may slide or otherwise provide access to actuate liquid reagent pack 814.

FIG. 9 shows an example method 900 for producing a microarray for disease diagnostics. For example, the microarray may include a plurality of immobilized binding ligands that are printed onto a nitrocellulose film coated on a substrate. While any shape substrate may be used, in some examples the substrate may be a flat substrate to enable ease of handling and microarray processing, as further described in FIGS. 10-14 and 16, below. The immobilized binding ligands may be antibodies or antigens for identifying biomarkers in a biological sample (e.g., from a patient or the environment). Multiple diseases/conditions/contaminants may be assessed using a single microarray, including infectious and non-infectious diseases. Thus, the patient may be diagnosed with one or more diseases or conditions based on the presence of one or more biomarkers, as detected by processing the microarray with the biological sample and by imaging the microarray with an imaging assembly. In particular, an open system nature of the microarray design allows for unlimited pathogen differentiation combinations by simply printing different layouts of antigens and antibodies.

Method 900 begins at 902 and includes forming the nitrocellulose film on the substrate. For example, a nitrocellulose mixture (e.g., mixture 106 shown in FIGS. 1-3) may be deposited onto the substrate by a dispensing device (e.g., dispensing device 102 shown in FIGS. 1-2) in a defined area, such as according to the example method 400 of FIG. 4. The substrate may be relatively planar and may include a flat surface upon which the nitrocellulose mixture is deposited. For example, the substrate may represent substrate 104 shown in FIGS. 1-3 and 5-7, and, once dried, the nitrocellulose mixture may form a film having a desired pore density and/or pore size as well as a desired geometry. For example, the nitrocellulose film may represent strip 107 shown in FIGS. 1-2, 5 and 7. The nitrocellulose film may be sized to approximately a 12 mm by 12 mm square. However, in other examples the film may be sized to be larger or smaller than 12 mm by 12 mm. Further, in other examples, the film may be shaped differently than a square, such as circular, triangular, rectangular, S-shaped, etc. For example, the nitrocellulose film may be a 7.5 mm by 10 mm rectangle.

At 904, method 900 includes printing the microarray onto the nitrocellulose film. The microarray includes a plurality of binding locations, also known as analyte capture locations, creating an analyte capture region, each of the analyte capture regions including one or more binding ligands targeting a biomarker of interest. In examples where the microarray is configured as a protein microarray, the analyte capture regions may include purified antigens to which antibodies in the biological sample, serving as biomarkers, may bind. Thus, binding ligands, for example antigens, may be printed (e.g., spotted) onto the nitrocellulose film at the analyte capture regions. However, it should be appreciated that in other examples, the analyte capture regions may include molecules other than antigens for binding to biomarkers of the sample other than antibodies. For example, the binding ligands in the analyte capture regions may include antibodies, antibody fragments, or affibody molecules to which analytes of interest in the biological sample may bind. Nitrocellulose as a substrate has an advantage of very high and non-specific protein capture properties, so essentially all of the material applied is bound to the nitrocellulose. This ensures that the protein concentration per spot is consistent. The amount of protein printed on the nitrocellulose strip may be printed in any concentration desired. In some examples, the proteins may be printed at concentrations of 0.0001 to 0.5 mg/ml or any fraction thereof such as 0.3, 0.1, 0.03, 0.01, 0.003, 0.001 mg/ml (˜1 nl per spot). In still further examples, the analyte capture regions may include oligonucleotides or cDNA for binding to DNA or RNA in the sample. Further still, in some examples, fiducials may be printed onto the nitrocellulose film to serve as a point of reference during imaging, as will be further described with respect to FIG. 16. Further details of how the microarray may be printed and validated are described in Example 1, below.

The binding or analyte capture locations in the analyte capture regions may be printed onto the nitrocellulose film in a defined geometry, such as via a robotic system. Each of the binding locations may be approximately circular, and the diameter of each of the binding locations may be in a range between 100-1000 microns. Further, each of the binding locations may be relatively evenly spaced from one another, where the spacing between each of the binding locations may be in a range between 200 and 2000 microns. In some examples, the spacing of the binding locations may be based on the size of the binding locations, where the spacing between binding locations may be approximately twice the diameter of the binding locations. However, in other examples, the spacing may be greater than or less than two times the diameter of the binding locations. The nitrocellulose film may be configured to include approximately 1600 binding locations in one or more analyte capture regions. However, in other examples, the nitrocellulose film may include more or less than 1600 binding locations. For example, the nitrocellulose film may have a capacity of 300-400 target antigens and/or antibodies.

In additional examples, a microarray may comprise binding ligands targeting biomarkers or a series of biomarkers for a plurality of diseases with the same or similar symptoms, allowing a single test with a single sample to distinguish between diseases or conditions with similar appearances but distinct origins. For example, febrile illnesses may be caused by multiple types of bacteria, including gram positive bacteria (e.g., Streptococcus pneumoniae), gram negative bacteria (e.g., Salmonella typhi, spp.), spirochetes (e.g., Borrelia burgdorferi), intracellular bacteria (e.g., Mycoplasma pneumoniae), and mycobacteria (e.g., Mycobacterium tuberculosis); viruses (e.g., influenza virus A, B, or C); protozoa (e.g., Plasmodium falciparum or Plasmodium vivax); or nematodes (e.g., Schistosoma mansoni). In one example, a microarray may comprise binding ligands targeting biomarkers for the bacteria, viruses, protozoa, or nematodes that cause febrile illnesses, allowing the specific cause to be identified and an appropriate course of treatment designed.

For many infectious diseases, multiple antibody classes (e.g., IgG, IgM, IgA), antibody subclasses, and/or antigens may be used individually or in combination for disease diagnosis. Thus, more than one binding ligand for one or more types of biomolecules may be included for each disease. For example, a microarray may detect the presence of IgM and IgG antibodies as well as pathogen specific antigens and the combined information may be used to form a diagnosis. In other aspects, only one type of biomolecule such as an antibody or antigen may be measured for a certain disease. For example, in order to diagnose Plasmodium falciparum and Plasmodium vivax, generally only antigens are measured. For Dengue, both antigens and IgM are measured. For S. Typhi/Paratypha A, Leptospira spp, Orientia tsutsugamushi and Chikugunya virus, only IgM antibodies are measured. Exemplary microarrays could therefore bind Plasmodium falciparum and Plasmodium virax antigens, Dengue antigens and antibodies, and S. Typhi/Paratypha A, Leptospira spp, Orientia tsutsugamushi and Chikungunya virus antibodies, allowing a single microarray or group of microarrays on a single substrate to distinguish between a plurality of diseases with similar symptomology but distinct origins through the identification of the presence or absence of a plurality of biomolecules of different sizes and types.

In some examples, each binding ligand may be printed on the microarray in replicate, such as in duplicate or triplicate. Further still, the analyte capture regions may include one or more control locations. The one or more control locations may further include one or more positive controls and/or one or more negative controls, which serve to ensure the integrity of the biological sample as well as correct processing of the microarray. For example, purified human IgG and IgA may be included in the microarray separately and/or as mixtures and in serial dilution (e.g., a series of six two-fold dilutions starting with 0.5 mg/mL) as positive controls. Human IgA, print buffer only, and/or PBS only may be included on the microarray as negative controls to determine washing efficiency, non-specific binding, and background noise. Therefore, if there are 1600 analyte capture locations, the analyte capture locations may be targeted to biomarkers for fewer than 1600 diseases or conditions.

As an example, the microarray may enable detection and differentiation of more than 20 disease pathogens that cause a similar disease symptom (e.g., fever) from a single biological sample. As another example, the microarray may enable detection and differentiation of more than 30 disease pathogens that cause a similar disease symptom from a single biological sample. As still another example, the microarray may enable detection and differentiation of more than 40 disease pathogens that cause a similar disease symptom from a single biological sample.

The robotic system may spot a predetermined amount of each binding ligand in a predetermined pattern such that a location of each binding ligand within the microarray is known. In some examples, the pattern may be such that binding ligands targeting biomarkers for a same disease are grouped. For example, adjacent spots may contain binding ligands targeting biomarkers for the same disease diagnosis. In other examples, the pattern may be such that binding ligands targeting biomarkers for the same disease diagnosis are spaced apart (e.g., not located in adjacent spots) to increase signal clarity. In still other examples, a single spot may include a plurality of different binding ligands targeting biomarkers for the same disease diagnosis. For example, the single spot may include antigens for multiple classes and/or subclasses of antibodies that would be produced by the patient if infected with the disease-producing pathogen.

In some examples, the binding ligands and/or the placement of the binding ligands may be selected such that cross-reactivity between the binding ligands and other biomolecules is minimized. That is, each binding ligand may specifically bind to/interact with one biomarker target and not biomarkers targeted by the other binding ligand and/or biomolecules irrelevant to the disease/condition diagnosis.

In some examples, once printed, microarrays may be pre-blocked with a blocking solution, air-dried, and stored at room temperature with desiccant until used for testing. For example, a first portion of method 900, which refers to manufacturing the microarray (e.g., 902-904), may be performed at a first time, and a second portion of method 900 (e.g., 906-914), which refers to performing an assay with the microarray, may be performed at a second time. The second time may be a duration after the first time, such as days, weeks, months, or years. As such, an expiration date may be associated with the printed microarray to ensure that the microarray is used within its shelf life.

At 906, method 900 includes adding the biological sample to the microarray. For example, the biological sample may be or may be derived from saliva, blood (e.g., whole blood or plasma), urine, exhaled breath condensate, cerebrospinal fluid, sputum, tears, or other bodily fluids that include biomarkers of interest for infectious or non-infectious disease diagnosis. The sample may also be an environmental sample such as water, food, or plant and animal exudate which may include biomarkers for contamination or spoilage. In some aspects, the sample may be diluted with a buffer or detergent prior to adding it to the microarray. In further aspects, the sample may be diluted with a blocking buffer.

Upon adding the biological sample to the microarray, the binding ligands immobilized at the analyte capture regions may bind/immobilize corresponding biomarkers to the analyte capture locations. As an example, a portion of the substrate that includes the microarray may be dipped into a container or well holding the biological sample such that the entire microarray is submerged in the biological sample. In some examples, the microarray may remain submerged in the biological sample for a duration (e.g., 5-30 minutes) to facilitate binding of the biomarkers to the binding ligands. In other examples, the biological sample may be applied dropwise or with a swab to the microarray. When included in a cartridge, such as cartridge 800 shown in FIGS. 8A and 8B, the biological sample may be added via a sample port (e.g., sample port 808). Further, in some examples, the microarray may be incubated with the biological sample at a desired temperature and/or with shaking to further facilitate binding of the biomarkers to the binding ligands. Incubation may take place at any temperature conductive to facilitating binding.

In some examples, the microarray may then be washed with an appropriate solution, such as an aqueous buffer, to remove unbound biomolecules. The solution may contain a detergent to remove non-specifically bound biomolecules from the nitrocellulose film such that after washing, the nitrocellulose film may contain biomolecules at one or more of the binding locations and not appreciably in the spaces between the binding locations. The binding locations may also be referred to herein as analyte capture locations.

At 908, method 900 includes processing the microarray. Processing the microarray includes exposing the microarray (with biomarkers from the biological sample bound to the analyte capture regions) to one or more reagents, including a reactive mixture of probe molecules. The probe molecules may be configured to selectively bind to the target biomarkers and/or the biomarker-binding ligand pair. The probe molecules may include a label for detection using either the naked eye or an imaging system. The label may be a fluorescent label, such as an organic fluorophore or a QNC. In some examples, a plurality of fluorescent labels may be used, each of the fluorescent labels included on probe molecules targeting a subset of the biomarkers. The plurality of fluorescent labels may each be spectrally distinct, having a different excitation wavelength and/or emission wavelength, such that a fluorescent signal produced by one fluorescent label is distinguishable from a fluorescent signal produced by the other fluorescent label(s). For example, a first fluorescent label may be used to distinguish IgG antibodies, and a second fluorescent label may be used to distinguish IgM antibodies for a specific disease or condition. As an example, the first fluorescent label may be a far-red fluorophore, such as Alexa Fluor® 647 (which may be excited with 633 nm light and detected within a range of 660-700 nm), and the second fluorescent label may be a near-infrared (NIR) fluorophore, such as Alexa Fluor® 790 (which may be excited with 770 nm light and detected within a range of 800-820 nm). In the present example, the far-red fluorophore (e.g., Alexa Fluor® 647) does not emit light in the 800-820 nm range and is not excited by 770 nm light. Similarly, the NIR fluorophore (e.g., Alexa Fluor® 790) does not emit light within the 660-700 nm range and is not excited by 633 nm light. Alexa Fluor® 647 and Alexa Fluor® 790, as well as other fluorescent dyes in the Alexa Fluor® family, may be purchased from Thermo Fisher Scientific. Notably, using far-red and NIR fluorophores may reduce background fluorescence of the nitrocellulose film and/or biomolecules immobilized on the nitrocellulose film, increasing a signal-to-noise ratio of the microarray during imaging. However, fluorophores of other fluorophore families and spectral ranges may be used, including coumarin-based dyes, rhodamine-based dyes, xanthene-based dyes, cyanine-based dyes, etc. In particular, the selected fluorophores may have an emission wavelength in a range of approximately 500-900 nm in order to minimize the background of nitrocellulose films and/or the biomolecules. Furthermore, organic fluorophores may be selected over QNCs due to lower costs of the organic fluorophores and increased stability. For example, the probe molecules may be stored as highly stable lyophilized pellets, which may be rehydrated just prior to addition to the microarray. However, in other examples, QNCs, fluorescent proteins, or non-fluorescent labels may be used, such as radiolabels or colorimetric labels. In some examples, a combination of organic fluorophores and QNCs may be used, the specific organic fluorophores and QNCs selected to be spectrally distinguishable from one another.

In some examples, processing the microarray may include transferring the substrate between multiple reagent stations, as indicated at 910. For example, each of the reagent stations may be a snap cap tube, a test tube, or well containing a single reagent, which may be a reactive probe mixture, a wash buffer, an imaging buffer, etc. The substrate may be transferred between the reagent stations in a sequence specified by a protocol. Specifically, the nitrocellulose film, including the microarray, may be submerged in the reagent at each reagent station for a predetermined duration and in a predetermined order, with or without shaking and with or without incubation at room temperature or another temperature. Additionally or alternatively, in some examples, processing the microarray may include drawing liquids through a microarray reaction chamber via capillary action, as indicated at 912. For example, the substrate may be included in a cartridge (e.g., cartridge 800 shown in FIGS. 8A-8C) with all of the reagents on-board, where liquids are drawn through to the microarray incubtaion chamber (e.g., reaction chamber 818 of FIGS. 8A and 8D) using capillary action to prime a fluid channel within a fluidic gasket (e.g., fluidic gasket 816 shown in FIGS. 8A and 8D). The reagents may flow through the fluid channel to an absorbent collection pad (e.g., waste collection material 820 shown in FIG. 8), which may wick reagents in sequential order, during which some reactants may be added en route to incubation with the microarray (e.g., lyophilized probe molecules in adjacent chamber 817). Thus, in some examples, processing the microarray may include actuating a blister pack full of liquid reagents (e.g., sealed liquid reagent pack 814 shown in FIG. 8A) and incubating the microarray for a predetermined duration (e.g., 5-30 minutes), with or without shaking.

At 914, method 900 includes imaging the microarray to detect disease biomarkers, as will be described with respect to FIG. 16. Briefly, the microarray may be imaged using an imaging system that is configured to detect the labeled probe molecules attached to the bound biomarkers of interest. As an example, the imaging system may be a fluorescence imager that includes a semi-conductor laser for exciting fluorescent labels and a camera for capturing light emitted by the excited fluorescent labels. The imaging system may further include software for analyzing the captured light and correlating the detected light with the location of biomarkers in the microarray to determine the presence and concentration of specific biomarkers or biomarker combinations indicative of a disease or condition. Examples of such imaging systems, such as biomolecule analysis systems, will be described below with respect to FIGS. 10-15. Following 914, method 900 ends.

Turning now to FIG. 10, a schematic of a first example of a biomolecule analysis system 1000 is shown. The biomolecule analysis system 1000 may include a biomolecule microarray assembly 1010 and a computer 1082. In particular, FIG. 10 shows a two-dimensional schematic diagram showing components of the biomolecule analysis system 1000 and how they may be electrically coupled to one another. As such, the actual sizes and relative positions of the components of the biomolecule microarray assembly 1010 may be different than shown in FIG. 10.

In one example, the biomolecule microarray assembly 1010 may be configured as a protein microarray for quantifying protein levels in one or more of lysate, whole blood, plasma, serum, saliva, CSF, other bodily fluid, environmental or synthetic sample. As such, the biomolecule microarray assembly 1010 may be used in the diagnosis and/or detection of infectious diseases, poisoning, or other contaminations. Specifically, antibody and/or antigen expression levels in a blood sample from a patient may be analyzed using the biomolecule microarray assembly 1010 to identify a disease or condition afflicting the patient. However, it should be appreciated that in other embodiments, the biomolecule microarray assembly 1010 may be configured to image and analyze biomolecules other than proteins, such as DNA, cDNA, mRNA, siRNA, peptides, carbohydrates, lipids, whole cells, etc. Thus, the biomolecule microarray assembly 1010 may be any one of a protein, DNA, RNA, peptide, tissue, antibody, carbohydrate, lipid, or other microarray reader. The biomolecule microarray assembly 1010 may also be referred to herein as biomolecule microarray assay imager 1010.

The biomolecule microarray assembly 1010 may include a microarray chip 1012 or other substrate, a portion or all of which may be coated with a nitrocellulose film 1014 in one or more locations, the nitrocellulose film 1014 including a plurality of analyte capture regions comprising analyte capture locations 1016 which may comprise binding ligands onto which a sample may be loaded. Thus, the binding ligands may be arranged on the analyte capture locations 1016 on the nitrocellulose film 1014 to form a microarray 1015. The nitrocellulose film may be produced according to the method of FIG. 4 and printed with the binding ligands according to the example method of FIG. 9. The microarray 1015 of binding ligands on the analyte capture locations 1016 may chemically bind biomolecules of the sample (e.g., biomarkers) to the chip 1012. In the description herein, the binding ligands may also be referred to as spots 1016. One or more groupings of analyte capture locations with or without additional binding ligands may form analyte capture regions on a nitrocellulose strip. Such analyte capture regions may be contiguous or separated by non-binding regions.

The sample may first be loaded onto the film 1014 prior to insertion of the chip 1012 into the biomolecule microarray assembly 1010 for imaging, such as described above with respect to FIG. 9. The sample may include a plurality of biomolecules, of which only a subset may be desired for analysis. As such, target biomolecules, which comprise the subset of sample biomolecules desired for analysis (e.g., biomarkers), may be tagged with a label such as a fluorescent label. The fluorescent label may be any suitable fluorescent tag that may bind to the target biomolecules and emit light of a different wavelength than a light source in response to excitation from the light source, such as a laser. The microarray 1015 of analyte capture locations 1016 may be referred to as an assay once the biomolecules from the sample have been chemically bound to the analyte capture locations 1016 and have been tagged with the fluorescent label(s).

In some examples, the fluorescent label may include organic dyes such as food dye. In other examples, the fluorescent label may include other fluorescent species such as QNCs, ruthenium-based fluorescent dyes, coumarin-based dyes, rhodamine-based dyes, xanthene-based dyes (e.g., fluorescein), cyanine-based dyes, ethidium bromide, or green fluorescent protein (GFP). It should also be appreciated that multiple types of fluorescent species may be used for the fluorescent label, where each type of fluorescent species may emit a different wavelength of light. Thus, multiple types of labels that emit different wavelengths of light may be utilized in the same assay 1015.

The chip 1012 may be removably coupled to the microarray assembly via a door. The chip 1012 may be relatively planar and may include a flat surface upon which the thin porous nitrocellulose film 1014 is attached. The sample may be deposited onto the microarray 1015 of analyte capture locations 1016 included on the nitrocellulose film 1014. The analyte capture locations 1016 may each be configured to bind to one or more of the biomolecules present in the sample. Thus, the analyte capture locations 1016 may be configured to bind to one or more proteins, in examples where the microarray 1015 is a protein microarray. However, in other examples, the analyte capture locations 1016 may be configured to bind to one or more oligonucleotides in examples where the microarray 1015 is a DNA microarray.

In addition to the porous nitrocellulose, the analyte capture locations 1016 may include one or more binding ligands to which the biomolecules in the sample may bind. Thus, the binding ligands of the analyte capture locations 1016 may affix/immobilize the biomolecules to the analyte capture locations 1016 on the chip 1012. In examples where the biomolecule microarray assembly 1010 is configured as a protein microarray, the analyte capture locations 1016 may comprise purified antigens, to which antibodies in the sample may bind. Thus, antigens may be spotted or otherwise printed onto the nitrocellulose film 1014 at the analyte capture locations 1016. However, it should be appreciated that in other examples, the analyte capture locations 1016 may comprise molecules other than antigens for binding to biomolecules of the sample other than antibodies. For example, the analyte capture locations 1016 may comprise antibodies, antibody fragments, or affibody molecules to which antigens in the sample may bind. In still further examples, the analyte capture locations 1016 may comprise oligonucleotides or cDNA for binding to DNA or RNA in the sample. Alternatively, the analyte capture locations 1016 may comprise only porous nitrocellulose, and may not include antigens or other binding ligands for binding to the sample biomolecules. In such examples, proteins in the sample may bind directly to the porous nitrocellulose at the analyte capture locations 1016.

Additionally or alternatively, a reactive mixture of probe molecules including any of the suitable fluorescent labels may be introduced to the sample biomolecules after the sample biomolecules have adhered to the analyte capture locations 1016 on the chip 1012. Specifically, the probe molecules may include fluorescent labels such as organic fluorophores for fluorescently labeling the biomolecules in the spots 1016. The probe molecules may be configured to selectively bind to the target biomolecules. In this way, only the target biomolecules may be tagged with the fluorescent labels. In examples where the sample includes antibodies and the biomolecule microarray assembly 1010 is configured as a protein microarray, labeled probe molecules may be configured to selectively bind to only target antibodies in the sample. Said another way, the labeled probe molecules may be conjugated to the target antibodies in the sample. Thus, the probe molecules may be introduced to the analyte capture locations 1016 after the target antibodies have affixed to the analyte capture locations 1016 for fluorescently labeling the target antibodies.

To enable the mixing and binding of the biomolecules from the sample with the analyte capture locations 1016, an assay cover 1017 may be positioned over the nitrocellulose film 1014. In some examples, the assay cover 1017 may fully enclose the chip 1012. However, in other examples, the assay cover 1017 may only enclose the nitrocellulose film 1014 and analyte capture locations 1016. One or more of the sample, a blocking solution, washing solution, and/or the reactive mixture may be mixed with the nitrocellulose film 1014 in the assay cover 1017. Further, the assay cover 1017 may fluidically seal the nitrocellulose film 1014 from the environment. Thus, the assay cover 1017 may function as a container, holding various aqueous mixtures which may aid in binding the sample biomolecules to the analyte capture locations 1016, inhibiting binding of and/or removing sample biomolecules that are not the target biomolecules, and fluorescently tagging the target biomolecules. Put more simply, the assay cover 1017 may permit reactions between the nitrocellulose film 1014 and various aqueous mixtures during development of the assay.

Further, the assay cover 1017 may serve to prevent contamination of the sample and film 1014. By enclosing the film 1014 in the assay cover 1017, an amount of foreign materials such as dust, dirt, bacteria, enzymes, fungi, viruses, etc., that accumulate on the film 1014 may be reduced. Specifically, the assay cover 1017 may fluidically seal the film 1014 from the outside environment prior to insertion of the chip 1012 into the biomolecule microarray assembly 1010.

Once the target biomolecules have been fluorescently tagged and chemically bound to the nitrocellulose film 1014, the assay cover 1017 may be removed from the chip 1012, and the film 1014 may be dried. After the biomolecules in the sample have been bound to the nitrocellulose film 1014 and fluorescently labeled, the chip 1012 may be inserted into a cuvette 1013. The cuvette 1013 may fully enclose the chip 1012 prior to insertion of the chip 1012 in the biomolecule microarray assembly 1010. However, in other examples, the cuvette 1013 may be inserted into the biomolecule microarray assembly 1010 prior to insertion of the chip 1012 into the cuvette 1013, and the chip 1012 may therefore be inserted into the cuvette 1013 after the cuvette 1013 has been inserted into the assembly 1010.

Cuvette 1013 may serve a variety of purposes. The cuvette 1013 may fully enclose and/or hold the chip 1012 and center the chip 1012 within the biomolecule array assembly 1010. Thus, the cuvette 1013 may serve as a holder for the chip 1012, which retains the chip 1012 in a fixed position within the biomolecule microarray assembly 1010 during imaging of the analyte capture locations 1016. In some examples, the biomolecule microarray assembly 1010 may include one or more mechanical stabilizers 1041, which may interface with external faces of the cuvette 1013 for retaining the cuvette 1013 in a fixed position within the biomolecule microarray assembly 1010.

In some examples, the assay cover 1017 may not be used to facilitate mixing of the sample and reactive mixture with the chip 1012. In such examples, the cuvette 1013 may provide the same above-mentioned functions as the assay cover 1017. Thus, the cuvette 1013 may in some examples function as a container, holding both the chip 1012 and various aqueous mixtures included to aid in binding the sample biomolecules to the analyte capture locations 1016, inhibit binding of and/or remove sample biomolecules that are not the target biomolecules, and fluorescently tag the target biomolecules. Said another way, the cuvette 1013 may permit reactions between the nitrocellulose film 1014 and various aqueous mixtures during development of the assay.

Further, the cuvette 1013 may serve to prevent contamination of the chip 1012. By fully enclosing the chip 1012 in the cuvette 1013, an amount of foreign materials such as dust, dirt, bacteria, enzymes, fungi, viruses, etc., that accumulate on the chip 1012 may be reduced. Specifically, the cuvette 1013 may fluidically seal the chip 1012 from the outside environment once the chip 1012 is inserted into the cuvette 1013.

The chip 1012 may be inserted into the biomolecule microarray assembly 1010 for imaging of the analyte capture locations 1016 (also referred to as binding locations). Specifically, in some examples where the cuvette 1013 is not included in the assembly 1010, the chip 1012 may be inserted directly into the biomolecule microarray assembly 1010. However, in other examples where the cuvette 1013 is included in the microarray assembly 1010, the cuvette 1013 including the chip 1012 may be inserted into the biomolecule microarray assembly 1010. The cuvette 1013 may include an authentication device which may be either mechanical or electronic, which may engage with the biomolecule microarray assembly 1010 when the cuvette 1013 is inserted. The authentication device may ensure that the biomolecule microarray 1015 is imaged only when the cuvette 1013 is inserted in the biomolecule microarray assembly 1010.

Once the chip 1012 is inserted into the biomolecule microarray assembly 1010, the microarray 1015 comprising the bound and labeled analytes of interest may be imaged by the biomolecule microarray assembly 1010 to visualize positive labeling of the analyte capture locations 1016 (e.g., the analyte capture locations 1016 upon which the target biomarkers and the corresponding probe molecules are bound). Imaging the microarray 1015 may include exciting the fluorescent labels via a laser 1018 and capturing light emitted by the fluorescent labels with a camera 1030. The camera 1030 may be a charge-coupled device (CCD) or a complementary metal-oxide semiconductor (CMOS), for example. The laser 1018 may be a violet diode laser and may emit a light beam in a range of wavelengths between 400 and 450 nanometers. Light emitted from the laser 1018 may travel in a first direction towards a first dichroic mirror 1022, as shown by a light propagation arrow 1019a in FIG. 10. Before reaching the first dichroic mirror 1022, light from the laser 1018 may first pass through a diffusing element 1020 positioned between the first dichroic mirror 1022 and the laser 1018. The diffusing element 1020 may be positioned approximately perpendicular to the direction of light propagation from the laser 1018 to increase a uniformity of the beam profile of the light from the laser 1018. Thus, by including the diffusing element 1020 the uniformity of light intensity across the surface area of the microarray 1015 may be increased. Said another way, the intensity of light received by each analyte capture location 1016 may be relatively the same after passing the laser light through the diffusing element 1020.

Upon reaching the first dichroic mirror 1022, light from the laser 1018 may be reflected by approximately 90 degrees and may propagate toward the chip 1012 in a second direction, as shown by a light propagation arrow 1019b in FIG. 10. Thus, the first dichroic mirror 1022 may be configured to reflect light from the laser while allowing other wavelengths of light to pass through. As such, the first dichroic mirror 1022 may only reflect light of wavelengths between a first range of 400 and 450 nm. Said another way, the first dichroic mirror 1022 may be selectively transparent to light of wavelengths longer than 450 nm. Thus, only light with a wavelength greater than 450 nm may pass through the first dichroic mirror 1022 without being reflected. Further, the first dichroic mirror 1022 may be positioned at approximately a 45 degree angle with respect to the laser 1018, so that light emitted from the laser 1018 is deflected approximately 90 degrees towards the chip 1012. More specifically, a first surface 1021 of the first dichroic mirror 1022, which faces the laser 1018, may be oriented at approximately a 45 degree angle with respect to the first direction of light propagation. Thus, the first dichroic mirror 1022 and a second mirror 1024 may be oriented to that their first surfaces 1021 and 1025, respectively are oriented at a 45 degree angle with respect to a light source end of the laser 1018 from which a laser beam is emitted. Said another way, the laser 1018 may be pointed at the first dichroic mirror 1022 so that light from the laser 1018 may reach the mirror 1022 at an incident angle of approximately 45 degrees. However, in other examples, the mirror 1022 may be angled with respect to the laser 1018 at an angle greater or less than 45 degrees. Further, the first dichroic mirror 1022 and the second mirror 1024 may be oriented so that their first surfaces 1021 and 1025, respectively, are oriented at a 45 degree angle with respect to a lens 1031 of the camera 1030. Additionally or alternatively, the first dichroic mirror 1022 and the second mirror 1024 may be oriented to that their first surfaces 1021 and 1025, respectively, are oriented at a 45 degree angle with respect to a front wall of the cuvette 1013, which may be an optically clear window.

Light from the laser 1018 may reach the chip 1012 and excite the fluorescent labels (e.g., the organic dyes). Said another way, when exposed to light from the laser 1018, the fluorescent labels chemically bound to the target biomolecules may emit light back toward the first dichroic mirror 1022 in a third direction, which may be approximately opposite the second direction, as shown by light propagation arrows 1023a. Light emitted from the laser is shown by the solid arrows 1019a and 1019b, while light emitted from the microarray 1015 is shown by dashed arrows 1023a and 1023b. Emission light from the fluorescent labels of the microarray 1015 may be of a different wavelength than the light from the laser 1018. Specifically, the wavelength of the emission light may be greater than the wavelength of the light emitted from the laser 1018. In some examples, the wavelength of the emission light from the microarray 1015 may be in a range between 500 and 900 nm.

As described above, the first dichroic mirror 1022 may only reflect light emitted from the laser 1018. Thus, since the light emitted from the fluorescent labels of the microarray 1015 may be greater than that from the laser 1018, the light emitted from the fluorescent labels may pass relatively unobstructed through the first dichroic mirror 1022 and onto a second mirror 1024. Thus, as shown in FIG. 10, the microarray 1015, first dichroic mirror 1022, and second mirror 1024 may be relatively aligned with one another along a straight line, with the first dichroic mirror 1022 being positioned between the second mirror 1024 and the microarray 1015. However, light emitted from the labels bound to molecules in the microarray 1015 may be reflected off a first surface 1025 of the second mirror 1024. Thus, the second mirror 1024 and first dichroic mirror 1022 may reflect different wavelengths of light. Specifically, the second mirror 1024 may reflect a second range of wavelengths of light that is greater than the first range of wavelengths reflected by the first dichroic mirror 1022. In some examples, the second mirror 1024 may reflect substantially all wavelengths of light. Thus, the first dichroic mirror 1022 may be configured to reflect electromagnetic waves only with wavelengths up to 450 nm, whereas the second mirror 1024 may be configured to reflect electromagnetic waves up to 8000 nm. Additionally, in some examples, the second mirror 1024 may not reflect light with wavelengths less than 450 nm. In this way, light from the laser 1018 may be reflected by the first dichroic mirror 1022, and light emitted from the labels bound to the biomolecules bound in the microarray 1015 may pass through the first dichroic mirror 1022 without being reflected. However, the second mirror 1024 may be configured to reflect light emitted from the microarray 1015, and as such, upon reaching the first surface 1025 of the second mirror 1024, light emitted from the microarray 1015 may be reflected approximately 90 degrees toward the camera 1030.

Thus, the second mirror 1024 may be oriented at approximately a 45 degree angle with respect to the microarray 1015, in the same or similar orientation as the first dichroic mirror 1022. The second mirror 1024 and the first dichroic mirror 1022 may be oriented substantially parallel to one another. In this way, incident light from the microarray 1015 may be reflected off the first surface 1025 of the second mirror 1024 in a fourth direction, substantially parallel and opposite the first direction as shown by light propagation arrows 1023b. Thus, light reflected off the second mirror 1024 may propagate substantially parallel to and opposite the direction of propagation of light emitted from the laser 1018.

Camera 1030 may therefore be aligned approximately parallel to the laser 1018. Said another way, the camera 1030 and laser 1018 may be positioned on the same side of the first dichroic mirror 1022 and the second mirror 1024. By including the second mirror 1024, and positioning the camera 1030 parallel to the laser 1018, the compactness of the microarray assembly may be increased, and therefore the size of the biomolecule microarray assembly 1010 may be reduced. In this way, the laser 1018 may emit light in a first direction towards the first dichroic mirror 1022, and the camera 1030 may receive light emitted from the microarray 1015 in a fourth direction, the fourth direction opposite the first direction. The camera 1030 may therefore by oriented so that the lens 1031 of the camera 1030 faces the second mirror 1024. Specifically, the lens 1031 may face the first surface 1025 of the second mirror 1024 at approximately a 45 degree angle.

Thus, the camera 1030 may be oriented parallel with respect to the laser 1018 so that the lens 1031 faces the same direction as the source of light from the laser 1018. In some examples, the camera 1030 and laser 1018 may be positioned adjacent to one another such that no additional components separate the laser 1018 and camera 1030. However, in other examples the camera 1030 and laser 1018 may be spaced away from one another. In this way, the parallel arrangement of the laser 1018 and camera 1030 may be referred to herein as an optically folded configuration or arrangement since the light emitted by the laser 1018, and the light received by the camera 1030 propagate in approximately parallel but opposite directions. Said another way, the camera 1030, laser 1018, first dichroic mirror 1022, and second mirror 1024 may be positioned in an optically folded arrangement, so that light emitted from the laser propagates in a parallel and opposite direction to light reflected towards the camera from the second mirror.

Before reaching the camera 1030, light emitted from the microarray 1015 and reflected off the second mirror 1024 may pass through one or more filters. In one example, a bandpass filter 1026 may be positioned between the camera 1030 and the second mirror 1024. Additionally or alternatively, a longpass filter 1028 may be positioned between the camera 1030 and the second mirror 1024. In examples where both the longpass filter 1028 and the bandpass filter 1026 are included in the biomolecule microarray assembly 1010, the bandpass filter 1026 may be positioned more proximate the second mirror 1024 than the longpass filter 1028. Together, the longpass filter 1028 and the bandpass filter 1026 may only be optically clear to a desired range of wavelengths of electromagnetic waves. In this way, only light in the wavelength range of that emitted from the fluorescent labels may pass through the filters 1028 and 1026 en route to the camera 1030. As one example, only light with a wavelength of less than 800 nm may pass through the filters 1028 and 1026. Thus, by including the filters 1026 and 1028, background noise (e.g., light emitted from the microarray 1015 not from the fluorescent labels) may be significantly reduced as compared to microarray assemblies not including the filters.

Additionally, in some examples, the filters may be selectable. Thus, the biomolecule microarray assembly 1010 may be adaptable to multi-color multiplexing and detection of several fluorescent labels by providing multiple filters that may be selectable to filter different wavelengths of light based on the desired fluorescent label. For example, the controller 1134 may turn one light source on and the other off, and change the detection wavelength by actuating a linear slide to change the emission filter, altering the type of fluorescent labels detected. As such, the camera 1030 may be configured to independently detect two or more signals from multiple fluorescent labels of different colors.

Although second mirror 1024 is included in the biomolecule microarray assembly 1010 in the embodiment of the biomolecule microarray assembly 1010 described above, it should also be appreciated that in other embodiments, the biomolecule microarray assembly 1010 may not include second mirror 1024. In such embodiments where second mirror 1024 is not included in the biomolecule microarray assembly 1010, the camera 1030 may be positioned so that the lens 1031 directly faces the microarray 1015. Said another way, the lens 1031 may be positioned perpendicular to the direction of light propagation from the microarray 1015 so that light emitted from the microarray 1015 may pass through the lens 1031 and be captured in an image by the camera 1030. Thus, the camera 1030 may be positioned perpendicular to the laser 1018, so that light emitted from the microarray 1015 may be received and imaged by the camera 1030, after the light passes through the first dichroic mirror 1022. Therefore, in examples where mirror 1024 is not included in the assembly, light emitted by the microarray 1015 may pass in a relatively straight line to the lens 1031 of the camera 1030 without reflection by a mirror. Further, the filters 1026 and 1028 may be positioned between the first dichroic mirror 1022 and the lens 1031 of the camera 1030 in examples where the camera 1030 is positioned perpendicular to the laser 1018 and second mirror 1024 is not included in the biomolecule microarray assembly 1010. Thus, the filters 1026 and 1028, lens 1031, and camera 1030 may be aligned antiparallel to light propagation arrows 1023a for capturing light emitted from the microarray 1015. Said another way, microarray 1015, first dichroic mirror 1022, filters 1026 and 828, lens 1031, and camera 1030 may all be aligned with one another in a substantially straight line perpendicular to the first direction shown by light propagation arrows 1019a.

In this way, the lens 1031 may gather light produced from the fluorescent labels, and the camera 1030 may capture an image of the microarray 1015. The camera 1030 may be a digital camera, configured to acquire digital images of the microarray 1015 and the spots 1016 positioned therein. Specifically, light emitted from the spots 1016 on the microarray 1015 in response to excitation from the laser 1018 may be captured by the camera 1030. The resolution of the image of the microarray 1015 may depend on the distance of the camera 1030 from the microarray 1015, the focal length of the lens 1031, the magnification of the camera 1030, and the surface area of the microarray 1015. In some examples, the camera 1030 may be a 5, 6, 7, 8, 9, 10 or more megapixel camera. However, in another example, the camera 1030 may be a 3 megapixel camera. In still further examples, the megapixel of the camera 1030 may be a range between 2 and 8 megapixels. The focal length of the lens 1031 may be one of 30, 25, 17.5, or 12 mm. However, in other examples the focal length of the lens 1031 may be a range between 5 and 30 mm. A width of the microarray 1015 may in some examples be 25 mm. However, in other examples, the microarray 1015 may have a width of 12.5 mm. In still further examples, the width of the array may be approximately 6.25 mm. Further, the width of the microarray 1015 may be a range between 5 and 30 mm. While the microarray is shown as rectangular, it may be circular, oval, triangular, square or any other desired shape. The optical path length between the microarray 1015 and the camera 1030 may be approximately 120 mm. However, in other examples, the optical path length between the microarray 1015 and the camera 1030 may be greater or less than 120 mm. Thus, the resolution of the images captured by the camera 1030 may be in a range between 2 and 10 microns. Specifically, by reducing the size of the microarray 1015 to a width of approximately 12.5 mm and configuring the camera 1030 with a magnification of 0.45 and lens focal length of 25 mm, the resolution of the image may be increased to 4.9 microns.

Images captured by the camera 1030 may in some examples be transferred to a computer 1082 for analysis. Computer 1082 may be any computing device configured to access a network, including but not limited to a server, personal computer, laptop, smartphone, tablet, and the like. In some examples, the biomolecule microarray assembly 1010 may be electrically coupled to the computer 1082 via a wired connection such as a USB port. In other embodiments, the connection may be wireless. Any wireless transmission protocol may be used including, but not limited to, sub-GHZ, ZigBee, Bluetooth, GSM/LTE, passive RF, or Wi-Fi.

Computer 1082 may include a logic subsystem 1083 and a data-holding subsystem 1084. Computer 1082 may additionally include a display subsystem 1085, a communication subsystem 1086, and/or other components not shown in FIG. 10. For example, computer 1082 may also optionally include peripheral devices including, but not limited to, user input devices such as keyboards, mice, game controllers, cameras, microphones, and/or touch screens.

Logic subsystem 1083 may include one or more physical devices configured to execute one or more instructions. For example, logic subsystem 1083 may be configured to execute one or more instructions that are part of one or more applications, services, programs, routines, libraries, objects, components, data structures, or other logical constructs. Such instructions may be implemented to perform a task, implement a data type, transform the state of one or more devices, or otherwise arrive at a desired result.

Logic subsystem 1083 may include one or more processors that are configured to execute software instructions. Additionally or alternatively, the logic subsystem 1083 may include one or more hardware or firmware logic machines configured to execute hardware or firmware instructions. Processors of the logic subsystem 1083 may be single or multi-core, and the programs executed thereon may be configured for parallel or distributed processing. The logic subsystem 1083 may optionally include individual components that are distributed throughout two or more devices, which may be remotely located and/or configured for coordinated processing. One or more aspects of the logic subsystem 1083 may be virtualized and executed by remotely accessible networking computing devices configured in a cloud computing configuration.

Data-holding subsystem 1084 may include one or more physical, non-transitory devices configured to hold data and/or instructions executable by the logic subsystem 1083 to implement the herein described methods and processes. When such methods and processes are implemented, the state of data-holding subsystem 1084 may be transformed (for example, to hold different data). For example, the data holding subsystem 1084 may be configured to store images from the camera 1030. The images may be modified and/or analyzed based on instructions stored in the logic subsystem 1083.

Data-holding subsystem 1084 may include removable media and/or built-in devices. Data-holding subsystem 1084 may include optical memory (for example, CD, DVD, HD-DVD, Blu-Ray Disc, etc.) and/or magnetic memory devices (for example, hard drive disk, floppy disk drive, tape drive, MRAM, etc.) and/or non-volatile memory cards (for example, SD and micro-SD cards), flash memory such as a USB drive and the like. Data-holding subsystem 1084 may include devices with one or more of the following characteristics: volatile, nonvolatile, dynamic, static, read/write, read-only, random access, sequential access, location addressable, file addressable, and content addressable. In some embodiments, logic subsystem 1083 and data-holding subsystem 1084 may be integrated into one or more common devices, such as an application-specific integrated circuit or a system on a chip. Together, the logic subsystem 1083 and data-holding subsystem 1084 may be configured to store images captured from the camera 1030 and analyze the images to determine a presence, absence, or amount of biomarkers in each of the analyte capture location 1016 of the microarray 1015.

Thus, the logic subsystem 1083 may include one or more algorithms for processing and analyzing data received from the camera 1030. Thus, in order to compare the intensity of different wavelengths of light emitted from the labeled analytes in the analyte capture locations 1016, the logic subsystem 1083 may include one or more algorithms or software for image analysis. One or more of a combination of different algorithms for data normalization and statistical techniques, such as artificial neural networks, multivariate statistics, machine learning such as, e.g., pixel feature classification, and tree algorithms may be stored in the logic subsystem 1083 for analyzing the images captured by the camera 1030 to detect for and/or quantify biomarkers in the sample based on relative intensities of different wavelengths of light. In this way, in examples where the biomolecule microarray assembly 1010 is configured as a protein microarray, light received from the analyte capture locations 1016 and captured by the camera 1030 may be compared and analyzed to detect biomarkers of infectious diseases. However, it should be appreciated that the images may also be analyzed to detect and/or quantify gene expression levels, oligonucleotides, antibodies, antigens, etc.

When included, display subsystem 1085 may be used to present a visual representation of data held by data-holding subsystem 1084. As the herein described methods and processes change the data held by the data-holding subsystem 1084, and thus transform the state of the data-holding subsystem 1084, the state of display subsystem 1085 may likewise be transformed to visually represent changes in the underlying data. For example, the images captured by the camera 1030 may be displayed to a user via the display subsystem 1085. Further, modifications to and/or analysis of the images may be displayed to the user via the display subsystem 1085. Display subsystem 1085 may include one or more display devices utilizing virtually any type of technology. Such display devices may be combined with logic subsystem 1083 and/or data-holding subsystem 1084 in a shared enclosure, or such display devices may be peripheral display devices.

When included, communication subsystem 1086 may be configured to communicatively couple computer 1082 with one or more other computing devices, such as a controller 1034 of the biomolecule microarray assembly 1010. Thus, in some examples, the microarray assembly 1010 may be electrically coupled to the computer 1082 by a wired connection as shown by the dotted line in FIG. 10. However, in other examples, the biomolecule microarray assembly 1010 may be wirelessly coupled to the computer 1082 via any suitable wireless connection for data transfer such as sub-GHZ, ZigBee, Bluetooth, GSM/LTE, passive RF, or Wi-Fi. In some examples, biomolecule microarray assembly 1010 may transfer information directly to a remote location such as in the cloud. Additionally or alternatively, in some examples, the biomolecule microarray assembly 1010 may include a memory chip 1033, which may be removably coupled to biomolecule microarray assembly 1010 for storing images captured by the camera 1030. The memory chip 1033 may be any suitable memory storage device, such as a USB memory stick, SD card, micro-SD card, etc. Images and/or analyses may be transferred from the memory chip 1033 onto the computer 1082 by removing the memory chip 1033 from the microarray assembly and inserting the memory chip 1033 into the computer 1082 or other suitable device.

Communication subsystem 1086 may include wired and/or wireless communication devices compatible with one or more different communication protocols. As non-limiting examples, communication subsystem 1086 may be configured for communication via a wireless telephone network, a wireless local area network, a wired local area network, a wireless wide area network, a wired wide area network, etc. In some embodiments, communication subsystem 1086 may allow computer 1082 to send and/or receive messages to and/or from other devices, such as controller 1034, via a network such as the public Internet.

Although images from the camera 1030 may be transferred to the computer 1082 for analysis, it should be appreciated that in other embodiments, the biomolecule microarray assembly 1010 may perform image analysis on its own. For example, the controller 1034 of the biomolecule microarray assembly 1010 may include computer readable instructions for analysis of the images captured by the camera 1030. Thus, the controller 1034 may be configured and may function the same as or similarly to the logic subsystem 1083 and data-holding subsystem 1084 of the computer 1082 described above. Said another way, logic subsystem 1083 and data-holding subsystem 1084 may be included in the controller 1034 of the biomolecule microarray assembly 1010 for processing and analyzing the images captured by the camera 1030. In such examples, the microarray assembly 1010 may not be electrically coupled to the computer 1082. Further, in examples where image analysis is performed by the biomolecule microarray assembly 1010, the microarray assembly may further include a display, such as display subsystem 1085.

Controller 1034 may be configured to operate one or more of the laser 1018 and the camera 1030 for capturing images of the microarray 1015. In some examples, the controller 1034 may be configured to receive input from a user via either the computer 1082 or a display screen or button pad located on the biomolecule microarray assembly 1010. Thus, a user may send signals to the controller 1034 via either the computer 1082 or through a display screen or button pad located on the biomolecule microarray assembly 1010. Based on signals received from the user, the controller 1034 may send signals to the laser 1018 to emit light and/or to the camera 1030 for capturing an image of the microarray 1015, as further described with respect to FIG. 16.

Power to the controller 1034, laser 1018, and camera 1030 may be provided by the computer 1082. However, in other examples, the biomolecule microarray assembly 1010 may include a battery 1032 for powering the various components of the biomolecule microarray assembly 1010. The battery 1032 may be any suitable battery such as lithium-ion, lead-acid, solid polymer electrolyte, molten salt, etc. The battery 1032 may be a rechargeable battery. The battery 1032 may provide electrical power to the laser 1018, where the current of said electrical power may be any current level within a range of currents between 1-2 amps. In still further examples, the microarray assembly 1010 may include a power cord for receiving power from an electrical socket.

Although biomolecule microarray assembly 1010 is described as being capable of imaging and/or analyzing fluorescent microarray assays in the example of FIG. 10, it should also be appreciated that other types of assays may be imaged and/or analyzed using the microarray assembly 1010. For example, the biomolecule microarray assembly 1010 may be configured to image and/or analyze colorimetric assays. In such examples, the biomolecule microarray assembly 1010 may include a white light source in place of laser 1018. Thus, a source of white light may be used to illuminate the microarray 1015 instead of the laser 1018. Further, the first dichroic mirror 1022 may be replaced with a beam splitter capable of reflecting only a portion of the white light incident on it from the white light source to the microarray 1015. The orientation and position of the white light source within the biomolecule microarray assembly 1010 may be the same or similar to the laser 1018. Similarly, the orientation and position of the beam splitter may be the same or similar to first dichroic mirror 1022. In this way, white light from the white light source may be reflected onto the microarray 1015 by the beam splitter.

The chromophores or fluorophores bound to the biomarkers in the assay may absorb certain wavelengths of light while reflecting others. The colors reflected by the chromophores or fluorophores may determine the wavelengths of light captured in the image of the array by the camera 1030. The colors captured by the camera may also be adjusted by various filters, such as filters 1026 and 1028. For example, one or more filters may be utilized to exclude certain wavelengths of light reflected from the array and may only allow a desired range of wavelengths to pass through to the camera 1030 for image acquisition. En route to the filters, at least a portion of the light reflected from the microarray 1015 may pass through the beam splitter and on to the second mirror 1024, since the beam splitter may only reflect a portion of incident light, and may allow the remaining portion of incident light not reflected to pass therethrough. In this way, by allowing light to be both reflected and transmitted, the beam splitter may serve to reflect at least a portion of the white light emitted by the white light source to the microarray 1015 for illuminating the chromophores or fluorophores in the assay. Additionally, the beam splitter may serve to allow at least a portion of the light reflected by the microarray 1015 to pass through the beam splitter and on to the filters and camera 1030 for imaging of the microarray 1015.

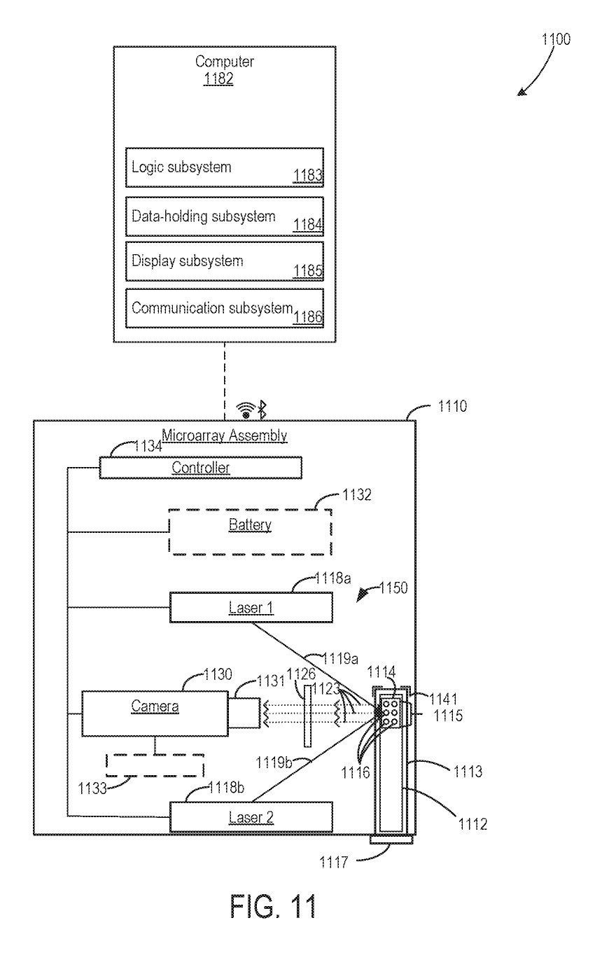

Next, FIG. 11 shows a schematic of a second embodiment of an example biomolecule analysis system 1100. The biomolecule analysis system 1100 may include a biomolecule microarray assembly 1110 and a computer 1182. In particular, FIG. 11 shows a two-dimensional schematic diagram showing components of the biomolecule analysis system 1100 and how they may be electrically coupled to one another. As such, the actual sizes and relative positions of the components of the biomolecule microarray assembly 1110 may be different than shown in FIG. 11. Similarly to the first embodiment of an example biomolecule analysis system 1100 shown in FIG. 10, biomolecular microarray assembly 1110 may be configured as an imaging system for detecting disease biomarkers in a biological sample from a patient.

The microarray assembly 1110 may include a flat substrate 1112 which has been shown in further detail in FIG. 5. A portion or all of the flat substrate may be coated with a nitrocellulose film 1114, the nitrocellulose film 1114 including a plurality of analyte capture regions comprising analyte capture locations or wells 1116 in which binding ligands may be placed and onto which the biological sample may be loaded. In the description herein, the binding ligands located in the analyte capture locations or wells 1116 may also be referred to as spots 1116.

In addition to the porous nitrocellulose, the analyte capture locations 1116 may include one or more binding ligands to which the biomarkers in the sample may bind. The nitrocellulose film may be produced according to the method of FIG. 4 and binding ligands may be printed in the analyte capture locations 1116 according to the example method of FIG. 9. Thus, the binding ligands of the analyte capture locations 1116 may affix/immobilize the biomarkers in the sample to the analyte capture locations 1116 on the flat substrate 1112. In examples where the microarray assembly 1110 is configured as a protein microarray, the analyte capture locations 1116 may comprise purified antigens, to which antibodies in the sample may bind. Thus, antigens may be spotted onto the nitrocellulose film 1114 at the analyte capture locations 1116. However, it should be appreciated that in other examples, the analyte capture locations 1116 may comprise molecules other than antigens for binding to biomolecules of the sample other than antibodies. For examples, the analyte capture locations 1116 may comprise antibodies, antibody fragments, or affibody molecules to which antigens in the sample may bind. In still further examples, the analyte capture locations 1116 may comprise oligonucleotides or cDNA for binding to DNA or RNA in the sample. Alternatively, the analyte capture locations 1116 may comprise only porous nitrocellulose, and may not include antigens or other binding ligands for binding to the sample biomolecules. In such examples, proteins in the sample may bind directly to the porous nitrocellulose at the analyte capture locations 1116.

The sample may first be loaded onto the film 1114 prior to insertion of the flat susbstrate 1112 into the microarray assembly 1110 for imaging, such as described above with respect to FIG. 9. As also described above, the sample may include one or more bodily fluids that include antigens and/or antibodies of interest for infectious and/or non-infectious disease diagnosis. The sample may also be an environmental sample. Thus, the sample may include a plurality of biomolecules, of which only a subset may be desired for analysis. As such, target biomarkers, which comprise the subset of sample biomolecules desired for analysis, may be tagged with a label such as a fluorescent label. The fluorescent label may be any suitable fluorescent tag that may bind to the biomarkers and emit light of a different wavelength than a light source in response to excitation from the light source, such as a laser. The microarray 1115 of analyte capture locations 1116 may be referred to as an assay once the biomarkers from the sample have been chemically bound to the analyte capture locations 1116 and have been tagged with the label(s).

In some examples, after the biomarkers in the sample have been bound to the nitrocellulose film 1114 and fluorescently labeled, the flat substrate 1112 may be inserted into a sheath 1113 which may cover all or a portion of the flat substrate 1112, such as the portion including the nitrocellulose film 1114. The sheath 1113 may enclose the flat substrate 1112 prior to insertion of the flat substrate 1112 in the microarray assembly 1110 via a door 1117. However, in other examples, the sheath 1113 may be inserted into the assembly 1110 prior to insertion of the flat substrate 1112 into the sheath 1113, and the flat substrate 1112 may therefore be inserted into the sheath 1113 after the sheath 1113 has been inserted into the assembly 1110 via a door 1117.

Sheath 1113 may serve a variety of purposes. In some examples, the sheath 1113 may fully enclose and/or hold the flat substrate 1112 and center the flat substrate 1112 within the assembly 1110. Thus, the sheath 1113 may serve as a holder for the flat substrate 1112, which retains the flat substrate 1112 in a fixed position within the assembly 1110 during imaging of the analyte capture locations 1116. In some examples, the assembly 1110 may include one or more mechanical stabilizers 1141, which may interface with external faces of the sheath 1113 for retaining the sheath 1113 in a fixed position within the assembly 1110. Furthermore, in some examples, the sheath 1113 may function as a container, holding both the flat substrate 1112 and various aqueous mixtures included to aid in binding the sample biomolecules to the analyte capture locations 1116, inhibit binding of and/or remove sample biomolecules that are not the target biomolecules, fluorescently tag the biomarkers, and/or place the fluorescently labeled microarray 1115 within an imaging buffer. For example, some fluorescent species are only fluorescent in a specific pH range, and so the imaging buffer may ensure that the fluorescent species are maintained in an environment that enables fluorescence detection.

Further, the sheath 1113 may serve to prevent contamination of the flat substrate 1112 and the microarray 1115. By fully enclosing microarray 1115 in the sheath 1113, an amount of foreign materials such as dust, dirt, bacteria, enzymes, fungi, viruses, etc., that accumulate on the microarray 1115 may be reduced. Specifically, the sheath 1113 may fluidically seal the microarray 1115 from the outside environment once the flat substrate 1112 is inserted into the sheath 1113.

The flat substrate 1112 may be inserted into the microarray assembly 1110 for imaging of the analyte capture locations 1116. Specifically, in some examples where the sheath 1113 is not included in the assembly 1110, the flat substrate 1112 may be inserted directly into the microarray assembly 1110. However, in other examples where the sheath 1113 is included in the microarray assembly 1110, the sheath 1113 including the flat substrate 1112 may be inserted into the microarray assembly 1110. The sheath 1113 may include an authentication device which may be either mechanical or electronic, which may engage with the microarray assembly 1110 when the sheath 1113 is inserted. The authentication device may ensure that the microarray 1115 is imaged only when the flat substrate 1112 with the sheath 1113 is inserted in the microarray assembly 1110. Furthermore, by closing the door 1117 once the flat substrate 1112 is inserted, the flat substrate 1112 may be secured in place, for example using opening 518, further preventing dust and contamination (e.g., onto the sheath 1113) and preventing flat substrate movement or breakage.

Once the flat substrate 1112 is inserted into the microarray assembly 1110, the microarray 1115 may be imaged by the microarray assembly 1110 to visualize positive labeling of the analyte capture locations 1116 (e.g., the analyte capture locations 1116 that are bound to target biomarkers and the corresponding probe molecules). Imaging the microarray 1115 may include exciting the fluorescent labels via a laser and capturing light emitted by the fluorescent labels with a camera 1130. The camera 1130 may be a charge-coupled device (CCD) or a complementary metal-oxide semiconductor (CMOS), for example. The microarray assembly 1110 is shown having a first laser 1118a and a second laser 1118b, which may each emit light of a different wavelength. In some examples, each excitation source, such as the laser, may be matched to a specific fluorescent dye, where each dye has different distinguishable emission wavelengths and excitation wavelengths. As an example, the first laser 1118a may be a 633 nm laser line, and the second laser 1118b may be a 770 nm laser line. In other examples, more than two lasers may be included. In still other examples, one laser may be included. One of the first laser 1118a and the second laser 1118b may be activated during imaging via a controller 1134.

Light emitted from the first laser 1118a may travel in a first direction toward microarray 1115, as shown by a light propagation arrow 1119a in FIG. 11. Before reaching the microarray 1115, light from the first laser 1118a may optionally first pass through one or more optical elements, including diffusing elements, focusing elements, anti-reflective elements and/or mirrors. For example, a diffusing element may increase a uniformity of light intensity across the surface area of the microarray 1115. However, in other examples, light emitted from the first laser 1118a may travel directly to the microarray 1115 without passing through additional optical elements. For example, as shown in FIG. 11, light emitted from the first and second lasers 1118a and 1118b does not interact with a mirror or other optical elements, but travels directly to the microarray 1115 enclosed in a sheath 1113. As such, an optical path length between the first laser 1118a and the microarray 1115 may be reduced, and thus a size of the microarray assembly 1110 may be reduced. Similarly, light emitted from the second laser 1118b may travel in a second direction toward microarray 1115, as shown by a light propagation arrow 1119b in FIG. 11. Before reaching the microarray 1115, light from the second laser 1118b may optionally first pass through one or more optical elements, including diffusing elements, focusing elements, and/or mirrors. However, in other examples, light emitted from the second laser 1118b may travel directly to the microarray 1115 without passing through additional optical elements. As such, an optical path length between the second laser 1118b and the microarray 1115 may be reduced, and thus a size of the microarray assembly 1110 may be reduced. The lasers may be operated simultaneously or independently.

Light from either the first laser 1118a or the second laser 1118b may reach the flat substrate 1112 and excite the fluorescent labels. When exposed to light from either the first laser 1118a or the second laser 1118b, the fluorescent labels chemically bound to the target biomarkers may emit light toward the camera 1130 in a third direction, which may be angled with respect to the first and second directions, as shown by light propagation arrows 1123. Light emitted from the lasers is shown by the solid arrows 1119a and 1119b, while light emitted from the microarray 1115 is shown by dashed arrows 1123. Emission light from the fluorescent labels of the microarray 1115 may be of a different wavelength than the light from each of the first and second lasers 1118a and 1118b. Specifically, the wavelength of the emission light may be greater than the wavelength of the light emitted from the laser line used to excite the fluorescent labels. In some examples, the wavelength of the emission light from the microarray 1115 may be in a range between 500 and 900 nm, depending on the excitation wavelength used. As shown in FIG. 11, the microarray 1115 and the camera 1130, including a lens 1131, may be relatively aligned with one another along a straight line. Camera 1130 may therefore be aligned at an angle with respect to light propagation arrows 1119a and 1119b of the first laser 1118a and the second laser 1118b, respectively.

In some examples, the camera 1130 may be positioned adjacent to each of the lasers such that no additional components separate the lasers 1118a and 1118b and camera 1130. However, in other examples the camera 1130 and the lasers 1118a and 1118b may be spaced away from one another. By positioning the camera 1130 aligned with the microarray 1115, the size of the microarray assembly 1110 may be reduced. As a non-limiting example, the microarray assembly 1110 may be 240 mm in length, 175 mm in width, and 50 mm in height. In other examples, lasers 1118a and 1118b may be arranged at an angle to the sample captured on the nitrocellulose film 1114 and the camera lens may be placed facing the nitrocellulose film 1114 with filters arranged between the camera lens and the sample, positioned to filter light emitted by the sample on the nitrocellulose film 1114 after excitation.

Therefore, before reaching the camera 1130, light emitted from the microarray 1115 may pass through the one or more filters. In the example of FIG. 11, a switchable detection filter in a lens mount 1126 is shown positioned between microarray 1115 and lens 1131. For example, lens mount 1126 may include one or more bandpass filters, longpass filters, and/or shortpass filters. As one example, the lens mount 1126 may include two or more bandpass filters, each of the bandpass filters configured to allow light of a specific wavelength range to pass through to lens 1131. As an example, a first bandpass filter may enable light within a range of 660-700 nm to pass through to lens 1131 while light with wavelengths shorter than 660 nm and longer than 700 nm may be filtered out, and a second bandpass filter may enable light within a range of 800-820 nm to pass through to lens 1131 while light with wavelengths shorter than 800 nm and longer than 820 nm are filtered out. As another example, the switchable detection filter may include one bandpass filter and one longpass filter. For example, the bandpass filter may enable light within a range of 660-700 nm to pass through to lens 1131, while the longpass filter may allow wavelengths of light above 800 nm to pass through to lens 1131. In this way, the assembly 1110 may be adaptable to multi-color multiplexing and detection of several different fluorescent labels by selecting a position of the switchable detection filter 1126a in the lens mount 1126 (e.g., via the controller 1134) to filter different wavelengths of light based on the desired fluorescent label. As such, the camera 1130 may be configured to independently detect two or more signals from multiple fluorescent labels of different colors.

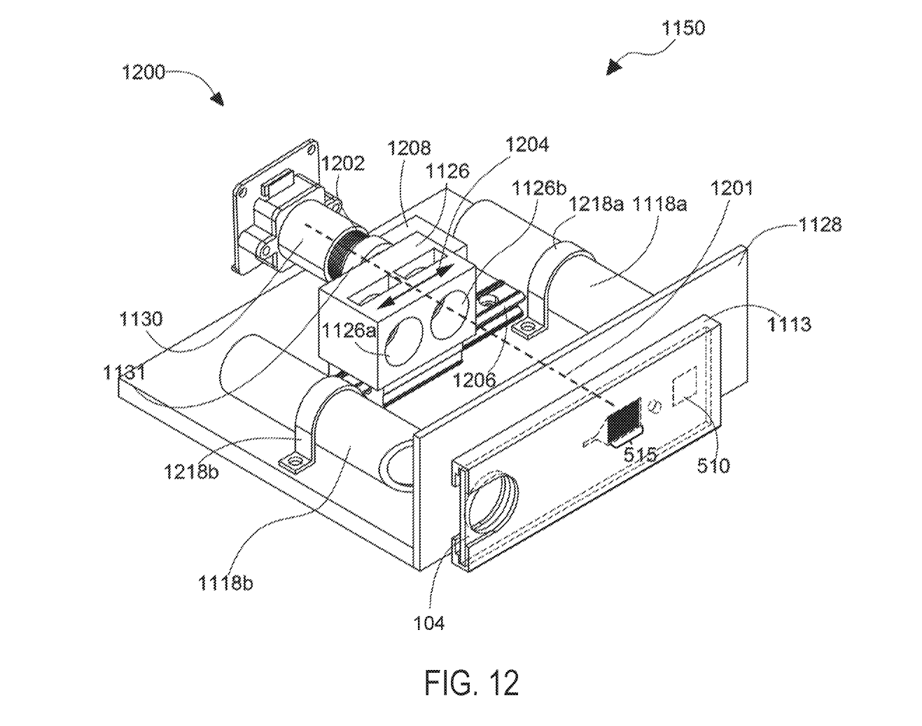

Together, camera 1130 (including lens 1131), lasers 1118a and 1118b, and lens mount 1126 may be included in an optical assembly 1150. A compactness of the optical assembly 1150 helps reduce the size of the microarray assembly 1110. As a non-limiting example, the optical assembly 1150 may have a 100 mm by 100 mm footprint and a 40 mm height. The optical assembly 1150 will be further illustrated in FIG. 12.

In this way, the lens 1131 may gather light produced from the fluorescent labels, and the camera 1130 may capture an image of the emissions from the fluorescent tags bound to target molecules bound in the spots in the microarray. The camera 1130 may be a digital camera, configured to acquire digital images of the light emitted by the spots 1116 positioned in the microarray 1115. Specifically, light emitted from the spots 1116 in response to excitation from the first laser 1118a or the second laser 1118b may be captured by the camera 1130. The resolution of the image of the microarray 1115 may depend on the distance of the camera 1130 from the microarray 1115, the focal length of the lens 1131, the magnification of the camera 1130, and the surface area of the microarray 1115. In some examples, the camera 1130 may be an 8 megapixel camera. However, in another example, the camera 1130 may be a 3 megapixel camera. In still further examples, the megapixel of the camera 1130 may be a range between 2 and 10 megapixels. The focal length of the lens 1131 may be one of 30, 25, 17.5, or 12 mm. However, in other examples the focal length of the lens 1131 may be a range between 5 and 30 mm. A width of the microarray 1115 may in some examples be 25 mm. However, in other examples, the microarray 1115 may have a width of 12.5 mm. In still further examples, the width of the array may be approximately 6.25 mm. Further, the width of the microarray 1115 may be a range between 5 and 30 mm. The optical path length between the microarray 1115 and the camera 1130 may be approximately 120 mm. However, in other examples, the optical path length between the microarray 1115 and the camera 1130 may be greater or less than 120 mm. Thus, the resolution of the images captured by the camera 1130 may be in a range between 2 and 10 microns. Specifically, by reducing the size of the microarray 1115 to a width of approximately 12.5 mm and configuring the camera 1130 with a magnification of 0.45 and lens focal length of 25 mm, the resolution of the image may be increased to 4.9 microns.

Images captured by the camera 1130 may in some examples be transferred to the computer 1182 for analysis. Computer 1182 may be any computing device, including but not limited to a server, personal computer, laptop, smartphone, tablet, and the like. In some examples, the microarray assembly 1110 may be electrically coupled to the computer 1182 via a wired connection such as a USB port through access ports 1314, as further described below. In other examples, the microarray assembly 1110 may be coupled to the computer 1182 via a wireless transmission protocol. Any wireless transmission protocol may be used including, but not limited to, sub-GHZ, ZigBee, Bluetooth, GSM/LTE, passive RF, or Wi-Fi. In some examples, the data may be sent directly to the cloud via a network.

Computer 1182 may include a logic subsystem 1183 and a data-holding subsystem 1184. Computer 1182 may additionally include a display subsystem 1185, a communication subsystem 1186, and/or other components not shown in FIG. 11. For example, computer 1182 may also optionally include user input devices such as keyboards, mice, game controllers, cameras, microphones, and/or touch screens.

Logic subsystem 1183 may include one or more physical devices configured to execute one or more instructions. For example, logic subsystem 1183 may be configured to execute one or more instructions that are part of one or more applications, services, programs, routines, libraries, objects, components, data structures, or other logical constructs. Such instructions may be implemented to perform a task, implement a data type, transform the state of one or more devices, or otherwise arrive at a desired result.

Logic subsystem 1183 may include one or more processors that are configured to execute software instructions. Additionally or alternatively, the logic subsystem 1183 may include one or more hardware or firmware logic machines configured to execute hardware or firmware instructions. Processors of the logic subsystem 1183 may be single or multi-core, and the programs executed thereon may be configured for parallel or distributed processing. The logic subsystem 1183 may optionally include individual components that are distributed throughout two or more devices, which may be remotely located and/or configured for coordinated processing. One or more aspects of the logic subsystem 1183 may be virtualized and executed by remotely accessible networking computing devices configured in a cloud computing configuration.

Data-holding subsystem 1184 may include one or more physical, non-transitory devices configured to hold data and/or instructions executable by the logic subsystem 1183 to implement the herein described methods and processes. When such methods and processes are implemented, the state of data-holding subsystem 1184 may be transformed (for example, to hold different data). For example, the data holding subsystem 1184 may be configured to store images from the camera 1130. The images may be modified and/or analyzed based on instructions stored in the logic subsystem 1183.

Data-holding subsystem 1184 may include removable media and/or built-in devices. Data-holding subsystem 1184 may include optical memory (for example, CD, DVD, HD-DVD, Blu-Ray Disc, etc.) and/or magnetic memory devices (for example, hard drive disk, floppy disk drive, tape drive, MRAM, etc.), flash memory, or non-volatile memory cards and the like. Data-holding subsystem 1184 may include devices with one or more of the following characteristics: volatile, nonvolatile, dynamic, static, read/write, read-only, random access, sequential access, location addressable, file addressable, and content addressable. In some embodiments, logic subsystem 1183 and data-holding subsystem 1184 may be integrated into one or more common devices, such as an application-specific integrated circuit or a system on a chip. Together, the logic subsystem 1183 and data-holding subsystem 1184 may be configured to store images captured from the camera 1130 and analyze the images to determine a presence, absence, or amount of biomarkers in each of the analyte capture locations 1116 of the microarray 1115.

Thus, the logic subsystem 1183 may include one or more algorithms for processing and analyzing data received from the camera 1130. In order to compare the intensity of different wavelengths of light emitted from the different analyte capture locations 1116, the logic subsystem 1183 may include one or more algorithms or software for image analysis. One or more of a combination of different algorithms for data normalization and statistical techniques, such as artificial neural networks, multivariate statistics, machine learning, and tree algorithms may be stored in the logic subsystem 1183 for analyzing the images captured by the camera 1130 to detect for and/or quantify biomarkers in the sample based on relative intensities of different wavelengths of light. In this way, in examples where the microarray assembly 1110 is configured as a protein microarray, light received from the analyte capture locations 1116 and captured by the camera 1130 may be compared and analyzed to look for biomarkers of infectious diseases. However, it should be appreciated that the images may be analyzed to detect and/or quantify gene expression levels, oligonucleotides, antibodies, antigens, etc.

When included, display subsystem 1185 may be used to present a visual representation of data held by data-holding subsystem 1184. As the herein described methods and processes change the data held by the data-holding subsystem 1184, and thus transform the state of the data-holding subsystem 1184, the state of display subsystem 1185 may likewise be transformed to visually represent changes in the underlying data. For example, the images captured by the camera 1130 may be displayed to a user via the display subsystem 1185. Further, modifications to and/or analysis of the images may be displayed to the user via the display subsystem 1185. Display subsystem 1185 may include one or more display devices utilizing virtually any type of technology. Such display devices may be combined with logic subsystem 1183 and/or data-holding subsystem 1184 in a shared enclosure, or such display devices may be peripheral display devices.

When included, communication subsystem 1186 may be configured to communicatively couple computer 1182 with one or more other computing devices, such as controller 1134 of the microarray assembly 1110. Thus, images taken from the camera 1130 may be transferred to the computer 1182 via either a wired electrical connection and/or a wireless connection. Additionally or alternatively, in some examples, the microarray assembly 1110 may include a memory chip 1133, which may be removably coupled to assembly 1110 for storing images captured by the camera 1110. The memory chip 1133 may be any suitable memory storage device, such as a USB memory stick, SD card, micro-SD card, etc. Images may be transferred from the memory chip 1133 onto the computer 1182 by removing the memory chip 1133 from the microarray assembly and inserting the memory chip 1133 into the computer 1182.

Communication subsystem 1186 may include wired and/or wireless communication devices compatible with one or more different communication protocols. Although images from the camera 1130 may be transferred to the computer 1182 for analysis, it should be appreciated that in other embodiments, the microarray assembly 1110 may perform image analysis on its own. For example, the controller 1134 of the microarray assembly 1110 may include computer readable instructions for analysis of the images captured by the camera 1130. Thus, the controller 1134 may be configured and may function the same as or similar to the logic subsystem 1183 and data-holding subsystem 1184 of the computer 1182 described above. Said another way, logic subsystem 1183 and data-holding subsystem 1184 may be included in the controller 1134 of the microarray assembly 1110 for processing and analyzing the images captured by the camera 1130. In such examples, the microarray assembly 1110 may not be electrically coupled to the computer 1182. Further, in examples where image analysis is performed by the microarray assembly 1110, the microarray assembly may further include a display, such as display subsystem 1185.

Controller 1134 may be configured to operate one or more of the first laser 1118a, the second laser 1118b, the camera 1130, and the lens mount 1126 containing detection filters for capturing images of the microarray 1115. In some examples, the controller 1134 may be configured to receive input from a user via either the computer 1182 or a display screen or button pad located on the microarray assembly 1110. Thus, a user may send signals to the controller 1134 via either the computer 1182 or through a display screen or button pad located on the microarray assembly 1110. Based on signals received from the user, the controller 1134 may send signals to one of the first laser 1118a and the second laser 1118b for emitting light, the lens mount 1126 to position the filter, and/or to the camera 1130 for capturing an image of the microarray 1115, as further described with respect to FIG. 16.

Power to the controller 1134, lasers 1118a and 1118b, and camera 1130 may be provided by the computer 1182. However, in other examples, the microarray assembly 1110 may include a battery 1132 for powering the various components of the microarray assembly 1110. The battery 1132 may be any suitable battery such as lithium-ion, lead-acid, solid polymer electrolyte, molten salt, etc. The battery 1132 may be a rechargeable battery. The battery 1132 may provide electrical power to the lasers 1118a and 1118b, where the current of said electrical power may be any current level within a range of currents between 1-2 amps. In still further examples, the microarray assembly 1110 may include a power cord for receiving power from an electrical socket and/or one or more external 12 V batteries. As such, microarray assembly 1110 may be powered via the internal battery 1132 and/or external batteries for increased portability.

Although microarray assembly 1110 is described as being capable of imaging and/or analyzing fluorescent microarray assays in the example of FIG. 11, it should also be appreciated that other types of assays may be imaged and/or analyzed using the microarray assembly 1110. For example, the microarray assembly 1110 may be configured to image and/or analyze colorimetric assays. In such examples, the microarray assembly 1110 may include a white light source in place of lasers 1118a and 1118b. Thus, a source of white light may be used to illuminate labeled ligands bound to the microarray 1115 instead of the lasers 1118a and 1118b. The orientation and position of the white light source within the assembly 1110 may be the same or similar to one of the first laser 1118a and the second laser 1118b.

The chromophores or fluorophores bound to the biomarkers in the assay may absorb certain wavelengths of the white light while reflecting others. The colors reflected by the chromophores or fluorophores may determine the wavelengths of light captured in the image of the microarray 1115 by the camera 1130. The colors captured by the camera may also be adjusted by various filters, such as those in lens mount 1126. For example, one or more filters may be utilized to exclude certain wavelengths of light reflected from the array and may only allow a desired range of wavelengths to pass through to the camera 1130 for image acquisition.

Next, FIG. 12 shows an angled top view 1200 of the optical assembly 1150 introduced in FIG. 11, which may be included in the microarray assembly 1110. Components of FIG. 12 that function the same as components in FIG. 5 and/or FIG. 11 are numbered the same and may not be reintroduced. Similar to FIG. 11, camera 1130 is shown aligned with microarray 1115 on a common axis 1201, with one or more filters, shown here as filter 1126a and 1126b in lens mount 1126, positioned between lens 1131 and microarray 1115. First laser 1118a and second laser 1118b are shown positioned off of the common axis 1201, such as at an angle with respect to microarray 1115. While lasers 1118a and 1118b are shown with only an anti-reflective element between the path of the emitted light and the microarray 1115 enclosed in a sheath 1113, light from the first laser 1118a and second laser 1118a may optionally first pass through one or more additional optical elements, including diffusing elements, focusing elements, anti-reflective elements and/or mirrors. In other aspects there are no additional elements between the path of the emitted light from the lasers and the microarray 1115.

Furthermore, first laser 1118a, second laser 1118b, camera 1130, and lens mount 1126 may be coupled to a platform 1202 so that relative positions of the components are maintained, thereby maintaining optical alignments for exciting fluorescent labels on the microarray 1115 with the lasers 1118a and 1118b and capturing the light emitted by the fluorescent labels with the camera 1130. For example, first laser 1118a and second laser 1118b may be held in place with mounting brackets 1218a and 1218b. In some examples, an antireflective window 1128 may be placed behind the lens mount 1126 and in front of the sample on the substrate to assist in the prevention of back scatter and to protect the optical assembly from contamination. Since lens mount 1126 includes two filters 1126a and 1126b that may be selected based on desired imaging parameters, lens mount 1126 may move laterally along an axis 1204. For example, when filter 1126a is selected, lens mount 1126 may be moved along axis 1204 (e.g., by a motor) in a first direction so that filter 1126a is aligned with lens 1131 and microarray 1115 on the common axis 1201. As another example, when filter 1126b is selected, lens mount 1126 may be moved along axis 1204 in a second direction, opposite of the first direction, so that filter 1126b is aligned with lens 1131 and microarray 1115 on the common axis 1201. Note that in other examples, switchable detection filter 1126 may include more than two filters. In the example illustrated in FIG. 12, lens mount 1126 may be limited to only lateral movement along axis 1204, for example through the use of linear slide 1206. For example, lens mount 1126 may not move along the common axis 1201 to become closer to or father from the microarray 1115. Similarly, switchable detection filters in the lens mount 1126 may not move vertically (e.g., up and down) or rotate. However, in other examples, lens mount 1126 may be a filter wheel or a filter cube. In the example of a filter wheel, lens mount 1126 may be rotated to position a desired filter between lens 1131 and microarray 1115 along the common axis 1201 and may not move laterally or vertically, at least in some examples. In the example of a filter cube, lens mount 1126 may move in a plurality of directions in order to align a desired filter along the common axis 1201 and at a desired distance from each of the lens 1131 and the microarray 1115. The control of the lens mount 1126 will be further described with respect to FIG. 16.

The optical assembly 1150 may optionally include an identification reader such as barcode reader 1208 such that when a cartridge or substrate with an identifier such as a bar code is inserted into the system 1100, the identification included in the identifier may be read and associated with any images taken of a microarray which is affixed to the substrate comprising the identifier.

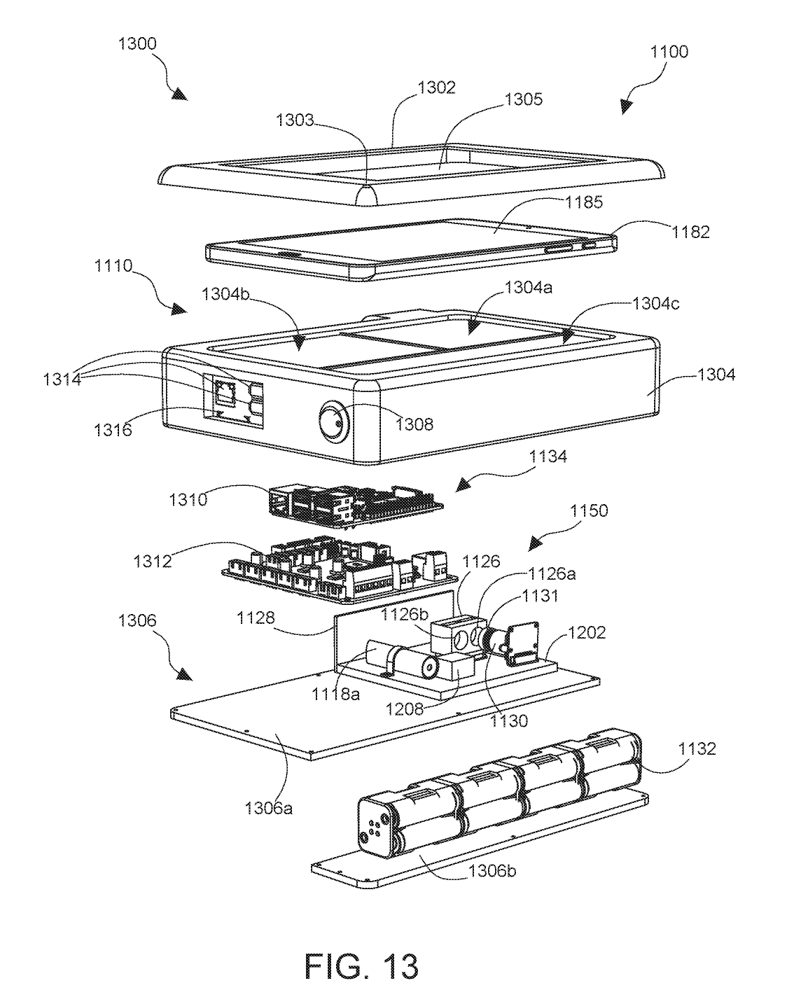

FIG. 13 shows an exploded angled side view 1300 of the biomolecule analysis system 1100 introduced in FIG. 11, including the biomolecule microarray assembly 1110 and the computer 1182. As such, components of FIG. 13 that are the same as components of FIGS. 11 and 12 are numbered the same and may not be reintroduced. In the example shown in FIG. 13, the biomolecule microarray assembly 1110 and the computer 1182 are contained within a single housing 1304 of the biomolecule analysis system 1100. However, in other examples, some components, particularly computer 1182, may not be contained within housing 1304. In the example of FIG. 13, computer 1182 is a tablet, and the display subsystem 1185 is a touch screen that serves as both an input and an output device, providing routines and logic subsystems for running operations including, but not limited to, the user interface, image acquisition control, image quantifier, clinical decision analysis, secure storage manager, external communication manager, and software update manager.

Housing 1304 may be comprised of one or more of metal, plastic, acrylic, or any other suitable material. In particular, housing 1304 may be opaque so that light external to the housing 1304 (e.g., ambient light) is kept out and light internal to housing 1304 (such as generated by first laser 1118a or second laser 1118b, not visible in view 1300, or emitted by the fluorescently labeled microarray 1115, also not visible in view 1300) is kept in. Furthermore, housing 1304 may help protect internal components from harsh environmental conditions, such as dusty conditions, high humidity conditions, and direct sunlight conditions. As one example, the biomolecule analysis system 1100 may be rated for operation in ambient temperatures ranging from 10 to 35° C. and up to 90% non-condensing humidity at an altitude up to 2,500 meters. As another example, the biomolecule analysis system 1100 may be rated for operation in ambient temperatures ranging from 5 to 45° C. and up to 90% non-condensing humidity at an altitude up to 3,000 meters. Thus, housing 1304 may contribute to environmental stability of the biomolecule analysis system 1100.

Housing 1304 may further include a top panel 1302 and a bottom panel 1306. Top panel 1302 may be coupled to housing 1304 via a plurality of fasteners 1303. The fasteners 1303 may be pins, screws, or the like. As shown in FIG. 13, top panel 1302 may include an opening 1305 therein to enable access to the display subsystem 1185 of the computer 1182. For example, top panel 1302 may cover a perimeter of the computer 1182 such that the computer 1182 is partially encased by the top panel and cannot be removed from the housing 1304 without removing top panel 1302. As another example, top panel 1302 may frame the display subsystem 1185 such that the display subsystem 1185 is visible to a user of the biomedical analysis system 1100 and the user can provide input via the display subsystem 1185. Display subsystem 1185 additionally may enable operation of the biomolecule analysis system 1100 in low light conditions.

Furthermore, housing 1304 may include one or more compartments, shown in FIG. 13 as a first compartment 1304a, a second compartment 1304b, and a third compartment 1304c. For example, the first compartment 1304a may house the optical assembly 1150, the second compartment 1304b may house the controller 1134 and/or other electronic components or boards, and the third compartment 1304c may house the battery 1132. Each of the first compartment 1304a, the second compartment 1304b, and the third compartment 1304c may physically isolate the components housed therein, although at least a subset of the components may be electrically and/or communicatively coupled to one another.

As also shown in FIG. 13, in some examples, bottom panel 1306 may be divided into a first bottom panel section 1306a and a second bottom panel section 1306b. Each of first bottom panel section 1306a and second bottom panel section 1306b may be coupled to housing 1304 with one or more fasteners, such as one or more screws. For example, by removing the appropriate screws, one or more of the first bottom panel section 1306a and the second bottom panel section 1306b may be removed to access components of the biomolecule analysis system 1100. As an example, by removing a first set of screws, the first bottom panel section 1306a may be removed to access the first compartment 1304a (e.g., the optical assembly 1150) and/or the second compartment 1304b (e.g., the controller 1134) without accessing the third compartment 1304c (e.g., the battery 1132). As another example, by removing a second set of screws, the second bottom panel section 1306b may be removed to access the third compartment 1304c (e.g., the battery 1132) without accessing the first compartment 1304a (e.g., the optical assembly 1150) and the second compartment 1304b (e.g., the controller 1134). In this way, the bottom panel 1306 may allow selective access to internal components of the biomolecule analysis system 1100, which may help protect the internal components and prevent contamination with dust, dirt, and the like.

Biomolecule analysis system 1100 may further include access to peripherals that connect to the controller 1134 in the electronics space 1304b. For example, it may comprise access to a plurality of ports 1314 and 1316 for wired connection such as an Ethernet, as well as access to memory or program components such as those carried on an SD card or USB drive. Such components as an SD card or USB drive may be used to input information into the system as well collect and store images or analyses performed on captured images including, but not limited to, diagnoses based on the captured images.

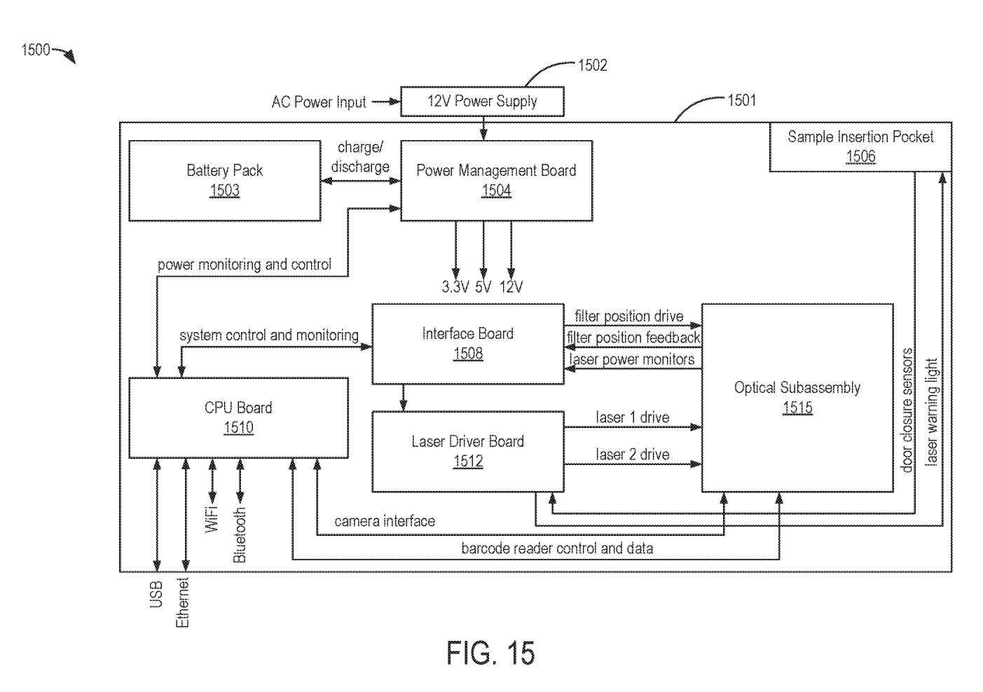

Biomolecule analysis system 1100 may further include a power input 1308. The power input 1308 may enable battery 1132 to be coupled to an electrical socket, such as via an AC power adapter, while battery 1132 remains within housing 1304. Thus, the power input 1308 provides access to battery 1132 through housing 1304. Power management of biomolecule analysis system 1100 will be further described with respect to FIG. 15.

The controller 1134 is shown including a central processing unit (CPU) 1310. As one example, the CPU 1310 may be a Raspberry Pi CPU, which may help reduce costs of the biomolecule analysis system 1100. The CPU 1310 may connect to one or more electronics/connector boards 1312 which perform a variety of functions including, but not limited to, processing, connecting and data handling between two or more components of the microarray assembly. As a non-limiting example, the CPU 1310 may have at least a 1.4 GHz 64-bit quad-core with an ARM Cortex-A53 microarchitecture. Depending on the computing needs of the system, CPUs such as those built to ARM Cortex-A53 specifications may be exchanged for CPUs with additional processing capabilities such as the Cortex-A55 or Cortex A7X, or by moving to a processor with more cores, or both. In systems with lower processing needs, CPUs with lower processing capabilities such as the Cortex A3X family and the like may be used. A configuration of on-board electronics of the biomolecule analysis system 1100 will be further described with respect to FIG. 15. Furthermore, the CPU 1310 may be operable in a range of environmental conditions, such as a range of temperature conditions (e.g., 5 to 45° C.), high humidity conditions (e.g., 90% non-condensing humidity), and high altitude conditions (e.g., 2,500-3,000 meters). As described above, the housing 1304 may also contribute to the environmental stability of the CPU 1310. For example, the CPU 1310 may be operable in more extreme environmental conditions (e.g., both higher and lower temperatures, dustier conditions, more humid conditions, etc.) while enclosed within housing 1304 compared to the CPU 1310 alone.

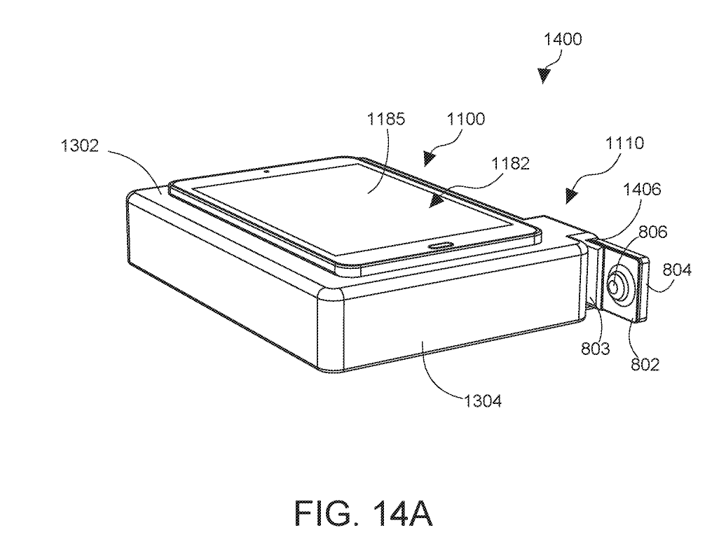

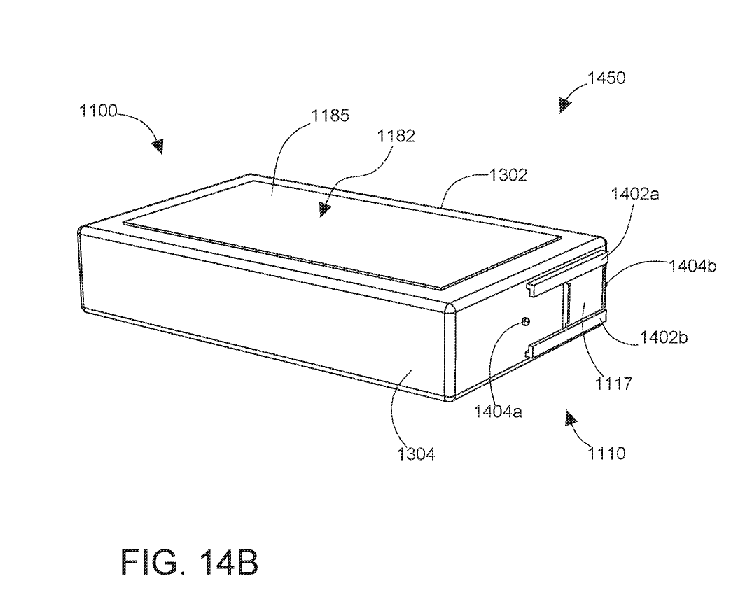

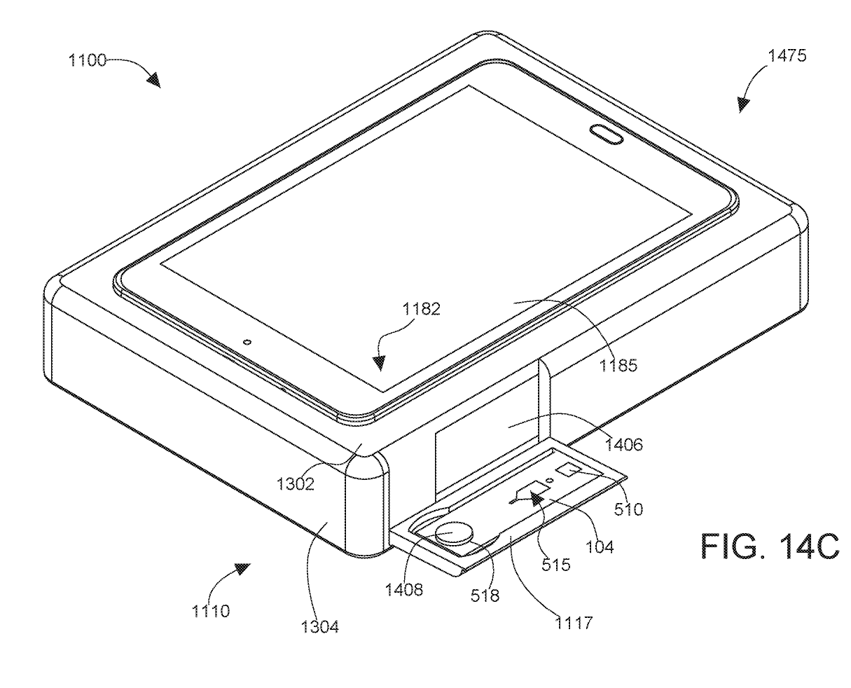

FIGS. 14A-14C show external views of the biomolecule analysis system 1100 introduced in FIG. 11. As such, components of FIG. 14 that function the same as components of FIGS. 11-13 are numbered the same and may not be reintroduced. Specifically, FIG. 14A shows a first view 1400 where the door 1117 is in an open position, and FIG. 14B shows a second view 1450 where the door 1117 is in a closed position. FIG. 14C shows a view 1475 of an alternative configuration where an embodiment of the door 1117 is in the open position.

As shown in FIG. 14A, the door 1117 may be slidable between the open position and the closed position. With the door 1117 in the open position, a sample insertion pocket 1406 is uncovered so that the cartridge 800 may be inserted into the biomolecule analysis system 1100.

With the door 1117 in the closed position as shown in FIG. 14B, the sample insertion pocket 1406 is inaccessible. For example, the door 1117 may be closed after the cartridge 800 is inserted into the biomolecule analysis system 1100 (via the sample insertion pocket 1406 while the door 1117 is open) to enable imaging to commence. As further described herein, the first laser 1118a and second laser 1118b (not visible in FIGS. 14A-14C) may only be activated once insertion of the cartridge 800 is confirmed and the door 1117 is confirmed to be in a closed position. For example, one or more door closure sensors may be communicatively coupled to the controller 1134 (not visible in FIGS. 14A-14C) to indicate when the door 1117 is closed. As another example, the door 1117 may be closed after the cartridge 800 is removed from the biomolecule analysis system 1100 in order to prevent dirt, dust, and the like from accumulating within the sample insertion pocket 1406. Thus, the door 1117 enables selective access to the sample insertion pocket 1406. In some embodiments, sliding the door 1117 to the open position shown may include pushing the door 1117 along the tracks 1402a and 1402b until the door 1117 contacts a first mechanical stop 1404a. The first mechanical stop 1404a may prevent the door 1117 from sliding beyond the open position and off of the tracks 1402a and 1402b.

However, in other examples, the door 1117 may be a hinged door instead of a slidable door, such as shown in FIG. 14C. In such an example, opening the door 1117 may include flipping down the door 1117, which pivots outward from the housing 1304 on a pivot axis located at the bottom of door 1117, and closing the door may include flipping up the door 1117 so that door 1117 becomes flush with housing 1304. In some embodiments the door 1117 may include a latch 1408 or other mechanism to hold the substrate 104 in place, for example using optional opening 518 in the substrate. In further embodiments, the door 1117 may have a lip to hold the substrate 104 in place such that when the door 1117 is closed the substrate 104 is positioned for the microassay assembly to analyze the microarray. In other embodiments, cartridge 800 may be placed through opening 1406. While the insertion pocket 1406 is shown as rectangular, other geometries of insertion pocket 1406 are also possible, such as square, oval, circular, and triangular.

As one example, the door 1117 may be manually moved between the open position and the closed position by a user of the biomolecule analysis system 1100. As another example, movement of the door 1117 may be controlled by the controller 1134, such as via an electric motor included in one or more of the tracks 1402a and 1402b (e.g., in the example shown in FIG. 14A) or in the housing 1304 (e.g., in the example shown in FIG. 14C). Furthermore, in some examples, the door 1117 may resist movement due to friction between the door 1117 and the tracks 1402a and 1402b. In other examples, additionally or alternatively, one or more latches, such as spring-loaded latches, may hold the door 1117 in the open position and/or the closed position. Thus, an application of force by the user (or the electric motor, if included) that overcomes a force of the friction and/or a force of the one or more latches, if included, may move the door 1117 between the open position shown in FIG. 14A and FIG. 14C and the closed position shown in FIG. 14B.

Next, FIG. 15 shows a block diagram 1500 of exemplary electronics of a biomolecule microarray assembly. Specifically, the electronics represented in block diagram 1500 may be included in biomolecule microarray assembly 1110 shown in FIGS. 11-14C, although it should be understood that similar electronics may be included in biomolecule microarray assembly 1010 shown in FIG. 10. The block diagram 1500 may be part of a larger system such as the system shown in FIG. 13 comprising the interconnection of several components including one or more network interfaces such as a USB, Ethernet, Wi-Fi, GSM/LTE or Bluetooth mechanism, a display such as the computer 1182 which may itself contain a central processing unit and a memory, and the on-board electronics of the microarray assembly 1110.

Block diagram 1500 includes on-board electronics 1501, which include a battery pack 1503, a power management board 1504, a sample insertion pocket 1506, an interface board 1508, a CPU board 1510, a laser driver board 1512, and an optical subassembly 1514. Components of the on-board electronics 1501 may communicate with external electronics. For example, power management board 1504 may receive power from a 12 V power supply 1502, which may further receive input from an AC power adapter. As another example, CPU board 1510 may communicate with other computing devices (e.g., computer 1182 shown in FIGS. 11-14B) via wired or wireless connections. Furthermore, all or a subset of the on-board electronics 1501 may comprise a controller (e.g., controller 1134 shown in FIGS. 11 and 13).

The power management board 1504 may regulate the charging and discharging of the battery pack 1503 in combination power monitoring and control data communicated with the CPU board 1510. For example, the power management board 1504 may receive power monitoring and control commands from the CPU board 1510 and transmit feedback regarding a state of charge of the battery pack 1503, an output voltage supplied to various components of the biomolecule microarray assembly, etc. to the CPU board 1510. When external power is available, the battery pack 1503 may be charged. When external power is unavailable or interrupted, the biomolecule microarray assembly may continue to function using the battery pack 1503. The battery pack 1503 may be any suitable battery, such as lithium-ion, lead-acid, solid polymer electrolyte, molten salt, etc. As an example, the battery pack 1503 may comprise one or more NiMH rechargeable 10.6 V batteries having a 4000 mA-hours capacity, as one example. The power management board 1504 may supply power to components of the biomolecule microarray assembly, including components of the on-board electronics 1501, at a variety of voltages, including 3.3 V, 5 V, and 12 V.

The microarray assembly may further include memory (not shown) capable of storing instructions to be executed by the central processing unit. Such memory generally comprises a random access memory (“RAM”) and permanent non-transitory mass storage device, such as a hard disk drive or solid-state drive. Memory stores an operating system, as well as other components such as routines to manipulate the optical subassembly 1514, laser driver board 1512, interface board 1508, and power management board 1504. These and other software components may be loaded into memory of the system using a drive mechanism (not shown) associated with a non-transitory computer-readable medium, such as a floppy disc, tape, DVD/CD-ROM drive, memory card, or the like. In some embodiments, biomolecule analysis system 1100 may communicate with databases via a network interface such as the Wi-Fi or Bluetooth system shown, a storage area network (“SAN”), a high-speed serial bus, and/or via the other suitable communication technology. In some embodiments, the database may comprise one or more storage resources provisioned from a “cloud storage” provider, for example, Amazon Simple Storage Service (“Amazon S3”), provided by Amazon.com, Inc. of Seattle, Wash., Google Cloud Storage, provided by Google, Inc. of Mountain View, Calif., and the like.

The CPU board 1510 may be a printed circuit board that contains the CPU (e.g., CPU 1310 shown in FIG. 13). In addition to communicating with the power management board 1504, the CPU board 1510 may send and receive signals with the interface board 1508 and the optical subassembly 1514. For example, the CPU board 1510 may send system control signals to the interface board 1508 and receive system monitoring feedback signals from the interface board 1508, which the CPU board 1510 may use to adjust the system control signals sent to the interface board 1508. The interface board 1508 may in turn send and/or receive signals with the laser driver board 1512 and the optical subassembly 1514. The optical subassembly 1514 may include electronics for one or more lasers (e.g., first laser 1118a and second laser 1118b shown in FIGS. 11-13), a camera (e.g., camera 1130 shown in FIGS. 11-13), and a filter (e.g., a filter in lens mount 1126 shown in FIGS. 11-13), while the laser driver board 1512 may control operation of the laser.

For example, the interface board 1508 may adjust a filter position (e.g., filter position drive) based on commands received from the CPU board 1510 and filter position feedback received from the optical subassembly 1514. Furthermore, the interface board 1508 may transmit command signals to the laser driver board 1512 so that the laser driver board 1512 may turn on the appropriate laser line in the optical subassembly 1514 (e.g., laser 1 drive or laser 2 drive) at the appropriate timing, frequency, and laser power. For example, the laser driver board 1512 may only turn on the laser when a door of the biomolecule microarray assembly is closed (e.g., door 1117 shown in FIGS. 11 and 14A-14B), determined based on feedback from door closure sensors of the sample insertion pocket 1506. Furthermore, the laser driver board 1512 may turn on a laser warning light at the sample insertion pocket 1506 in order to alert a user of the biomolecule microarray assembly that the laser is turned on. Further still, the interface board 1508 may monitor the laser power based on feedback signals received from the optical subassembly 1514.

As another example, the CPU board 1510 may send and receive signals with the optical subassembly 1514 regarding the camera interface and barcode reader control and data. For example, the optical subassembly 1514 may include a barcode reader for identifying a sample inserted into the sample insertion pocket 1506. The sample may be included on a barcoded substrate (e.g., flat substrate 500 as shown in FIG. 5 and/or flat substrate 1112 shown in FIGS. 11, 12, and 14A) or a barcoded chip (e.g., chip 1012 shown in FIG. 10). Thus, the sample insertion pocket 1506 may correspond to the electronic components of the sample insertion pocket 1406 shown in FIGS. 14A and 14C. The optical subassembly 1514 may read the barcode, such as with the camera, and transmit the barcode data to the CPU board 1510. The CPU board 1510 may also control operation of the camera via the optical subassembly 1514, such as by adjusting one or more camera parameters, including shutter closure timing, shutter speed, focus, zoom, aperture, gain etc. Similarly, the optical subassembly 1514 may transmit data obtained by the camera to the CPU board 1510. In this way, the optical subassembly 1514 may be controlled via signals received from the CPU board 1510, the interface board 1508, and the laser driver board 1512, with the CPU board 1510 providing system command signals to the interface board 1508 (which in turn provides commands to the laser driver board 1512) as well as the power management board 1504, to capture images of a microarray assay inserted in the sample insertion pocket 1506 and store the resulting images.

FIG. 16 shows a flow chart of a method 1600 for imaging a microarray (e.g., microarray 1015 shown in FIG. 10 or microarray 1115 shown in FIG. 11) and analyzing images taken of the array to determine an identity and/or quantity of biomolecules from a patient sample that are bound to the microarray. For example, the microarray may comprise a plurality of binding locations (e.g., analyte capture locations 1016 shown in FIG. 10 or analyte capture locations 1116 shown in FIG. 11) for binding biomolecules of the patient-derived sample to the microarray, such as biomarkers for diseases. The microarray may be positioned on a nitrocellulose film (e.g., nitrocellulose film 1014 shown in FIG. 10 or nitrocellulose film 1114 shown in FIG. 11), the nitrocellulose film forming a coating on substrate, such as a microarray chip (e.g., microarray chip 1012 shown in FIG. 10 or the flat substrate 1112 shown in FIGS. 11, 12, and 14A). Once the biomolecules from the sample have been bound to the analyte capture locations and the microarray processed (such as according to the example method of FIG. 9), the microarray may be inserted into a biomolecule microarray assembly of a biomolecule analysis system (e.g., biomolecule microarray assembly 1010 of biomolecule analysis system 1000 shown in FIG. 10 or biomolecule microarray assembly 1110 of biomolecule analysis system 1100 shown in FIG. 11) for imaging. A laser (e.g., laser 1018 shown in FIG. 10 or laser 1118a or 1118b shown in FIG. 11) may be powered on to excite fluorescent labels chemically bound to the biomarkers. Specifically, a controller (e.g., controller 1034 shown in FIG. 10 or controller 1134 shown in FIG. 11) may be in communication with the laser for adjusting operation of the laser, such as according to the electronics block diagram of FIG. 15. In response to excitation light from the laser, the fluorescent labels may emit light, which may be captured in an image by a camera (e.g., camera 1030 shown in FIG. 10 or camera 1130 shown in FIG. 11). The controller may send signals to the camera for capturing images of the array. In particular, in contrast to laser scanning techniques, including a camera for digital image capture may reduce acquisition time. For example, the acquisition of signal from the complete microarray may be obtained in 1 second or less. In this way, the controller may include computer readable instructions for executing a method, such as method 1600. As such, method 1600 may be executed by the controller based on input from a user, such as via a computer of the biomolecule analysis system (e.g., computer 1082 shown in FIG. 10 or computer 1182 shown in FIG. 11).

In some examples, the images acquired by the biomolecule analysis system may be analyzed by the controller of the biomolecule microarray assembly. However, in other examples, the computer of the biomolecule analysis system may be used to analyze the acquired images. In still other examples, a computer not included in the biomolecule analysis system may be used to analyze the acquired images, for example, the biomolecule analysis system may send the images to a remote computer such as in the cloud using, for example, Wi-Fi or GSM/LTE communication.

Method 1600 begins at 1602 and includes receiving user input. As described above, user input may be received by the controller from a touchscreen or button pad included on the microarray assembly. However, in other examples the user input may be received from the computer, which may be in communication with the controller of the microarray assembly via either a wired or wireless connection. The user input may include commands for one or more of powering on or off the laser, taking a picture with the camera, and selecting one or more filters for filtering light that is captured by the camera. In some examples, such as where the biomolecule analysis system is configured to analyze acquired images, the user input may additionally include selecting a regional epidemiological context for the imaging. For example, the user may select which analyses results are run based on conditions in the region in which the test is applied, such as based on which diseases, contaminants, etc. are endemic to the region. Such a selection may streamline the analysis process and improve the accuracy. In other examples, this selection may be overridden and a general analysis applied.

At 1604, method 1600 includes determining if the microarray has been inserted into the microarray assembly. In one example, it may be determined that the flat substrate is inserted if a door (e.g., door 1117 shown in FIGS. 11 and 14A-14C) through which the microarray on its substrate is inserted into the assembly is closed. The position of the door may be determined based on a signals received from door position sensors, such as a voltage and/or current output from a circuit included on an interior surface of the door. Thus, the current in the circuit may change depending on the position of the door. Specifically the current in the circuit may increase when the door is in a closed position relative to when the door is in an open position. In another example, it may be determined if the flat substrate has been inserted based on a state of a mechanical or electrical switch of the assembly. In some examples, a bottom of the substrate and/or any covers or sheaths may interface with the switch of the assembly to transform the state of the switch. Thus, the state of the switch may change depending on whether the substrate and/or cover is inserted into the assembly.

Furthermore, if the flat substrate includes a barcode or RFID, determining if the microarray is inserted into the microarray assembly may additionally or alternatively include reading the barcode or RFID to positively identify the sample. However, in other examples, the user may manually enter the sample identification, such as an alphanumeric patient identifier, using a keypad or touchscreen. In such examples, the user may be further prompted to confirm that the microarray is inserted into the microarray assembly.

If it is determined that the door is open and/or that the substrate with the microarray is not inserted into the assembly, then method 1600 proceeds to 1606 and includes not powering on the laser. In this way, the laser may only be powered on when the substrate with the microarray is inserted into the assembly and the door of the assembly is closed to prevent light from escaping or entering the assembly during imaging of the microarray. Method 1600 may then end.

However, if it is determined that the door is closed and/or that the substrate with the microarray is inserted into the assembly, method 1600 proceeds to 1608, which optionally includes adjusting a detection filter position based on a desired detection wavelength. For example, if the biomolecule microarray assembly includes a switchable detection filter having more than one filter position, such as lens mount 1126 shown in FIGS. 11-13, the controller may adjust the detection filter position so that the filter corresponding to the desired detection wavelength is positioned between the microarray and the camera. The desired detection wavelength may be determined based on an emission spectrum the fluorophore(s) used. For example, the fluorophore(s) used may be input by the user. In another example, the user may directly select the filter position.

At 1610, method 1600 includes turning on the laser for a duration to excite fluorescent labels in the microarray. Furthermore, where the laser includes more than one excitation source, turning on the laser may further include activating a desired excitation source, the desired excitation source selected based on the input received from the user (e.g., at 1602). In some examples, the duration may be an amount of time. The duration may be a pre-set value stored in the memory of the controller. However, in other examples the duration may be adjustable based on input from the user. Thus, the user may adjust the amount of time that the laser is powered on. In still further examples, the duration may be based on the configuration of the assembly, such as the wavelength of light produced by the laser, intensity of the light beam produced by the laser, a distance between the laser and the array, type or excitation wavelength of the fluorophore(s) used in the probe molecules to fluorescently tag the target biomolecules, resolution and/or sensitivity of the camera, concentration or amount of biomarkers bound and/or fluorescently tagged on the microarray, etc. Furthermore, the laser may be turned on at a desired laser power. In some examples, the desired laser power may be a pre-set value stored in the memory of the controller. Further, the desired laser power may vary based on the duration. For example, the desired laser power may increase as the duration decreases. In another example, the desired laser power may decrease as the duration increases in order to avoid photobleaching of the fluorophore. Additionally or alternatively, the laser power may be adjusted from the pre-set value based on user input.

In some examples, the light emitted by the laser may be directly incident on the fluorophores without passing through or being directed by additional optical elements (e.g., filters or mirrors), such as in the example system shown in FIG. 11. In other examples, the light emitted by the laser may be directed to one or more optical elements before reaching the microarray, such as in the example system shown in FIG. 10.

After exciting the fluorescent labels at 1610, method 1600 may then proceed to 1614, which includes capturing an image of light emitted by the fluorescent labels in the microarray with the camera. Specifically, the controller may send signals to the camera for capturing an image of the microarray. In some examples, the camera may capture more than one image. Camera settings may in some examples be pre-set, or in other examples, may be adjustable based on user input. The camera settings may include a number of images to capture, exposure duration, focal length of a lens (e.g., lens 1031 shown in FIG. 10 or lens 1131 shown in FIG. 11), an f-stop setting of the lens, camera gain, frame transfer speed, timing of the image acquisition relative to powering on of the laser, etc.

Additionally, digital information corresponding to the image may be acquired at 1614. Specifically, information corresponding to a location of each of a number of spots (e.g., spots 1016 shown in FIG. 10 or spots 1116 shown in FIG. 11) of the microarray as well as an intensity of each pixel may be acquired at 1614. For example, a spot-by-spot delimited list of each spot's column and row placement in the assay, its name, identity, etc. may be acquired at 1614. Further, the method 1600 at 1614 may comprise identifying images of the array that are rotated, inverted, and/or reversed. For example, the method 1600 at 1614 may comprise determining an angle of rotation of the image of the assay, which may be any angle between 0 and 360 degrees, and adjusting the orientation of the image so that it aligns with pre-set orientation conditions. Thus, at 1614, the image acquired may be one or more of rotated and/or flipped (e.g., reflected across an axis) so that the orientation of the image acquired at 1614 matches a pre-set orientation. For example, during each successive iteration of method 1600 (e.g., during multiple image acquisitions), each of the images of the one or more assays may be adjusted so that their orientation relative to one another is approximately the same.

One or more of the images taken at 1614 may then be stored at 1616. In some examples, the images may be stored in non-transitory memory of the assembly, such as on the controller and/or a storage device (e.g., memory chip 1033 shown in FIG. 10 or memory chip 1133 shown in FIG. 11). Additionally or alternatively, the method at 1616 may include transferring the images taken at 1614 to the computer for storage therein. In further examples, the images may be stored in non-transitory memory of any suitable device for storing digital images, such as a memory chip or card, flash drive, etc. In still further examples, the images may be transferred via a direct electrical connection between the microarray assembly and an external source and/or may be transferred via the storage device. For example, the memory chip may be removed from the microarray assembly and inserted into an external source so that the images contained on the memory chip may be uploaded to the external source. In yet further examples, the images may be transferred via a wireless connection such as sub-GHZ, ZigBee, Bluetooth, GSM/LTE, passive RF, or Wi-Fi or other electromagnetic wave frequency suitable for wirelessly transmitting data packets containing image data. Furthermore, information corresponding to the location of each of the spots of the array may be stored at 1616. For example, the spot-by-spot delimited list of each spot's column and row placement in the assay, its name, identification, etc. may be stored at 1616.

Thus, after storing the images at 1616, the image capturing of the microarray may be complete. Said another way, the steps in method 1600 up to and/or including 1616 may be executed to capture and store an image of the microarray. In examples where the microarray assembly is configured as an image capturing device only, method 1600 may end after storing the images at 1616. However, in examples where the microarray assembly is configured as both an image capturing device and an image analysis device, the controller of the microarray assembly may perform the analysis of the one or more images stored at 1616 to identify the presence and/or quantity of biomarkers bound to the microarray.

Thus, in some examples, method 1600 may optionally continue to 1618 and 1620, which may be executed to perform the analysis of the one or more images stored at 1616. It should be appreciated that in examples where the microarray assembly is configured as an image capturing device only, 1618 and 1620 may be executed by a source external to the microarray assembly. Specifically, computer readable instructions may be stored in non-transitory memory of the external source, where the instructions may be executable by a controller of the external source to perform a method such as 1618 and 1620 of method 1600.

Thus, at 1610, method 1600 optionally includes determining the spot locations after storing the images at 1616. Specifically, the spot location analysis performed at 1618 may include identifying the location and content, i.e. binding ligand of each of the spots on the assay, including spots in which fluorescence is present (e.g., due to fluorescently labeled biomarkers) and spots that are not fluorescing (e.g., due to an absence of fluorescently labeled biomarkers). The spot location determination process may begin by acquiring the spot-by-spot delimited list of each spot's column and row placement in the microarray, its name, identification, etc. Information about the spot location may be stored during image acquisition at 1614 and image storage at 1616, as described above. Based on an origin, a series of field points where each spot is expected to be located may be determined.

Identification of the origin of the microarray can be accomplished using an image recognition method. In one example of an image recognition method, a set of fiducial spots are printed onto a first row of the binding locations of the microarray to serve as an image recognition and alignment feature. The fiducial spots may be a pattern of spots separated by blanks, dilution series, or any other features that fluoresce. An image may be created of a first row of spots, and that portion of the image may be saved as a recognition template. This template can be re-used for any microarray that has been printed with the same pattern of fiducials appearing in the first row. With this template as a guide, the corresponding pattern in each image may be located and used in that subset of the image to identify the spot located at the origin, which may be the first row and column in the array. Once the origin is identified, the projected field points for all spots are calculated based on their coordinates derived from the image information stored at 1616. If no recognition spots have been printed on the microarray, the first row of spots can serve as a surrogate recognition template for its own microarray image.

Subsequently, a series of measurements may be performed to identify the actual location of each spot. Using the calculated field points, a centroid of intensity located in a region surrounding each field point may be located. The centroid may be equivalent to the first moment of the spot region, defined as:

$$C = \frac{{Σ}_{p = 1}^{N} {I}_{p} ( {\overline{X}}_{p} + {\overline{Y}}_{p} )}{{Σ}_{p = 1}^{N} {I}_{p}}$$

where Iₚ is the pixel intensity at the pixel p, X̅ₚ and Y̅ₚ are the distance vectors to the pixel p from an arbitrary reference location, and N is the total number of pixels in the region. Once the centroid is located, the field point is re-centered on the spot centroid. With this new location as an origin, the method may include locating inflection points in the gradient of intensity along vertical and horizontal lines through the region. These points may define the lines of steepest descent in the gradient of the signal, points that are directly related to the spot boundaries. At these inflection points, the gradient will be maximized, and the second derivative of the intensity with respect to the radial coordinate will be zero, such as according to the equation:

$$\frac{{\partial }^{2}}{\partial {r}^{2}} I ( r , ∅ ) = 0$$

Averaging the two inflection point locations along each axis provides the coordinates of the actual spot center. With this point as the new center, the method may then include searching for a circle of signal whose intensity is bounded by adjustable lower and upper limits relative to the local background. These limits define the sensitivity of the location algorithm relative to the background. A single sensitivity setting may be used for all images produced by a particular combination of spot diameter, print pitch, and assay protocol. The method may include keeping a running inventory of the calculated spot center and the actual spot centers during the microarray spot location routine. From the differences of these sets, adjustments for possible image rotation and drifts during the printing may be made. As such, the need to rotate or stretch a “grid” over the microarray to account for deviations in spot locations from their projected centers may be reduced or eliminated.

After determining the location of each of the spots at 1618, method 1600 may then optionally proceed to 1620, which includes performing a wavelength analysis to determine the presence and/or levels of biomarkers at each spot. In some examples, only one wavelength of emitted light may be used for analysis at 1620. Specifically, fluorescent tags of only one wavelength of light may be used to tag the biomarkers (e.g., target biomolecules). Thus, based on the emission intensities of the wavelength of light used to tag the biomarkers, one or more of an amount, concentration, and/or level of the biomarkers at each spot may be inferred. However, in other examples, the analysis at 1620 may include multiplexing and analysis of two or more wavelengths of light, corresponding to two or more target biomolecules fluorescently tagged using different labels. Put more simply, in examples where two fluorescent labels are used that have different emission spectra, quantification of the intensity of each wavelength corresponding to the two or more labels may be used to determine biomarker expression levels. For example, the image acquisition may be performed iteratively using multiple excitation sources and detections filters, such as by repeating some or all of method 1600. In other examples, multiple different fluorescent labels may be excited and imaged simultaneously. In some examples, the light emitted by the different fluorescent tags may be sufficiently separated in wavelength to discern the different tags. However, in some examples, various statistical algorithms or data visualization software programs, such as spectral unmixing, may be used to analyze the relative intensities of different wavelengths of electromagnetic waves emitted by the array and captured by the camera. In some examples, the analysis at 1620 may be used to diagnose an infectious disease and/or non-infectious disease. Thus, the method 1600 at 1620 may include generating results from the analysis, where the results may comprise an estimation of biomarker presence and/or levels in the microarray. As an example, based on fluorescence measured at one or more spots in the microarray, it may be determined that the patient sample includes biomarkers for positive diagnosis of a disease or condition.

In some examples, method 1600 may end after determining the presence and/or levels of the biomarkers bound to the microarray. However, in other examples, the method 1600 may continue from 1620 to 1622, which includes displaying the results via a display screen. Thus, estimations of biomarker levels (e.g., antibody, protein, antigen, DNA, and/or gene concentrations), derived from the analysis at 1620, may be presented to the user via the display screen at 1622. Alternatively, a presence of the biomarkers may be presented to the user via the display screen without additional information concerning the levels. Further, a disease diagnosis may additionally be displayed to the user at 1612 based on the positively identified biomarkers and/or the determined biomarker levels. Method 1600 then ends.

In some embodiments, the nitrocellulose porous strips, substrates, reagents, and biomolecule analysis system may be packaged and sold as a kit. The kits may comprise one or more nitrocellulose porous strips alone or extruded onto a substrate, where the substrate is independent or part of a larger system such as a cartridge, with or without microarrays of binding ligands, various reagents, a user interface, a biomolecule microarray assembly, optical assembly, molecular probes, fluorescent labels, and labeled probes. The kits may comprise one or more of the components disclosed herein, information which informs the user of the kit, by words, pictures, electronic interface and the like, how to use the kit in the identification of biomarkers in a biological sample and/or the use of the components of the kit in the diagnosis of a disease or condition. In another example, kits may comprise polymer mixtures, solvents, and devices for the manufacture and production of polymeric strips. The kit may further include a sample collection device and disinfectant wipes.

The techniques and procedures described herein for the biomolecule microarray assembly as well as the biomolecule microarray assembly system may be implemented via logic distributed in one or more computing devices. The particular distribution and choice of logic may vary according to implementation. In some embodiments, a plurality of remotely distributed subscriber computers such as a plurality of biomolecule microarray assemblies are connected to the internet. Requests from the plurality of remotely distributed subscriber computers are distributed through elastic load balancing, i.e., automatic scaling of request-handling capacity in response to incoming application traffic. Such requests may be concurrent, consecutive, or some combination thereof. The requests are then sent to a plurality of application servers, 1-x. The number of application servers may increase or decrease x times depending on the load received.

References to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to a single one or multiple ones. Additionally, the words “herein,” “above,” “below” and words of similar import, when used in this Application, refer to this Application as a whole and not to any particular portions of this Application. When the claims use the word “or” in reference to a list of two or more items, that word covers all of the following interpretations of the word: any of the items in the list, all of the items in the list and any combination of the items in the list, unless expressly limited to one or the other. “Logic” refers to machine memory circuits, non-transitory machine readable media, and/or circuitry which by way of its material and/or material-energy configuration comprises control and/or procedural signals, and/or settings and values (such as resistance, impedance, capacitance, inductance, current/voltage ratings, etc.), that may be applied to influence the operation of a device. Magnetic media, electronic circuits, electrical and optical memory (both volatile and nonvolatile), and firmware are examples of logic. Logic specifically excludes pure signals or software per se (however does not exclude machine memories comprising software and thereby forming configurations of matter). Those skilled in the art will appreciate that logic may be distributed throughout one or more devices, and/or may be comprised of combinations memory, media, processing circuits and controllers, other circuits, and so on. Therefore, in the interest of clarity and correctness logic may not always be distinctly illustrated in drawings of devices and systems, although it is inherently present therein. The techniques and procedures described herein may be implemented via logic distributed in one or more computing devices. The particular distribution and choice of logic will vary according to implementation.

Those having skill in the art will appreciate that there are various logic implementations by which processes and/or systems described herein can be effected (e.g., hardware, software, and/or firmware), and that the preferred vehicle will vary with the context in which the processes are deployed. “Software” refers to logic that may be readily readapted to different purposes (e.g. read/write volatile or nonvolatile memory or media). “Firmware” refers to logic embodied as read-only memories and/or media. Hardware refers to logic embodied as analog and/or digital circuits. If an implementer determines that speed and accuracy are paramount, the implementer may opt for a hardware and/or firmware vehicle; alternatively, if flexibility is paramount, the implementer may opt for a solely software implementation; or, yet again alternatively, the implementer may opt for some combination of hardware, software, and/or firmware. Hence, there are several possible vehicles by which the processes described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary.

Those skilled in the art will recognize that optical aspects of implementations may involve optically-oriented hardware, software, and or firmware. The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood as notorious by those within the art that each function and/or operation within such block diagrams, flowcharts, or examples can be implemented, individually and/or collectively, by a wide range of hardware, software, firmware, or virtually any combination thereof. Several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in standard integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies equally regardless of the particular type of signal bearing media used to actually carry out the distribution. Examples of a signal bearing media include, but are not limited to, the following: recordable type media such as floppy disks, hard disk drives, CD ROMs, digital tape, flash drives, SD cards, solid state fixed or removable storage, and computer memory.

In a general sense, those skilled in the art will recognize that the various aspects described herein which can be implemented, individually and/or collectively, by a wide range of hardware, software, firmware, or any combination thereof can be viewed as being composed of various types of “circuitry.” Consequently, as used herein “circuitry” includes, but is not limited to, electrical circuitry having at least one discrete electrical circuit, electrical circuitry having at least one integrated circuit, electrical circuitry having at least one Application specific integrated circuit, circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), circuitry forming a memory device (e.g., forms of random access memory), and/or circuitry forming a communications device (e.g., a modem, communications switch, or optical-electrical equipment).

Those skilled in the art will recognize that it is common within the art to describe devices and/or processes in the fashion set forth herein, and thereafter use standard engineering practices to integrate such described devices and/or processes into larger systems. That is, at least a portion of the devices and/or processes described herein can be integrated into a network processing system via a reasonable amount of experimentation.

The foregoing described aspects depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality.

These and other aspects of the present invention will be further appreciated upon consideration of the following Examples, which are intended to illustrate certain particular embodiments of the invention but are not intended to limit its scope, as defined by the claims.

### EXAMPLES

### Example 1

### Microarray Validation

For validation studies, a plurality of existing well known and well-characterized antigens representing nine pathogens are examined. Antigens (133 in total) corresponding to nine pathogens are printed, in duplicate, on nitrocellulose-coated glass ONCYTE® AVID chips in the form of a microarray. Additionally, human IgG and IgM and buffer are printed as positive and negative controls respectively. Imaging fiducials are printed for reference. The microarray is printed three times in three separate print runs to normalize results against printing variation. The 133 antigens and positive controls are printed at concentrations of 0.3, 0.1, 0.03, 0.01, 0.003, 0.001 mg/mL (˜1 nL per spot) in PBS/0.001% Tween 20, an optimized buffer. The trace of detergent both reduces non-specific binding of protein to printing pins and other solid surfaces and increases fluidity and spot size.

Each of the three individual microarrays of 133 antigens is probed with standard, well-characterized serum samples of known origin and disease-causing pathogens. Multiple samples for each of the nine represented pathogens is used, and only one sample (of known origin) is used at a time for probing each microarray. The serum samples are diluted 1:100 in protein array blocking buffer (Super G, Grace Bio-Labs, Bend, Oreg.) and pre-incubated at room temperature for 30 mins. Concurrently, the microarrays are rehydrated in blocking buffer for 30 minutes. Blocking buffer is removed, and each microarray is probed with pre-incubated serum samples in Proplate® chambers. Each microarray is incubated overnight at 4° C. with gentle agitation. After appropriate washing with buffer to remove any unbound biomolecules from the serum samples, the microarrays are incubated with mixtures of secondary antibodies (at 1/200 dilution) directly conjugated to Alexa Fluors® with non-overlapping emission spectra as follows: anti-IgG conjugated to Alexa Fluor® 647, and anti-IgM conjugated to Alexa Fluor® 790. The antibodies are purchased from Jackson ImmunoResearch and conjugated to the Alexa Fluors® in-house. After incubation, the microarrays are washed to remove any unbound secondary antibodies and air dried by centrifugation.

IgM and IgG response sensitivity is determined for each of the 133 antigens. The microarrays are imaged using a camera-based fluorescence imaging system. For each type of Alexa Fluor®, imaging system settings are set at a gain of 50 and the exposure time (in milliseconds) is adjusted to achieve saturation for ˜10% of the microarray spots. A GenePix Array List (gal), which is a map of each spot location developed as part of the printing process, is used by the imaging system software to decode the identification of each printed spot. The intensity of the fluorescent signal for each antigen spotted is recorded and stored in a data table for subsequent analysis. Signal intensities are corrected for spot-specific background and normalized using inter- and intra-array standard control spots (human IgG and IgM) to account for any printing or secondary antibody experimental variation.

Cross-reactivity with other antigens is determined for each of the 133 antigens. The precise location of each antigen for each pathogen is known from the printing schema. As each array is probed with only one sample (of known origin) at a time, if no cross-reactivity is present, only the antigens associated with that sample show fluorescent signals. If additional off-target fluorescent signals are present, the nature of the potential cross-reactivity is investigated, including if there is prior literature to support this finding from another technology (e.g., ELISA). The cross-reactive antigen is flagged as potentially undesirable for final inclusion in the microarray, and other options are investigated (e.g., blocking mechanisms and varying antigen concentration) to potentially remove the cross-reactivity.

With the full dataset of arrays probed with each sample, a cluster analysis is performed to down-select and prioritize the antigens having the highest sensitivity with the least cross-reactivity to non-target pathogens to move to the next microarray version.

Embodiments of systems and methods for preparing, using, and analyzing compositions comprising porous nitrocellulose strips are described herein. The following claims are directed to said embodiments, but do not preempt such compositions, methods and systems in the abstract. Those having skill in the art will recognize numerous other approaches to preparing and analyzing results on nitrocellulose strips are possible and/or utilized commercially, precluding any possibility of preemption in the abstract. However, the claimed system improves, in one or more specific ways, the operation of a machine system for producing nitrocellulose strips and analyzing assays made use such strips, and thus distinguishes from other approaches to the same problem/process in how its physical arrangement of a system determines the system's operation and ultimate effects on the material environment.

1. A device, comprising:
a housing comprising an upper first portion and a housing base, wherein the upper first portion comprises a sample port;
a nitrocellulose strip disposed on a planar surface of a substrate, wherein the substrate is enclosed by the housing between the upper first portion and the housing base; and
at least one sealed liquid reagent pack coupled to the upper first portion and accessible via the housing base;
wherein the nitrocellulose strip comprises at least one analyte capture region, the analyte capture region comprising one or more binding ligands.

2. The device of claim 1, wherein the nitrocellulose strip is one or more of linear, curved, S-shaped, sinuous, and angled.

3. The device of claim 1, wherein the substrate further comprises a computer readable identifier.

4. The device of claim 1, wherein the upper first portion of the housing comprises at least one window, wherein the at least one window is positioned over at least one of the at least one analyte capture regions.

5. The device of claim 1, wherein the nitrocellulose strip further comprises one or more partitions, the partitions positioned between at least two analyte capture locations, the partitions not including binding ligands.

6. The device of claim 1, further comprising a cover coupled to the upper first portion, the cover removably enclosing the sample port in the upper first portion of the housing.

7. The device of claim 1, further comprising lyophilized probe molecules on a fluidic gasket, wherein the fluidic gasket is enclosed by the housing and located between the upper first portion and the substrate.

8. The device of claim 1, wherein the upper first portion and the housing base are configured to be separated to remove or replace the substrate with the nitrocellulose strip.

9. The device of claim 1, wherein the binding ligands are for biomarkers for a plurality of diseases, wherein the diseases have different origins and a same symptomology.

10. A system for an optical assembly, comprising:
a first light source configured to illuminate fluorescently labeled ligands bound to a microarray on a substrate with a first wavelength of light;
a second light source configured to illuminate the fluorescently labeled ligands bound to the microarray on the substrate with a second wavelength of light, wherein the first and second light sources are parallel to one another and wherein the light from the first light source and light from the second light source are angled to engage with the fluorescently labeled ligands bound to the microarray on the substrate;
a camera configured to capture light emitted by the fluorescently labeled ligands, the camera positioned parallel to and between the first light source and the second light source, wherein the camera is centered on the fluorescently labeled ligands bound to the microarray on the substrate;
a plurality of switchable detection filters positioned between the camera and the fluorescently labeled ligands bound to the microarray on the substrate along a common axis,
wherein the plurality of switchable detection filters includes a first filter for capturing light emitted by the fluorescently labeled ligands bound to the microarray on the substrate following illumination by the first light source and a second filter for capturing light emitted by the fluorescently labeled ligands bound to the microarray on the substrate following illumination by the second light source; and
wherein both of the first light source and the second light source are positioned off of the common axis of the camera and the plurality of switchable detection filters.

11. The system of claim 10, wherein the plurality of switchable detection filters are linearly moveable perpendicular to the common axis.

12. The system of claim 10, wherein the camera is one of a charge-coupled device and a complementary metal-oxide semiconductor camera.

13. The system of claim 10, further comprising an antireflective window positioned behind the plurality of switchable detection filters and in front of the substrate.

14. The system of claim 10, further comprising an identification reader.

15. A microarray assembly for simultaneously quantifying a plurality of biomarkers on a porous nitrocellulose membrane comprising:
a user interface;
a housing comprising a plurality of compartments;
an optical assembly disposed in a first compartment of the plurality of compartments of the housing, wherein the optical assembly comprises:
at least one light source emitting light of a defined wavelength;
a camera parallel to the at least one light source and positioned perpendicular to a sample insertion pocket;
a plurality of switchable detection filters positioned between the camera and the sample insertion pocket along a central axis, wherein the plurality of switchable detection filters includes a first filter for capturing a first wavelength of light and a second filter for capturing a second wavelength of light;
a controller disposed in a second compartment of the plurality of compartments of the housing, wherein the controller comprises computer readable instructions that when executed cause the controller to instruct the camera to capture a light emitted from the sample, wherein the controller is activated through the user interface; and
a power source.

16. The microarray assembly of claim 15, wherein the housing further comprises a series of ports for connecting peripheral devices.

17. The microarray assembly of claim 15, wherein the user interface has computer readable instructions that when executed manipulate a defined wavelength and frequency of the light source.

18. The microarray assembly of claim 15, wherein the position of the switchable detection filters is mechanically manipulateable.

19. The microarray assembly of claim 15, wherein the user interface is a touch screen.

20. The microarray assembly of claim 15, wherein the sample insertion pocket is configured to receive a substrate with a porous nitrocellulose strip printed with a microarray of binding ligands, wherein the porous nitrocellulose strip has been contacted with a biological sample and labeled with molecular probes.
